(19) United States
(12) Patent Application Publication
(10) Pub. No.: US 2025/0353856 A1
(43) Pub. Date: Nov. 20, 2025

(54) COMPOUND ACTIVE AS INHIBITOR OF COLONY STIMULATION FACTOR-1 RECEPTOR (CSF-1R)

(71) Applicant: Norwegian University of Science and Technology (NTNU), Trondheim (NO)

(72) Inventors: Thomas Ihle AARHUS, Trondheim (NO); Eirik SUNDBY, Trondheim (NO); Bård Helge HOFF, Trondheim (NO); Frithjof BJØRNSTAD, Trondheim (NO); Geir BJØRKØY, Trondheim (NO)

(21) Appl. No.: 19/120,416

(22) PCT Filed: Oct. 13, 2023
(86) PCT No.: PCT/EP2023/078550

§ 371 Date: Apr. 11, 2025

(30) Foreign Application Priority Data
Oct. 13, 2022 (GB) 2215132.8

(51) Int. Cl. C07D 487/04; A61K 31/519; A61K 31/5377; A61K 31/675; C07F 9/6561

(52) U.S. Cl. CPC C07D 487/04; A61K 31/519; A61K 31/5377; A61K 31/675; C07F 9/6561

(57) ABSTRACT

The present invention relates to new pyrrolopyrimidines and purines which surprisingly act as CSF-1R inhibitors, to processes for making these compounds, and to uses thereof.

### SUMMARY OF THE INVENTION

The present invention relates to certain compounds, in particular new pyrrolopyrimidines and purines. These new compounds have been found to be inhibitors of CSF-1R (Colony stimulation factor-1 receptor), and as a result offer potential in the treatment of a number of diseases, such as cancers, bone disorders, neurological diseases, inflammatory disorders and eye diseases. The invention also relates to pharmaceutical compositions comprising said compounds, to the compounds for use in the treatment of various diseases, to methods of treating diseases by administration of said compounds, to the use of the compounds in the manufacture of a medicament for the treatment or prevention of various diseases, and to processes for the formation of said compounds.

### BACKGROUND TO THE INVENTION

CSF-1R (Colony stimulating factor-1 receptor) has emerged as an important pharmaceutical target as it plays an important role in a number of diseases (J. Med. Chem. 2018, 61, 13, 5450-5466, Journal for Immuno Therapy of Cancer (2017) 5:53, Biomedicine & Pharmacotherapy 103 (2018) 662-679). Colony stimulating factor-1 (CSF-1) is a common proinflammatory cytokine responsible for various disorders, which acts by binding to the receptor CSF-1R. CSF-1R or its activator the cytokine colony stimulation factor-1 (CSF1) is over expressed in many disorders and consequently is an important target. A number of inhibitors have been investigated, such as ABT-869, Imatinib, AG013736, JNJ-40346527, PLX3397, DCC-3014 and Ki20227. A few candidates have started clinical trials, but many side effects have been noted. The successful targeting of CSF-1R is therefore of importance to the pharmaceutical industry, and given its role in a number of diseases, even offers the possibility of multifunctional therapy (Biomedicine & Pharmacotherapy 103 (2018) 662-679).

Upon binding of CSF1 or IL-34 (interleukin 34) to the extracellular domain of CSF1R, also called M-CSFR (Macrophage colony-stimulating factor receptor) or CD115 (Cluster of Differentiation 115), the cell-surface receptor undergoes oligomerization and subsequent phosphorylation of tyrosine residues in the cytoplasmic kinase domain activating signal transduction. The kinase receptor mediates signalling responsible for the survival, function, proliferation and differentiation of cells in the myeloid lineage such as monocytes, macrophages, microglia and osteoclasts. (see Chitu, V.; Caescu, C. I.; R., S.; Lennartsson, J.; Rönnstrand, L.; Heldin, C. H., The PDGFR Receptor family, In: Receptor Tyrosine Kinases: Family and Subfamilies. Springer International Publishing, Switzerland: 2015; p 375-525)

The present group previously reported in WO2015/000959 certain new pyrrolo-, thieno-, and furo-[2,3-d]pyrimidine compounds and in particular, a series of such new 4-amino-6-aryl [2,3-d]pyrimidines. These compounds were found to be epidermal growth factor receptor tyrosine kinase (EGFR-TK) inhibitors and therefore offered potential in the treatment of cancer. The reported compounds all had a two-atom linker (NH—CRH) at the C-4 position of the bicyclic ring. The present inventors have surprisingly found that compounds with a single atom linker in the C-4 position are potent CSF-1R-inhibitors. This, unexpectedly, opens up a whole new field of therapeutic targeting for a number of diseases.

The present inventors have thus devised a class of compounds which surprisingly act as CSF-1R inhibitors, with structures typically based on pyrrolopyrimidines and purines. Given the known importance of targeting CSF-1R for a wide range of conditions, the present compounds represent a valuable new pharmaceutical class.

### SUMMARY OF THE INVENTION

In a first aspect, the invention provides a compound of formula (I)

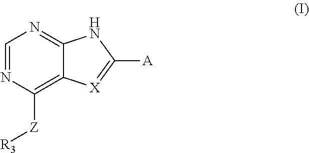

wherein:
   X is N or CH, preferably CH;
   Z is NR², O, S, or CHR², wherein R² is hydrogen, halogen (preferably F), methyl or ethyl, or deuterated or partially deuterated methyl or ethyl;
   A is a 5- or 6-membered hydrocarbyl or heterocyclic ring which may be optionally substituted with at least one R¹ group;
   each R¹ is independently selected from halogen, hydroxyl, —OCF₃, —CF₃, —CF₂H, —OCF₂H, CH(CF₃)OH, —C₁₋₆-alkyl, —O—C₁₋₆-alkyl, —O—(C₁-C₆ alkyl)-CH₂F, —O—(C₁-C₆ alkyl)-CHF₂, —O—(C₁-C₆ alkyl)-CF₃, —C₁₋₆alkyl-OH, —[C₁₋₆alkyl]ₘ-COOH, —[C₁₋₆alkyl]ₘ-COOC₁₋₆alkyl, —[C₁₋₆alkyl]ₘ-OCOC₁₋₆alkyl, —OCOR, —CO—[C₁₋₆alkyl]-COOH, —CO—[C₁₋₆alkyl]-OOOR, C(CH₃)₂—CH₂OH, —C(OH)₂(C₁₋₆alkyl-OH), —C(OH)(C₁₋₆alkyl-OH)₂, —C(C₁₋₆alkyl-OH)₃, C(CH₃)₂OH, —NH₂, NHR, —NR₂, —NO₂, —SO₂NH₂, —NHSO₂R, —NRSO₂R, —SO₂NHR, —SO₂NR₂, —POR₂, pyridyl (preferably ortho-pyridyl), cyclopropyl or cyclobutyl substituted with an —OH group, C₁-C₁₀ aliphatic hydrocarbyl group comprising at least one heteroatom selected from O, or N, a —O—C₁-C₁₀ aliphatic hydrocarbyl group containing at least one heteroatom selected from O or N, or a —[CH₂]ₘ-heterocycle wherein said heterocycle is optionally substituted with ═O or —OH;
   each m is 0 or 1;
   R³ is a 5- or 6-membered hydrocarbyl or heterocyclic ring either of which may be optionally substituted with one or more groups independently selected from C₁-C₄ alkyl, hydroxyl, halogen, —CF₃, —CF₂H, —NR₂, —NHCOR, —CO₂H, —CO₂R, or —OR;
   each R is C₁-C₆ alkyl;
or a pharmaceutically acceptable salt or solvate thereof;
   with the provisos that
   when Z is O or NH, then A is not phenyl substituted with a meta-NH₂ group;
      and when Z is NH and A is phenyl, then R³ is not phenyl substituted with both —OH and —OMe.

In a further aspect of the invention, there is provided a pharmaceutical composition comprising a compound as defined herein and at least one excipient.

In a further aspect of the invention, there is provided a compound defined herein for use in the treatment or prevention of a bone disorder, neurological disease, inflammatory disorder, cancer, or eye disease.

In a further aspect of the invention, there is provided a method of treating or preventing a bone disorder, neurological disease, inflammatory disorder, cancer, or eye disease, comprising administering a compound as defined herein to a subject in need thereof, optionally in combination with other chemotherapy agents or radiotherapy when administered for treating or preventing cancer.

In a further aspect of the invention, there is provided the use of a compound as defined herein in the manufacture of a medicament for the treatment or prevention of a bone disorder, neurological disease, inflammatory disorder, cancer or eye disease.

In a further aspect of the invention, there is provided a process for the preparation of a compound as defined herein, wherein said process comprises the steps of:
reacting a compound of formula:

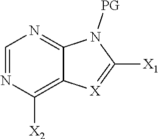

with
i) a compound of formula (RO)₂B-A or [A-BF₃]⁻ in the presence of a transition metal catalyst, wherein any OH groups in the R¹ substituent(s) of group A are optionally silane protected (e.g. with TBDMS), and
ii) a compound of formula —ZnBr—(CHR²)—R³ or —ZnBr—(CH₂)—R³ in the presence of a transition metal catalyst, or a compound of formula —OH—R³, —SH—R³, —(NHR²)—R³, or —(NH₂)—R³;
   in either order (i.e. i) then ii) or ii) then i));
then
iii) removing the protecting group PG;
   wherein
X₁ and X₂ are halogen;
PG is a protecting group, preferably selected from —SEM, THP, BOC, Cbz, Fmoc, SO₂Ph, Ts, MOM, CO₂H;
R is OH, OMe, or (RO)₂ is pinacol (i.e. —O—C(CH₃)₂—C(CH₃)₂—O—) or MIDA ester (i.e. —O—CO—CH₂—N(CH₃)—CH₂—CO—O—),
A, X, and R¹-R³ are as defined herein.

### Definitions

The following definitions may apply to all claimed compounds, unless otherwise indicated.

Any C₁₋₆ alkyl group is preferably a linear C₁₋₆ alkyl group. It is preferably a C₁₋₃ alkyl group, such as a linear C₁₋₃alkyl group, especially methyl, ethyl or n-propyl. However, any C₁₋₆ alkyl group can also be branched, e.g. —C(CH₃)₂—CH₂—, —C(CH₃)₂— etc.

When there are two or more ‘R’ groups or two or more ‘C₁₋₆-alkyl’ groups on a compound (e.g. —NRSO₂R or —[C₁₋₆alkyl]ₘ-OCOC₁₋₆alkyl), then each R group or each C₁₋₆ group is independently selected from C₁-C₆ alkyl, i.e. the two or more R groups or the two or more C₁₋₆ alkyl groups may be different.

Any halogen or Hal, which stand for halogen/halide, is preferably Cl or F, ideally F. X₁ and X₂ are typically Cl and/or I.

The term hydrocarbyl ring is meant to include both aliphatic and aromatic hydrocarbyl rings. Aliphatic herein means non-aromatic, and can include alkyl, but also alkenyl, alkynyl etc. Aliphatic can include cyclic, acyclic, or a mixture of both.

Heterocyclic herein means both non-aromatic heterocycles and also heteroaryl groups. The heterocyclic groups may include one, two or three heteroatoms, preferably one or two, most preferably one. Preferably, heterocyclic means herein a heterocycle with one heteroatom, typically selected from N, O and S.

By ‘optionally substituted’ is meant unsubstituted or substituted.

### DETAILED DESCRIPTION OF THE INVENTION

The invention relates to new compounds as CSF-1R inhibitors. The compounds preferably contain a substituted unit of formula (I)

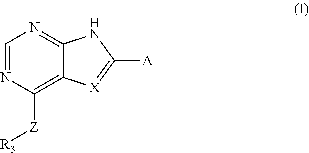

wherein:
   X is N or CH, preferably CH;
   Z is NR², O, S, or CHR², wherein R² is hydrogen, halogen (preferably F), methyl or ethyl, or deuterated or partially deuterated methyl or ethyl;
   A is a 5- or 6-membered hydrocarbyl or heterocyclic ring which may be optionally substituted with at least one R¹ group;
   each R¹ is independently selected from halogen, hydroxyl, —OCF₃, —CF₃, —CF₂H, —OCF₂H, CH(CF₃)OH, —C₁₋₆-alkyl, —O—C₁₋₆-alkyl, —O—(C₁-C₆ alkyl)-CH₂F, —O—(C₁-C₆ alkyl)-CHF₂, —O—(C₁-C₆ alkyl)-CF₃, —C₁₋₆alkyl-OH, —[C₁₋₆alkyl]ₘ-COOH, —[C₁₋₆alkyl]ₘ-COOC₁₋₆alkyl, —[C₁₋₆alkyl]ₘ-OCOC₁₋₆alkyl, —OCOR, —CO—[C₁₋₆alkyl]-COOH, —CO—[C₁₋₆alkyl]-COOR, C(CH₃)₂—CH₂OH, —C(OH)₂(C₁₋₆alkyl-OH), —C(OH)(C₁₋₆alkyl-OH)₂, —C(C₁₋₆alkyl-OH)₃, C(CH₃)₂OH, —NH₂, NHR, —NR₂, —NO₂, —SO₂NH₂, —NHSO₂R, —NRSO₂R, —SO₂NHR, —POR₂, pyridyl (preferably ortho-pyridyl), cyclopropyl or cyclobutyl substituted with an —OH group, C₁-C₁₀ aliphatic hydrocarbyl group comprising at least one heteroatom selected from O, or N, a —O—C₁-C₁₀ aliphatic hydrocarbyl group containing at least one heteroatom selected from O or N, or a —[CH₂]ₘ-heterocycle wherein said heterocycle is optionally substituted with ═O or —OH;
   each m is 0 or 1;
   R³ is a 5- or 6-membered hydrocarbyl or heterocyclic ring either of which may be optionally substituted with one or more groups independently selected from C₁-C₄ alkyl, hydroxyl, halogen, —CF₃, —CF₂H, —NR₂, —NHCOR, —CO₂H, —CO₂R, or —OR;
   each R is C₁-C₆ alkyl;
or a pharmaceutically acceptable salt or solvate thereof;
   with the provisos that
   when Z is O or NH, then A is not phenyl substituted with a meta-NH₂ group;
      and when Z is NH and A is phenyl, then R³ is not phenyl substituted with both —OH and —OMe.

The discussion of the various groups below are applicable to all embodiments and aspects of the invention, where technically viable, and are all combinable with one another.

X is N or CH, preferably CH.

In a particular embodiment, therefore the compound is selected from pyrrolopyrimidines and purines.

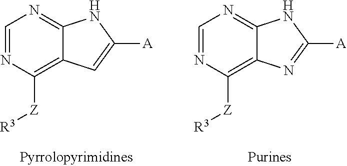

Pyrrolopyrimidines are preferred.

Z is NR², O, S, or CHR², wherein R² is hydrogen, halogen (preferably F), methyl or ethyl, or deuterated or partially deuterated methyl or ethyl. Preferably Z is NH, O, S, or CHR². Preferably Z is O, S, or CHR². Preferably Z is O or CHR², more preferably CHR². R² is preferably H, F or Me. When Z=NR², then R² is not halogen.

In all embodiments of the invention, A is a 5- or 6-membered hydrocarbyl or heterocyclic ring optionally substituted with at least one group R¹ group.

The 5-membered ring may be substituted with 0-4 R¹ groups, preferably 0-3 R¹ groups, more preferably 0-2 R¹ groups, more preferably 0-1 R¹ groups, more preferably 1 R¹ group. Each R¹ group may be the same or different. The 6-membered ring may be substituted with 0-5 R¹ groups, preferably 0-4 R¹ groups, more preferably 0-3 R¹ groups, more preferably 0-2 R¹ groups, more preferably 1-2 R¹ groups or 0-1 R¹ groups, more preferably 1 R¹ group, more preferably 1 R¹ group in para-position. Each R¹ group may be the same or different. The presence of a ring at the A position can increase CSF-1R potency.

In a particular embodiment, A is a 5- or 6-membered hydrocarbyl or heterocyclic ring substituted with at least one group R¹ group.

In a particular embodiment, A is 5- or 6-membered heterocyclic ring or a 5-membered hydrocarbyl ring. In a particular embodiment, A is a 5-membered hydrocarbyl or heterocyclic ring or 6-membered heterocyclic ring, each of which may be optionally substituted with at least one R¹ group or A is a 6-membered hydrocarbyl ring substituted with at least one R¹ group.

Each R¹ is independently selected from halogen, hydroxyl, —OCF₃, —CF₃, —CF₂H, —OCF₂H, CH(CF₃)OH, —C₁₋₆-alkyl, —O—C₁₋₆-alkyl, —O—(C₁-C₆ alkyl)-CH₂F, —O—(C₁-C₆ alkyl)-CHF₂, —O—(C₁-C₆ alkyl)-CF₃, —C₁₋₆alkyl-OH, —[C₁₋₆alkyl]ₘ-COOH, —[C₁₋₆alkyl]ₘ-COOC₁₋₆alkyl, —[C₁₋₆alkyl]ₘ-OCOC₁₋₆alkyl, —OCOR, —CO—[C₁₋₆alkyl]-COOH, —CO—[C₁₋₆alkyl]-COOR, C(CH₃)₂—CH₂OH, —C(OH)₂(C₁₋₆alkyl-OH), —C(OH)(C₁₋₆alkyl-OH)₂, —C(C₁₋₆alkyl-OH)₃, C(CH₃)₂OH, —NH₂, NHR, —NR₂, —NO₂, —SO₂NH₂, —NHSO₂R, —NRSO₂R, —SO₂NHR, —SO₂NR₂, —POR₂, pyridyl (preferably ortho-pyridyl), cyclopropyl or cyclobutyl substituted with an —OH group, C₁-C₁₀ aliphatic hydrocarbyl group comprising at least one heteroatom selected from O, or N, a —O—C₁-C₁₀ aliphatic hydrocarbyl group containing at least one heteroatom selected from O or N, or a —[CH₂]ₘ-heterocycle wherein said heterocycle is optionally substituted with ═O or —OH; wherein R is C₁-C₆ alkyl and each m is 0 or 1.

In a particular embodiment, R¹ is not —NH₂.

In a particular embodiment, R¹ is not halogen.

In a particular embodiment, R¹ is not halogen or —NH₂.

Preferably each R¹ is independently selected from halogen, hydroxyl, —OCF₃, —CF₃, —CF₂H, —OCF₂H, CH(CF₃)OH, —O—C₁₋₆-alkyl, —C₁₋₆alkyl-OH, —[C₁₋₆alkyl]ₘ-COOH, —[C₁₋₆alkyl]ₘ-COOC₁₋₆alkyl, —[C₁₋₆alkyl]ₘ-OCOC₁₋₆alkyl, —OCOR, —CO—[C₁₋₆alkyl]-COOH, —CO—[C₁₋₆alkyl]-COOR, C(CH₃)₂—CH₂OH, C(CH₃)₂OH, NHR, —NR₂, —NO₂, —SO₂NH₂, —NHSO₂R, —SO₂NHR, —POR₂, pyridyl (preferably ortho-pyridyl), cyclopropyl or cyclobutyl substituted with an —OH group, C₁-C₁₀ aliphatic hydrocarbyl group comprising at least one heteroatom selected from O, or N, a —O—C₁-C₁₀ aliphatic hydrocarbyl group containing at least one heteroatom selected from O or N, or a —[CH₂]ₘ-heterocycle wherein said heterocycle is optionally substituted with ═O; wherein R is C₁-C₆ alkyl and each m is 0 or 1.

Preferably, each R¹ is independently selected from hydroxyl, —OCF₃, —CF₃, —CF₂H, —OCF₂H, CH(CF₃)OH, —C₁₋₆-alkyl, —O—C₁₋₆-alkyl, —O—(C₁-C₆ alkyl)-CH₂F, —O—(C₁-C₆ alkyl)-CHF₂, —O—(C₁-C₆ alkyl)-CF₃, —C₁₋₆alkyl-OH, —[C₁₋₆alkyl]ₘ-COOH, —[C₁₋₆alkyl]ₘ-COOC₁₋₆alkyl, —[C₁₋₆alkyl]ₘ-OCOC₁₋₆alkyl, —OCOR, —CO—[C₁₋₆alkyl]-COOH, —CO—[C₁₋₆alkyl]-COOR, C(CH₃)₂—CH₂OH, —C(OH)₂(C₁₋₆alkyl-OH), —C(OH)(C₁₋₆alkyl-OH)₂, —C(C₁₋₆alkyl-OH)₃, C(CH₃)₂OH, —NH₂, NHR, —NR₂, —NO₂, —SO₂NH₂, —NHSO₂R, —NRSO₂R, —SO₂NHR, —SO₂NR₂, —P(O)R₂, pyridyl (preferably ortho-pyridyl), cyclopropyl or cyclobutyl substituted with an —OH group, C₁-C₁₀ aliphatic hydrocarbyl group comprising at least one heteroatom selected from O, or N, a —O—C₁-C₁₀ aliphatic hydrocarbyl group containing at least one heteroatom selected from O or N, or a —[CH₂]ₘ-heterocycle wherein said heterocycle is optionally substituted with ═O or —OH

Preferably, each R¹ is independently selected from hydroxyl, —OCF₃, —CF₃, —CF₂H, —OCF₂H, CH(CF₃)OH, —C₁₋₆-alkyl, —O—C₁₋₆-alkyl, —O—(C₁-C₆ alkyl)-CH₂F, —O—(C₁-C₆ alkyl)-CHF₂, —O—(C₁-C₆ alkyl)-CF₃, —C₁₋₆alkyl-OH, —[C₁₋₆alkyl]ₘ-COOH, —[C₁₋₆alkyl]ₘ-COOC₁₋₆alkyl, —[C₁₋₆alkyl]ₘ-OCOC₁₋₆alkyl, —OCOR, —CO—[C₁₋₆alkyl]-COOH, —CO—[C₁₋₆alkyl]-COOR, C(CH₃)₂—CH₂OH, —C(OH)₂(C₁₋₆alkyl-OH), —C(OH)(C₁₋₆alkyl-OH)₂, —C(C₁₋₆alkyl-OH)₃, C(CH₃)₂OH, NHR, —NR₂, —NO₂, —SO₂NH₂, —NHSO₂R, —NRSO₂R, —SO₂NHR, —SO₂NR₂, —P(O)R₂, pyridyl (preferably ortho-pyridyl), cyclopropyl or cyclobutyl substituted with an —OH group, C₁-C₁₀ aliphatic hydrocarbyl group comprising at least one heteroatom selected from O, or N, a —O—C₁-C₁₀ aliphatic hydrocarbyl group containing at least one heteroatom selected from O or N, or a —[CH₂]ₘ-heterocycle wherein said heterocycle is optionally substituted with ═O or —OH.

Preferably, each R¹ is independently selected from Cl, F, —OCF₃, —CF₃, —CF₂H, —OCF₂H, CH(CF₃)OH, —C₁₋₆-alkyl, —O—C₂₋₆-alkyl, —O—(C₁-C₆ alkyl)-CH₂F, —O—(C₁-C₆ alkyl)-CHF₂, —O—(C₁-C₆ alkyl)-CF₃, —C₁₋₆alkyl-OH, —[C₁₋₆alkyl]ₘ-COOH, —[C₁₋₆alkyl]ₘ-COOC₁₋₆alkyl, —[C₁₋₆alkyl]ₘ-OCOC₁₋₆alkyl, —OCOR, —CO—[C₁₋₆alkyl]-COOH, —CO—[C₁₋₆alkyl]-COOR, C(CH₃)₂—CH₂OH, —C(OH)₂(C₁₋₆alkyl-OH), —C(OH)(C₁₋₆alkyl-OH)₂, —C(C₁₋₆alkyl-OH)₃, C(CH₃)₂OH, —NH₂, NHR, —NR₂, —NO₂, —SO₂NH₂, —NHSO₂R, —NRSO₂R, —SO₂NHR, —SO₂NR₂, —P(O)R₂, pyridyl (preferably ortho-pyridyl), cyclopropyl or cyclobutyl substituted with an —OH group, C₁-C₁₀ aliphatic hydrocarbyl group comprising at least one heteroatom selected from O, or N, a —O—C₁-C₁₀ aliphatic hydrocarbyl group containing at least one heteroatom selected from O or N, or a —[CH₂]ₘ-heterocycle wherein said heterocycle is optionally substituted with ═O or —OH.

Preferably, R¹ is selected from —C₁₋₆alkyl-OH, —O—C₁₋₆-alkyl, —C₁₋₆-alkyl, —NHSO₂R, —SO₂NHR, —[CH₂]ₘ-heterocycle wherein said heterocycle is optionally substituted with ═O or —OH.

Preferably, R¹ is —C₁₋₆alkyl-OH or —O—C₁₋₆-alkyl, preferably —C₁₋₃alkyl-OH or —O—C₁₋₃-alkyl, more preferably —C₁₋₂alkyl-OH or —O—C₁₋₂-alkyl, more preferably —(CH₂)—OH or —OCH₃, more preferably para —(CH₂)—OH or para —OCH₃.

It has been found that polar R¹ groups typically result in increased potency in terms of CSF-1R inhibition. This is likely caused by favourable polar interactions at the surface of the binding pocket. The R¹ group typically protrudes out of the ligand-protein complex when the structure is bound to CSF-1R. The R¹ group(s) can thus be relatively large, and can potentially be used to increase the solubility and tune other pharmacokinetic properties of the compound, without affecting its ability to bind to the receptor. Lack of water solubility is typically a problem for pharmaceuticals and whilst our compounds are not typically water soluble in non-salt form, the increased polarity for certain R¹ groups can increase bioavailability.

When R¹ is —C₁₋₆-alkyl, R¹ may typically be ⁱPr or Me. Typically, R¹ is —C₁₋₆-alkyl when A is pyrazolyl.

When R¹ is a C₁-C₁₀ aliphatic hydrocarbyl group comprising at least one heteroatom selected from O or N, the O or N atoms are typically within the C₁-C₁₀ group, i.e. embedded within the chain, or at a terminal position, i.e. the end of the chain. Typically, said C₁-C₁₀ aliphatic hydrocarbyl group containing at least one heteroatom selected from O and N is a C₁-C₁₀, preferably C₁-C₈, preferably C₁-C₆ aliphatic hydrocarbyl group containing at least one group selected from ether, carboxylic acid, ester, alcohol, primary amine, secondary amine, tertiary amine, amide, preferably selected from ether, alcohol, primary amine, secondary amine, tertiary amine. The R¹ group in this case may contain a cyclic group or may be acyclic. The aliphatic hydrocarbyl group is typically, however, non-cyclic in this instance. Suitable groups therefore include C₁-C₁₀ aliphatic hydrocarbyl groups comprising at least one ether group, at least one —NH— group, at least one —NR— group, at least one —NHR group, or at least one —NR₂ group, wherein R is a C₁-C₆ alkyl group. A suitable R¹ group in this case could be, for example:

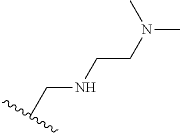 [SMILES: *CNCCN(C)C]

When R¹ is a —O—C₁-C₁₀ aliphatic hydrocarbyl group containing at least one heteroatom selected from O or N, the same considerations apply for the substitutions with the O or N groups. The group may be acyclic or may be cyclic or comprise a cyclic moiety. If it is cyclic or comprises a cyclic moiety, the R¹ group preferably is or comprises a O- or N-heterocycle, preferably an O-heterocycle. Suitable R¹ groups in this case include, for example, —O—[CH₂—CH₂—O]_{z}—CH₃ (i.e. ‘PEG’-type groups), with z being 1-6, preferably 1-4, more preferably 1-3, e.g. 1 or 3. Suitable R¹ groups in this case could be:

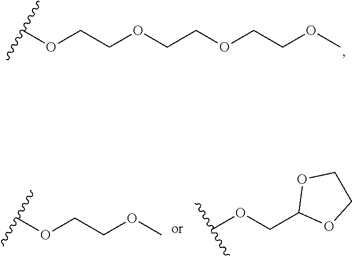

When R¹ is a —[CH₂]ₘ-heterocycle wherein said heterocycle is optionally substituted with ═O′, the heterocycle may be any non-aromatic or aromatic heterocycle (typically 5- or 6-membered), e.g. containing one or two atoms selected from N, O or S (preferably 0, or both N and O). The ═O substitution, if present, is typically on the ring (e.g. as in lactones). Particular examples of such R¹ groups include:

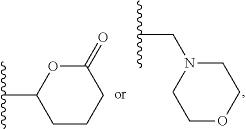

in particular

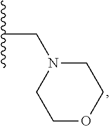 [SMILES: *CN1CCOCC1]

For R¹, a particularly preferred cyclopropyl or cyclobutyl substituted with an —OH group is:

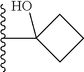 [SMILES: *C1(O)CCC1]

As mentioned above the A group in all embodiments of the invention is a 5- or 6-membered hydrocarbyl or heterocyclic ring optionally substituted with at least one group R¹ group. Said hydrocarbyl ring may be selected from phenyl, cyclopentyl, cyclohexenyl, cyclopentenyl, cyclopentadienyl, cyclohexadienyl, and cyclohexyl, preferably phenyl. Said heterocyclic ring is typically either a non-aromatic heterocyclic ring comprising at least one (e.g. 1 or 2) heteroatom selected from N, O, or S, e.g. piperidine, tetrahydropyran, tetrahydrofuran, dihydropyran, morpholine, tetrahydrofuran, pyrrolidine, or a heteroaryl ring comprising at least one (e.g. 1 or 2) heteroatom selected from N, O, or S, e.g. pyridine, thiophene, furan, imidazole, pyrazole, thiophene, furan, pyrrole, thiazole, thiophene, oxazole, isoxazole. In a particular embodiment, the 6-membered hydrocarbyl or heterocyclic ring in the A group is selected from phenyl, cyclohexenyl, pyridyl, morpholinyl, dihydropyranyl. In a particular embodiment, the 6-membered hydrocarbyl or heterocyclic ring in the A group is selected from phenyl, cyclohexenyl, morpholinyl, dihydropyranyl. In a particular embodiment, the 5-membered hydrocarbyl or heterocyclic ring in the A group is selected from imidazole, pyrazole or thiophene, preferably imidazole or pyrazole. Preferable A groups are phenyl or pyrazolyl, each optionally substituted with at least one group R¹ group. Typically, the ring is a phenyl ring. Any of the above may obviously optionally be substituted with at least one R¹ group.

Most preferably, A is phenyl optionally substituted with one R¹ group selected from —C₁₋₆alkyl-OH, —O—C₁₋₆-alkyl, —C₁₋₆-alkyl, —NHSO₂R, —SO₂NHR, —[CH₂]ₘ-heterocycle wherein said heterocycle is optionally substituted with ═O or —OH.

When Z=O, A is typically substituted with at least one R¹ group, preferably phenyl substituted with at least one R¹ group.

When Z is NH, and R³ is a heterocyclic ring, A is preferably substituted with at least one R¹ group.

Most preferably, A is phenyl optionally substituted with one R¹ group selected from —C₁₋₆alkyl-OH or —O—C₁₋₆-alkyl, preferably —C₁₋₃alkyl-OH or —O—C₁₋₃-alkyl, more preferably —C₁₋₂alkyl-OH or —O—C₁₋₂-alkyl, more preferably —(CH₂)—OH or —OCH₃, more preferably para —(CH₂)—OH or para —OCH₃.

R³ is a 5- or 6-membered hydrocarbyl or heterocyclic ring either of which may be optionally substituted with one or more groups (preferably one group) independently selected from C₁-C₄ alkyl, hydroxyl, halogen, —CF₃, —CF₂H, —NR₂, —NHCOR, —CO₂H, —CO₂R, or —OR. Each R is C₁-C₆ alkyl. C₁-C₄ and C₁-C₆ alkyl herein covers both linear and branched C₁-C₄/C₆ alkyl.

For R³, the 5- or 6-membered hydrocarbyl ring is typically an aliphatic or aromatic hydrocarbyl ring preferably selected from phenyl, cyclopentyl, and cyclohexyl, preferably selected from phenyl and cyclopentyl, preferably phenyl. The 5- or 6-membered heterocyclic ring is typically either a non-aromatic heterocyclic ring comprising one heteroatom selected from N, O, or S, e.g. piperidine, tetrahydropyran, tetrahydrofuran, or a heteroaryl ring comprising one heteroatom selected from N, O, or S, e.g. pyridine, thiophene, furan. Alternatively, when R₃ is a 5- or 6-membered heterocyclic ring, it is a 5- or 6-membered heterocyclic ring comprising one or two heteroatoms, preferably one heteroatom.

Any of these rings can obviously be substituted, as per the optional substituents listed above. In a particular embodiment, R³ is a 5- or 6-membered hydrocarbyl or heterocyclic ring substituted with a methyl group, preferably a phenyl ring substituted with a methyl group, more preferably a phenyl ring substituted with a methyl group in the meta position.

Tetrahydropyran as R³ has a strong potency-increasing effect in addition to increasing the solubility of the compounds. In a particular embodiment, therefore, R³ is tetrahydropyranyl, in particular when Z is NR₂. 4-tetrahydropyranyl is preferred, i.e.

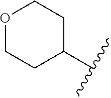 [SMILES: *C1CCOCC1]

In a particular embodiment, R³ is a 5- or 6-membered hydrocarbyl ring or 5- or 6-membered non-aromatic heterocyclic ring, any of which may be optionally substituted with one or more groups independently selected from C₁-C₄ alkyl, hydroxyl, halogen, —CF₃, —CF₂H, —NR₂, —NHCOR, —CO₂H, —CO₂R, or —OR.

In a particular embodiment, R³ is a 5- or 6-membered hydrocarbyl or heterocyclic ring either of which may be optionally substituted with one or more groups independently selected from C₁-C₄ alkyl, halogen, —CF₃, —CF₂H, —NR₂, —NHCOR, —CO₂H, or —CO₂R. In a particular embodiment, R³ is a 5-membered hydrocarbyl or heterocyclic ring either of which may be optionally substituted with one or more groups independently selected from C₁-C₄ alkyl, hydroxyl, halogen, —CF₃, —CF₂H, —NR₂, —NHCOR, —CO₂H, —CO₂R, or —OR; or R³ is a 6-membered hydrocarbyl or heterocyclic ring either of which is substituted by one or more groups independently selected from C₁-C₄ alkyl, halogen, —CF₃, —CF₂H, —NR₂, —NHCOR, —CO₂H, —CO₂R or —CO₂H.

In a particular embodiment, R³ is phenyl optionally substituted with one or more groups (preferably one group) independently selected from C₁-C₄ alkyl, hydroxyl, halogen, —CF₃, —CF₂H, —NR₂, —NHCOR, —CO₂H, —CO₂R, or —OR (wherein each R is C₁-C₆ alkyl), preferably wherein R³ is phenyl optionally substituted with C₁-C₄ alkyl, halogen (preferably F) or —CF₃. Most preferably, R³ is phenyl optionally substituted with F, Me, ⁱPr, ^{t}Bu, —CF3, preferably phenyl optionally substituted with F, para-Me, para-ⁱPr, para-^{t}Bu, para-CF₃. In a particular embodiment, R³ is substituted phenyl.

In a particular embodiment, R₃ is a 5- or 6-membered heterocyclic ring comprising one or two heteroatoms (only), preferably one heteroatom.

In a particular embodiment, R₃ is not oxadiazole. When Z=NR₂, R³ is typically a 5- or 6-membered heterocyclic ring, preferably a 6-membered non-aromatic heterocyclic ring, preferably a 6-membered saturated heterocyclic ring, such as tetrahydropyranyl.

When Z=NR₂, R³ is typically an O-based heterocyclic ring (i.e. a heterocyclic ring with only O as heteroatom).

When Z=O, R³ is typically unsubstituted phenyl.

The nature of the R³ group is important as it binds to an internal area of the CSF-1R protein. Because of the short one-atom ‘Z’ linker between the C-4 position of the bicyclic core and the R³ substituent, considerable flexibility is possible for the substitution at the R³ position, when compared to R³ groups that are tethered to the bicyclic core via a 2-atom linker.

In a particular embodiment, the compound of the invention is of formula (II):

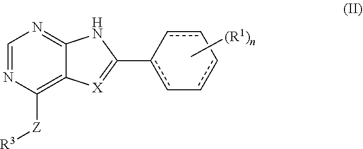

wherein:
   X, Z, R¹, R³, are as hereinbefore defined;
   each [symbol] independently represents a single or double bond, preferably
   each [symbol] represents a double bond;
   n is an integer between 0 and 3, preferably between 0 and 2, preferably 1 or 2, more preferably 1;
or a pharmaceutically acceptable salt or solvate thereof.

In a particular embodiment, the compounds of the invention (including compounds for use) are selected from the following compounds in Tables 1a and 1b. The invention provides for the grouping or exclusion of any of these compounds.

TABLE 1a
Structures of pyrrolopyrimidines.

| | |
|---|---|
| | 5a-k and 9 |
| 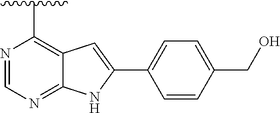 [SMILES: *c1ncnc2[nH]c(-c3ccc(CO)cc3)cc12] | |
| | 10 |
| 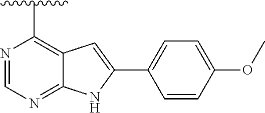 [SMILES: *c1ncnc2[nH]c(-c3ccc(OC)cc3)cc12] | |

| Compound | R |
|---|---|
| 5a (TIA03-170) | 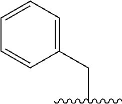 [SMILES: *Cc1ccccc1] |
| 5b (VT-44-03) | 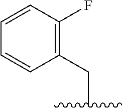 [SMILES: *Cc1ccccc1F] |
| 5c (VT-84-06) | 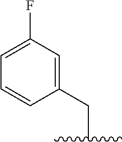 [SMILES: *Cc1cccc(F)c1] |
| 5d (VT-22-03) | 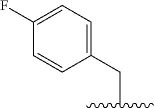 [SMILES: *Cc1ccc(F)cc1] |
| 5e (VT-64-03) | 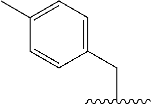 [SMILES: *Cc1ccc(C)cc1] |
| 5f(VT-82-03) | 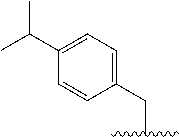 [SMILES: *Cc1ccc(C(C)C)cc1] |
| 5g (VT-28-02) | 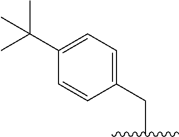 [SMILES: *Cc1ccc(C(C)(C)C)cc1] |
| 5h (VT-56-03) | 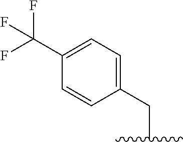 [SMILES: *Cc1ccc(C(F)(F)F)cc1] |
| 5i (VT-12-03; VT-10-05) | 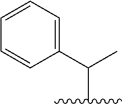 [SMILES: *C(C)c1ccccc1] |
| 5j (TIA05-134) | 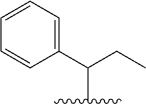 [SMILES: *C(CC)c1ccccc1] |
| 5k (TIA05-136) | 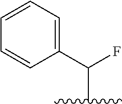 [SMILES: *C(F)c1ccccc1] |
| 9 (HIL120) | 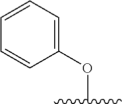 [SMILES: *Oc1ccccc1] |
| 10 (TIA03-164) | 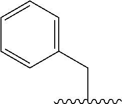 [SMILES: *Cc1ccccc1] |

TABLE 1b
Structures of additional pyrrolopyrimidines.

| Compound | Structure |
|---|---|
| SH-01-81 | 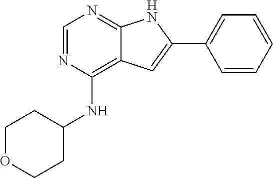 [SMILES: c1ccc(-c2cc3c(NC4CCOCC4)ncnc3[nH]2)cc1] |
| SH-01-98 | 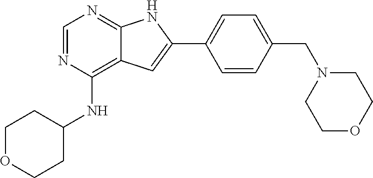 [SMILES: c1nc(NC2CCOCC2)c2cc(-c3ccc(CN4CCOCC4)cc3)[nH]c2n1] |
| LR-2-144 | 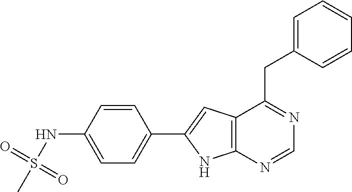 [SMILES: CS(=O)(=O)Nc1ccc(-c2cc3c(Cc4ccccc4)ncnc3[nH]2)cc1] |
| LR-2-163 | 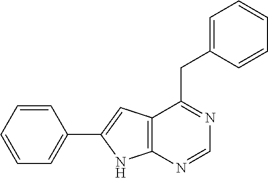 [SMILES: c1ccc(Cc2ncnc3[nH]c(-c4ccccc4)cc23)cc1] |
| LR-2-177 | 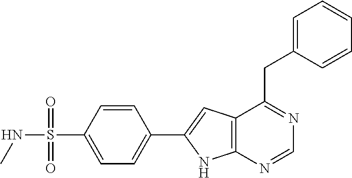 [SMILES: CNS(=O)(=O)c1ccc(-c2cc3c(Cc4ccccc4)ncnc3[nH]2)cc1] |
| LR-2-179 | 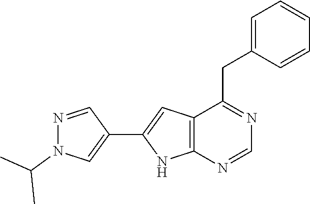 [SMILES: CC(C)n1cc(-c2cc3c(Cc4ccccc4)ncnc3[nH]2)cn1] |
| LR-2-185 | 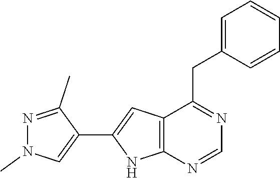 [SMILES: Cc1nn(C)cc1-c1cc2c(Cc3ccccc3)ncnc2[nH]1] |
| LR-3-002 | 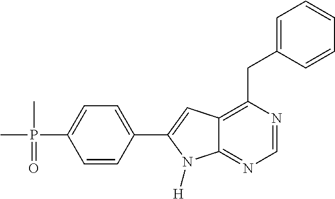 [SMILES: CP(C)(=O)c1ccc(-c2cc3c(Cc4ccccc4)ncnc3[nH]2)cc1] |

### Provisos

In a particular embodiment, when Z is O or NH, then A is not phenyl substituted with a meta-NH₂ group;
and when Z is NH and A is phenyl (i.e. unsubstituted phenyl), then R³ is not phenyl substituted with both —OH and —OMe.

Alternatively, when Z is NH, and A is phenyl substituted with a meta-NH₂ group, then R³ is typically not phenyl substituted with one of the following:
meta-OH and para-OMe
meta-OH and para-Me
3,4,5, tri-methoxy (i.e. —OMe in both meta-positions and the para-position)
pyridyl
meta-(NH)(COMe)
meta-COOH
meta-OH
para-OH
para-Me

Alternatively or additionally, when Z is O, and A is phenyl substituted with a meta-NH₂ group, then R³ is typically not meta-OH.

In a further alternative embodiment, if Z is O, then A is not unsubstituted phenyl or unsubstituted pyridyl, preferably wherein if Z is O, then A is not unsubstituted phenyl or unsubstituted pyridyl and R³ is not phenyl substituted with NH₂, NO₂ and/or F.

In a further embodiment, if X is N and Z is S, then R³ is not oxadiazole.

In a further embodiment, when X is N and R³ is a 5-membered heterocyclic ring, there are one or two heteroatoms in the 5-membered heterocyclic ring.

In a further embodiment, when X is N, Z is NH and A is unsubstituted phenyl, then R³ is not unsubstituted phenyl.

In a further embodiment, when X is N, Z is NH and R³ is unsubstituted phenyl, then A is not unsubstituted phenyl.

In a further embodiment, when X is N, R³ and A are not both unsubstituted phenyl.

In a further embodiment, when X is N and Z is O, R³ is not cyclopentyl.

In a further embodiment, when X is N, Z is O, A is not unsubstituted phenyl or unsubstituted pyridyl.

In a further embodiment, when X is N, Z is O, A is not unsubstituted phenyl or unsubstituted pyridyl.

In a further embodiment, when X is N and Z is O, R³ is not an unsubstituted 5-membered hydrocarbyl ring.

In a further embodiment, when X is N, Z is O or CHR², and in addition when Z is O, R³ is not an unsubstituted 5-membered hydrocarbyl ring.

In a further embodiment, when Z is S, then R³ is a 5- or 6-membered hydrocarbyl or heterocyclic ring either of which may be optionally substituted with one or more groups independently selected from C₁-C₄ alkyl, hydroxyl, halogen, —CF₃, —CF₂H, —NR₂, —CO₂H, —CO₂R, or —OR.

In a particular embodiment, when X is N and Z is NH, R³ is a 5- or 6-membered hydrocarbyl ring or non-aromatic heterocyclic ring, any of which may be optionally substituted with one or more groups independently selected from C₁-C₄ alkyl, hydroxyl, halogen, —CF₃, —CF₂H, —NR₂, —NHCOR, —CO₂H, —CO₂R, or —OR.

In a particular embodiment, when X is N and Z is NH, R¹ is not halogen.

In a particular embodiment, when X is N and Z is NH, R¹ is independently selected from hydroxyl, —OCF₃, —CF₃, —CF₂H, —OCF₂H, CH(CF₃)OH, —C₁₋₆-alkyl, —O—C₁₋₆-alkyl, —O—(C₁-C₆ alkyl)-CH₂F, —O—(C₁-C₆ alkyl)-CHF₂, —O—(C₁-C₆ alkyl)-CF₃, —C₁₋₆alkyl-OH, —[C₁₋₆alkyl]ₘ-COOH, —[C₁₋₆alkyl]ₘ-COOC₁₋₆alkyl, —[C₁₋₆alkyl]ₘ-OCOC₁₋₆alkyl, —OCOR, —CO—[C₁₋₆alkyl]-COOH, —CO—[C₁₋₆alkyl]-COOR, C(CH₃)₂—CH₂OH, —C(OH)₂(C₁₋₆alkyl-OH), —C(OH)(C₁₋₆alkyl-OH)₂, —C(C₁₋₆alkyl-OH)₃ C(CH₃)₂OH, —NH₂, NHR, —NR₂, —NO₂, —SO₂NH₂, —NHSO₂R, —NRSO₂R, —SO₂NHR, —SO₂NR₂, —POR₂, pyridyl (preferably ortho-pyridyl), cyclopropyl or cyclobutyl substituted with an —OH group, C₁-C₁₀ aliphatic hydrocarbyl group comprising at least one heteroatom selected from O, or N, a —O—C₁-C₁₀ aliphatic hydrocarbyl group containing at least one heteroatom selected from O or N, or a —[CH₂]ₘ-heterocycle wherein said heterocycle is optionally substituted with ═O or —OH.

In a particular embodiment, when X is N and Z is NH, R³ is not a 5- or 6-membered aromatic heterocyclic ring.

In a particular embodiment, when X is N and Z is NH, A is not unsubstituted pyridyl.

In a particular embodiment, when Z is NR², R³ is not cyclohexyl.

In a particular embodiment, when X is CH and Z is NCH₃, R³ is not unsubstituted cyclohexyl.

In a particular embodiment, when X is CH and Z is NCH₃, A is not unsubstituted phenyl.

In a particular embodiment, when X is CH and Z is NH, R¹ is not —OMe, hydroxyl or bromine.

In a particular embodiment when X is CH, Z is NR², O, S, or CHR², wherein R² is hydrogen, halogen (preferably F), methyl or ethyl, or deuterated or partially deuterated methyl or ethyl, then NR₂ is not NH.

In a particular embodiment, when X is CH and Z is O, R³ is not phenyl, optionally substituted with one or more groups selected from OH and —OMe.

In a particular embodiment, when X is CH and Z is O or S, R³ is a 5-membered hydrocarbyl or heterocyclic ring either of which may be optionally substituted with one or more groups independently selected from C₁-C₄ alkyl, hydroxyl, halogen, —CF₃, —CF₂H, —NR₂, —NHCOR, —CO₂H, —CO₂R, or —OR; or R³ is a 6-membered hydrocarbyl or heterocyclic ring either of which is substituted by one or more groups independently selected from C₁-C₄ alkyl, halogen, —CF₃, —CF₂H, —NR₂, —NHCOR, —CO₂H, —CO₂R or —CO₂H.

In a particular embodiment, when X is CH and Z is NCH₃, A is a 5-membered hydrocarbyl or heterocyclic ring or 6-membered heterocyclic ring, each of which may be optionally substituted with at least one R¹ group or A is a 6-membered hydrocarbyl ring substituted with at least one R¹ group.

In a particular embodiment, when X is CH, Z is O or CHR².

In a particular embodiment, when X is CH, Z is not NH and/or NMe.

In a particular embodiment, when X is CH, R¹ is not NH₂ and there is at least one R¹ group.

In a particular embodiment, when X is CH and Z is NCH₃, R³ is not cyclohexyl.

In a particular embodiment, when X is CH and Z is NCH₃, A is not unsubstituted phenyl.

In a particular embodiment, when A is phenyl there is at least one R¹ substituent.

In a particular embodiment, when X is CH and Z is NH, R³ is not phenyl optionally substituted with one or more groups independently selected from C₁-C₄ alkyl, hydroxyl, halogen, —CF₃, —CF₂H, —NR₂, —NHCOR, —CO₂H, —CO₂R or —OR; and R³ is not substituted pyrazole.

In a particular embodiment, when X is CH and Z is NH, R³ is not phenyl, pyrazole, pyridyl or pyridone optionally substituted with one or more groups independently selected from C₁-C₄ alkyl, hydroxyl, halogen, —CF₃, —CF₂H, —NR₂, —NHCOR, —CO₂H, —CO₂R, or —OR.

In a particular embodiment, when X is CH and Z is NH, R¹ is not —CH₂OH, —CH₂NH₂, —COOC₁₋₆alkyl, —CH₂N—(C₁₋₆alkyl)₂, —CH₂NH—(C₁-C₁₀ aliphatic hydrocarbon containing at least one N and/or O), —CH₂NHCO-pyridyl, —CON—(C₁₋₆alkyl)₂, —CO-(heterocycle containing at least one N), —CH₂-(heterocycle containing at least one N and/or O), wherein said heterocycle is optionally substituted with one or more R groups.

Any number of the above embodiments can be combined with one another, where technically viable.

In a particular embodiment, the compound is not one of the following (the following compounds from Table 2 can be excluded individually, or more than one compound can be excluded):

TABLE 2

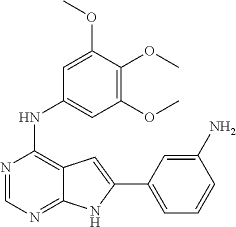 [SMILES: COc1cc(Nc2ncnc3[nH]c(-c4cccc(N)c4)cc23)cc(OC)c1OC]

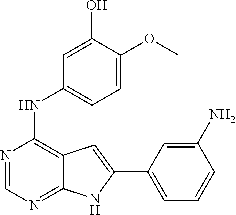 [SMILES: COc1ccc(Nc2ncnc3[nH]c(-c4cccc(N)c4)cc23)cc1O]

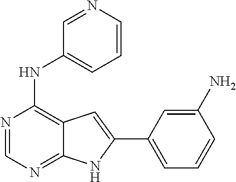 [SMILES: Nc1cccc(-c2cc3c(Nc4cccnc4)ncnc3[nH]2)c1]

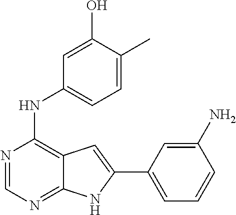 [SMILES: Cc1ccc(Nc2ncnc3[nH]c(-c4cccc(N)c4)cc23)cc1O]

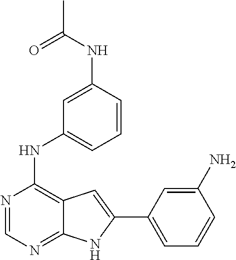 [SMILES: CC(=O)Nc1cccc(Nc2ncnc3[nH]c(-c4cccc(N)c4)cc23)c1]

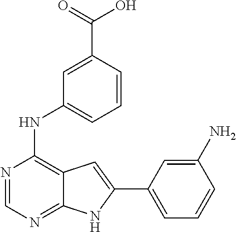 [SMILES: Nc1cccc(-c2cc3c(Nc4cccc(C(=O)O)c4)ncnc3[nH]2)c1]

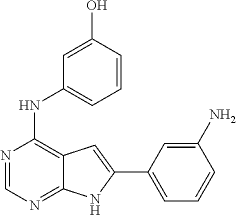 [SMILES: Nc1cccc(-c2cc3c(Nc4cccc(O)c4)ncnc3[nH]2)c1]

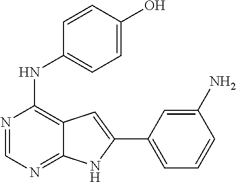 [SMILES: Nc1cccc(-c2cc3c(Nc4ccc(O)cc4)ncnc3[nH]2)c1]

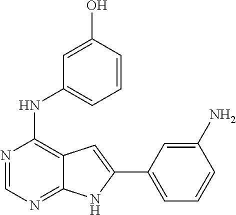 [SMILES: Nc1cccc(-c2cc3c(Nc4cccc(O)c4)ncnc3[nH]2)c1]

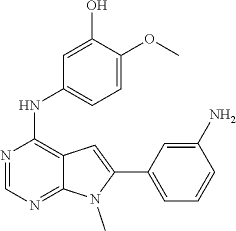 [SMILES: COc1ccc(Nc2ncnc3c2cc(-c2cccc(N)c2)n3C)cc1O]

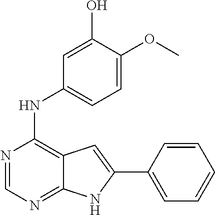 [SMILES: COc1ccc(Nc2ncnc3[nH]c(-c4ccccc4)cc23)cc1O]

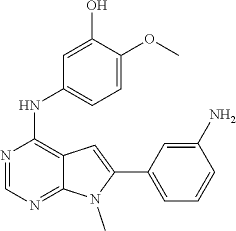 [SMILES: COc1ccc(Nc2ncnc3c2cc(-c2cccc(N)c2)n3C)cc1O]

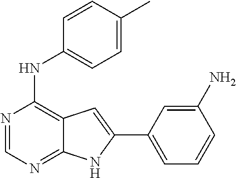 [SMILES: Cc1ccc(Nc2ncnc3[nH]c(-c4cccc(N)c4)cc23)cc1]

In a particular embodiment, the invention provides a compound of formula (I)

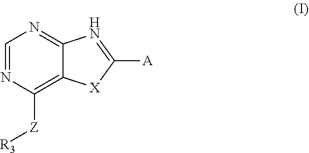

wherein:
   X is N or CH;
   Z is NR², O, S, or CHR², wherein R² is hydrogen, halogen (preferably F), methyl or ethyl, or deuterated or partially deuterated methyl or ethyl;
   A is a 5- or 6-membered hydrocarbyl or heterocyclic ring which may be optionally substituted with at least one R¹ group;
   each R¹ is independently selected from halogen, hydroxyl, —OCF₃, —CF₃, —CF₂H, —OCF₂H, CH(CF₃)OH, —C₁₋₆-alkyl, —O—C₁₋₆-alkyl, —O—(C₁-C₆ alkyl)-CH₂F, —O—(C₁-C₆ alkyl)-CHF₂, —O—(C₁-C₆ alkyl)-CF₃, —C₁₋₆alkyl-OH, —[C₁₋₆alkyl]ₘ-COOH, —[C₁₋₆alkyl]ₘ-COOC₁₋₆alkyl, —[C₁₋₆alkyl]ₘ-OCOC₁₋₆alkyl, —OCOR, —CO—[C₁₋₆alkyl]-COOH, —CO—[C₁₋₆alkyl]-COOR, C(CH₃)₂—CH₂OH, —C(OH)₂(C₁₋₆alkyl-OH), —C(OH)(C₁₋₆alkyl-OH)₂, —C(C₁₋₆alkyl-OH)₃, C(CH₃)₂OH, —NH₂, NHR, —NR₂, —NO₂, —SO₂NH₂, —NHSO₂R, —NRSO₂R, —SO₂NHR, SO₂NR₂, —POR₂, pyridyl (preferably ortho-pyridyl), cyclopropyl or cyclobutyl substituted with an —OH group, C₁-C₁₀ aliphatic hydrocarbyl group comprising at least one heteroatom selected from O, or N, a —O—C₁-C₁₀ aliphatic hydrocarbyl group containing at least one heteroatom selected from O or N, or a —[CH₂]ₘ-heterocycle wherein said heterocycle is optionally substituted with ═O or —OH;
   each m is 0 or 1;
   R³ is a 5- or 6-membered hydrocarbyl or heterocyclic ring either of which may be optionally substituted with one or more groups independently selected from C₁-C₄ alkyl, hydroxyl, halogen, —CF₃, —CF₂H, —NR₂, —NHCOR, —CO₂H, —CO₂R, or —OR; or R³ is a 5-membered hydrocarbyl ring fused with a 6-membered hydrocarbyl or heterocyclic ring, optionally substituted with one or more groups independently selected from C₁-C₄ alkyl, hydroxyl, halogen, —CF₃, —CF₂H, —NR₂, —NHCOR, —CO₂H, —CO₂R, or —OR;
   each R is C₁-C₆ alkyl;
or a pharmaceutically acceptable salt or solvate thereof;
   with the proviso that:
the compound of formula (I) is not one of the compounds listed in Table 2; or
a) when Z is NH, and A is phenyl substituted with a meta-NH₂ group, then R³ is not phenyl substituted with one of the following:
   meta-OH and para-OMe
   meta-OH and para-Me
   3,4,5, tri-methoxy (i.e. —OMe in both meta-positions and the para-position)
   pyridyl
   meta-(NH)(COMe)
   meta-COOH
   meta-OH
   para-OH
   para-Me, and
b) when Z is O, and A is phenyl substituted with a meta-NH₂ group, then R³ is not meta-OH.

### Synthesis

To prepare the compounds of the invention, a cross-coupling reaction (e.g. Suzuki-Miyaura) is typically used to couple the (optionally protected) A group to the core bicyclic structure.

In a particular aspect, the invention provides a process for the preparation of a compound as defined herein, wherein said process comprises the steps of: reacting a compound of formula:

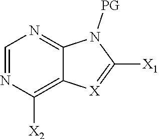

with
i) a compound of formula (RO)₂B-A or [A-BF₃]⁻ in the presence of a transition metal catalyst, wherein any OH groups in the R¹ substituent(s) of group A are optionally silane protected (e.g. with TBDMS), and
ii) a compound of formula —ZnBr—(CHR²)—R³ or —ZnBr—(CH₂)—R³ in the presence of a transition metal catalyst, or a compound of formula —OH—R³, —SH—R³, —(NHR²)—R³, or —(NH₂)—R³;
   in either order (i.e. i) then ii) or ii) then i));
then
iii) removing the protecting group PG;
   wherein
X₁ and X₂ are halogen;
PG is a protecting group, preferably selected from —SEM, THP, BOC, Cbz, Fmoc, SO₂Ph, Ts, MOM, CO₂H;
R is OH, OMe, or (RO)₂ is pinacol (i.e. —O—C(CH₃)₂—C(CH₃)₂—O—) or MIDA ester (i.e. —O—CO—CH₂—N(CH₃)—CH₂—CO—O—),
A, X, and R¹-R² are as defined above, below or in the claims.

In a particular embodiment, the process comprises an additional step of:
ii′) in the case of ZnBr—(CH₂)—R³ (i.e. if ZnBr—(CH₂)—R³ is used in step (ii)), reacting the resulting compound with a base followed by an alkylating agent (e.g. Mel or Etl) or fluorination agent (e.g. N-fluorobenzene-sulfonimide). Step (ii′) typically takes place after steps (i) and (ii). Alkylating agent also includes fluoroalkylating agents, e.g. for adding —CF₃.

In such a process, step i) may be carried out before step ii), or step ii) may be carried out before step i). Step iii) is always typically carried out after both steps i) and ii). Preferably the transition metal catalyst is a Pd-catalyst (e.g. PdCl₂dppf, Pd(PPh₃)₄, or Pd₂dba₃). Halogens X₁ and X₂ can be the same or different. Typically, they are different. In a particular embodiment, X₁ is I and X₂ is Cl. In a particular embodiment, the protecting group PG is SEM or THP. The deprotection step (removing the protecting group PG) can be achieved using any standard method, as will be known to the skilled chemist, e.g. using acid (p-TsOH, HCl, trifluoroacetic acid etc.).

MIDA esters are esters of N-methyliminodiacetic acid. SEM is —CH₂OCH₂CH₂SI(CH₃)₃. THP is tetrahydropyranyl. BOC is tert-butyloxycarbonyl. Cbz is carbobenzyloxy. Fmoc is 9-fluorenylmethyloxycarbonyl. Ts is tosyl. MOM is methoxymethyl ether. [A-BF₃]⁻ (i.e. organotrifluoroborates) may be presented as a salt with Li⁺, K⁺, or Na⁺, for example, preferably K⁺.

The starting material in the above embodiment can be prepared by the following reaction:

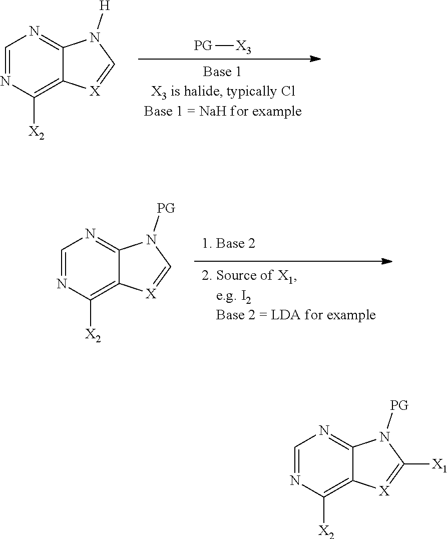

In other cases, the ‘PG’ group does not have to be presented as a reactant with a leaving group (e.g. X₃ above). For the protection of purines, for example, there is typically no leaving group. One can react with 3,4-dihydro-2H-pyran as shown below.

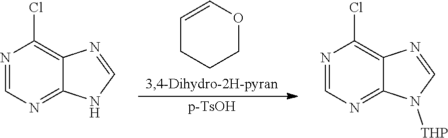

In a particular embodiment, the compounds of the invention can be prepared by the following routes

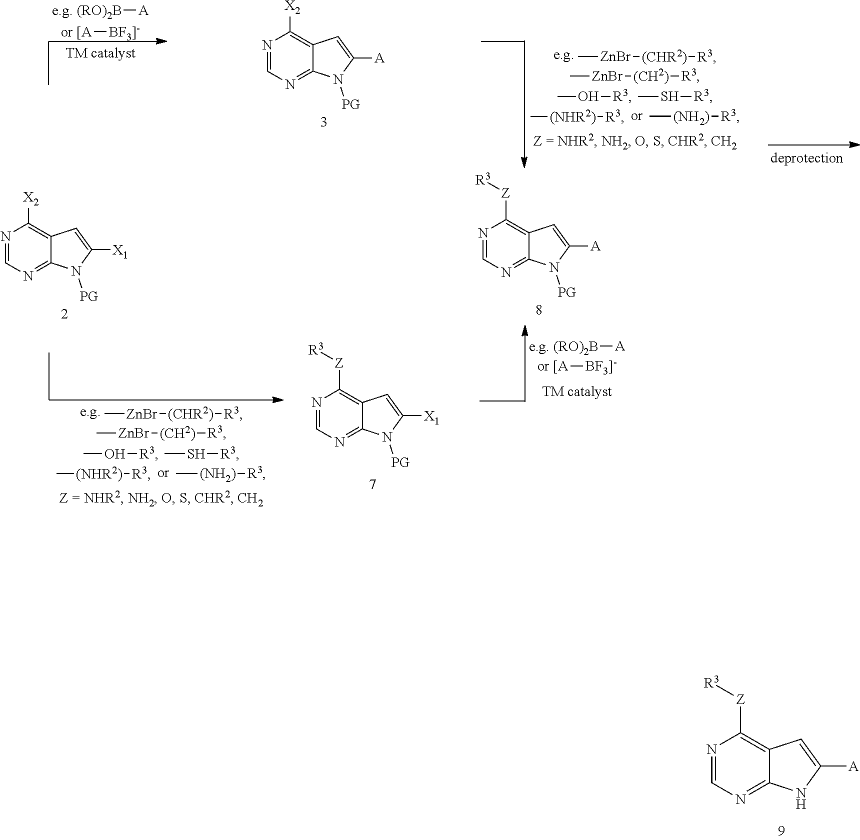

### Applications

The compounds of the present invention are potent inhibitors of CSF-1R. This results in the compounds of the invention being of particular interest in a number of therapeutic applications, given the broad role CSF-1R plays in a number of conditions.

In a particular aspect, the invention provides the compounds described herein for use in the treatment or prevention of a bone disorder, neurological disease, inflammatory disorder, cancer, or eye disease. The use may thus be therapeutic or prophylactic. Any discussion concerning the preventative/therapeutic uses or methods of the compounds of the invention apply equally to any pharmaceutical composition/formulation comprising the compounds of the invention.

The most preferred compounds of the invention typically offer CSF-1R IC₅₀ values of 65 nM or less, preferably 50 nM or less, preferably 28 nM or less, preferably 20 nM or less, preferably 15 nM or less, preferably 10 nM or less, such as 5 nM or less, 3 nM or less, or 1 nM or less. However, it is not a requirement for the compounds of the invention to have these IC₅₀ values. In a particular embodiment, compounds with IC₅₀ values above 10 nM, e.g. above 15 nM, e.g. above 20 nM, e.g. above 28 nM are excluded from the invention, e.g. those reported herein with IC50 values above these numbers.

In a particular embodiment, said bone disorder is osteoporosis, osteopetrosis, or osteosarcoma (for discussion, see Kodama, H. et al. Congenital osteoclast deficiency in osteopetrotic (op/op) mice is cured by injections of macrophage colony-stimulating factor. J. Exp. Med. 173, 269-272 (1991), and Smeester B A, Slipek N J, Pomeroy E J, Laoharawee K, Osum S H, Larsson A T, Williams K B, Stratton N, Yamamoto K, Peterson J J, Rathe S K, Mills L J, Hudson W A, Crosby M R, Wang M, Rahrmann E P, Moriarity B S, Largaespada D A. Bone. 2020 July; 136:115353. doi: 10.1016/j.bone.2020.115353). Preferably the bone disorder is osteoporosis.

In a particular embodiment, said neurological disease is selected from Charcot-Marie-Tooth disease, Alzheimer's disease, amyotrophic lateral sclerosis, traumatic brain injury, Hereditary diffuse leukoencephalopathy with spheroids.

In a particular embodiment, said inflammatory disorder is selected from rheumatoid arthritis and osteoarthritis.

In a particular embodiment, said cancer is selected from lung cancer, prostate cancer, colorectal cancer, stomach cancer, breast cancer, cervical cancer, multiple myeloma, ovary cancer, glioblastoma, breast cancer, malignant peripheral nerve sheath tumor, preferably multiple myeloma, ovary cancer, glioblastoma, breast cancer, malignant peripheral nerve sheath tumor.

In a particular embodiment, said eye disease is macular degeneration.

### Formulation

The compounds of the invention are preferably formulated as pharmaceutically acceptable compositions. The phrase “pharmaceutically acceptable”, as used in connection with compositions of the invention, refers to molecular entities and other ingredients of such compositions that are physiologically tolerable and do not typically produce untoward reactions when administered to a mammal (e.g. human). Preferably, as used herein, the term “pharmaceutically acceptable” means approved by a regulatory agency of the Federal or a state government or listed in the U.S. Pharmacopoeia or other generally recognized pharmacopoeia for use in mammals, and more particularly in humans.

The compounds of the invention can be administered in salt, solvate, prodrug or ester form, especially salt form. Typically, a pharmaceutical acceptable salt may be readily prepared by using a desired acid. The salt may precipitate from solution and be collected by filtration or may be recovered by evaporation of the solvent. For example, an aqueous solution of an acid such as hydrochloric acid may be added to an aqueous suspension of a compound of formula (I) and the resulting mixture evaporated to dryness (lyophilised) to obtain the acid addition salt as a solid. Alternatively, a compound of the invention may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

Suitable addition salts are formed from inorganic or organic acids which form non-toxic salts and examples are hydrochloride, hydrobromide, hydroiodide, sulphate, bisulphate, nitrate, phosphate, hydrogen phosphate, acetate, trifluoroacetate, maleate, malate, fumarate, lactate, tartrate, citrate, formate, gluconate, succinate, pyruvate, oxalate, oxaloacetate, trifluoroacetate, saccharate, benzoate, alkyl or aryl sulphonates (eg methanesulphonate, ethanesulphonate, benzenesulphonate or p-toluenesulphonate) and isethionate. Representative examples include trifluoroacetate and formate salts, for example the bis or tris trifluoroacetate salts and the mono or diformate salts, in particular the tris or bis trifluoroacetate salt and the monoformate salt.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compounds of the invention are within the scope of the invention. The salts of the compound of Formula (I) may form solvates (e.g. hydrates) and the invention also includes all such solvates.

The term “prodrug” as used herein means a compound which is converted within the body, e.g. by hydrolysis in the blood, into its active form that has medical effects.

The compounds of the invention are proposed for use in the treatment or prevention of a bone disorder, neurological disease, inflammatory disorder, cancer, or eye disease. By treating or treatment is meant at least one of:
(i). preventing or delaying the appearance of clinical symptoms of the disease developing in a mammal;
(ii). inhibiting the disease i.e. arresting, reducing or delaying the development of the disease or a relapse thereof or at least one clinical or subclinical symptom thereof, or
(iii). relieving or attenuating one or more of the clinical or subclinical symptoms of the disease.

The benefit to a subject to be treated is either statistically significant or at least perceptible to the patient or to the physician. In general a skilled man can appreciate when “treatment” occurs.

The word prevention is used herein to cover prophylactic treatment, i.e. treating subjects who are at risk of developing a disease in question.

The compounds of the invention can be used on any animal subject, in particular a mammal and more particularly to a human or an animal serving as a model for a disease (e.g. mouse, monkey, etc.).

An “effective dose” means the amount of a compound that, when administered to an animal for treating a state, disorder or condition, is sufficient to effect such treatment. The “effective dose” will vary depending on the compound, the disease and its severity and the age, weight, physical condition and responsiveness of the subject to be treated and will be ultimately at the discretion of the attendant doctor.

While it is possible that, for use in the methods of the invention, a compound of the invention may be administered as the bulk substance, it is preferable to present the active ingredient in a pharmaceutical formulation, for example, wherein the agent is in admixture with a pharmaceutically acceptable excipient or carrier selected with regard to the intended route of administration and standard pharmaceutical practice. All of the discussion about use in the treatment or prevention of various diseases thus also applies to the formulations of the invention. In a particular embodiment, therefore, the invention provides a pharmaceutical composition comprising a compound as defined herein and at least one excipient, for use in the treatment of a bone disorder, neurological disease, inflammatory disorder, cancer, or eye disease.

The term “excipient” refers to a diluent, carrier, and/or vehicle with which an active compound is administered. The pharmaceutical compositions of the invention may contain combinations of more than one excipient or carrier. Such pharmaceutical excipients or carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition. The choice of pharmaceutical carrier can be selected with regard to the intended route of administration and standard pharmaceutical practice. The pharmaceutical compositions may comprise as, in addition to, the excipient any suitable binder(s), lubricant(s), suspending agent(s), coating agent(s), and/or solubilizing agent(s). Particularly preferred for the present invention are carriers suitable for immediate-release, i.e., release of most or all of the active ingredient over a short period of time, such as 60 minutes or less, and make rapid absorption of the drug possible.

It will be appreciated that pharmaceutical compositions for use in accordance with the present invention may be in the form of oral, parenteral, transdermal, inhalation, sublingual, topical, implant, nasal, or enterally administered (or other mucosally administered) suspensions, capsules or tablets, which may be formulated in conventional manner using one or more pharmaceutically acceptable carriers or excipients.

There may be different composition/formulation requirements depending on the different delivery systems. Likewise, if the composition comprises more than one active component, then those components may be administered by the same or different routes.

The pharmaceutical formulations of the present invention can be liquids that are suitable for oral, mucosal and/or parenteral administration, for example, drops, syrups, solutions, injectable solutions that are ready for use or are prepared by the dilution of a freeze-dried product but are preferably solid or semisolid as tablets, capsules, granules, powders, pellets, pessaries, suppositories, creams, salves, gels, ointments; or solutions, suspensions, emulsions, or other forms suitable for administration by the transdermal route or by inhalation.

The compounds of the invention can be administered for immediate-, delayed-, modified-, sustained-, pulsed- or controlled-release applications.

In one aspect, oral compositions are slow, delayed or positioned release (e.g., enteric especially colonic release) tablets or capsules. This release profile can be achieved without limitation by use of a coating resistant to conditions within the stomach but releasing the contents in the colon or other portion of the GI tract wherein a site has been identified or a delayed release can be achieved by a coating that is simply slow to disintegrate or the two (delayed and positioned release) profiles can be combined in a single formulation by choice of one or more appropriate coatings and other excipients. Such formulations constitute a further feature of the present invention.

Pharmaceutical compositions can be prepared by mixing a therapeutically effective amount of the active substance with a pharmaceutically acceptable carrier that can have different forms, depending on the way of administration. Typically composition components include one or more of binders, fillers, lubricants, odorants, dyes, sweeteners, surfactants, preservatives, stabilizers and antioxidants.

The pharmaceutical compositions of the invention may contain from 0.01 to 99% weight—per volume of the active material. The therapeutic doses will generally be between about 10 and 2000 mg/day and preferably between about 30 and 1500 mg/day. Other ranges may be used, including, for example, 50-500 mg/day, 50-300 mg/day, 100-200 mg/day.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art.

It is within the scope of the invention for a compound as described herein to be administered in combination with another pharmaceutical, e.g. another drug with known efficacy against the disease in question. The compounds of the invention may therefore be used in combination therapy. ‘In combination’ here means in parallel; the other agents may be administered before, during, or after administration of the compound/formulation of the invention.

In particular, the compounds of the invention may be used in combination with other inhibitors with other targets, and other chemotherapeutic agents (cisplatin, taxol etc) used to treat cancerous diseases.

Also within the scope of the invention is the combination of the compounds of the invention with monoclonal antibodies.

In a particular embodiment, the compound of the invention is administered in combination with radiotherapy. ‘In combination’ here again means in parallel; the radiotherapy may be administered before, during, or after administration of the compound/formulation of the invention.

### Examples

### Core Scaffold

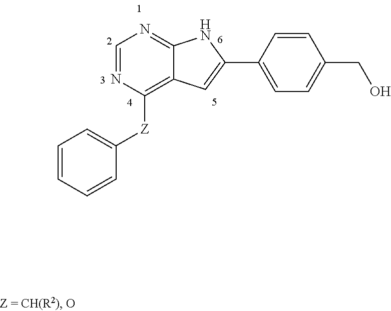

Core scaffold and its numbering system.

### Synthesis of Carbon Linked Structures

Low temperature iodination at C-6
Chemoselective Suzuki cross-coupling at C-6
Negishi cross-coupling at C-4
Deprotection of the SEM protection group (—CH₂—O—CH₂CH₂—Si(CH₃)₃

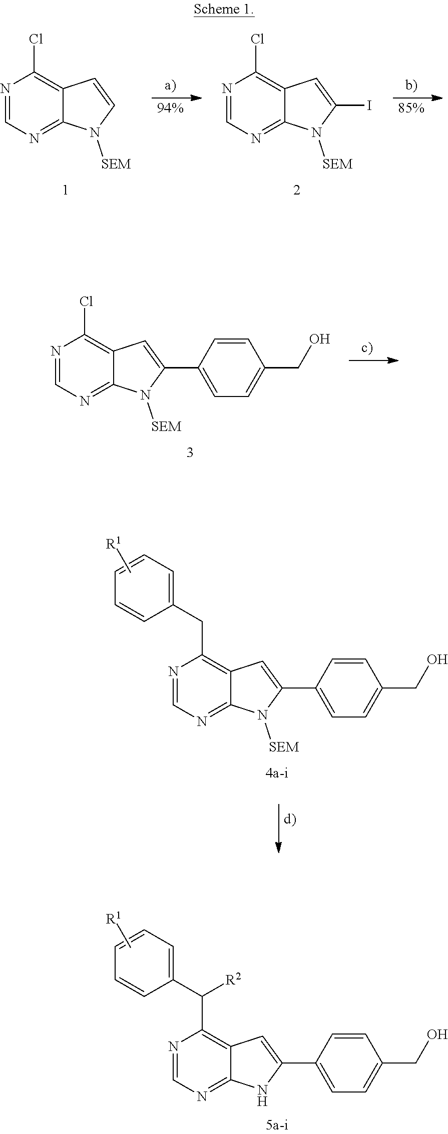

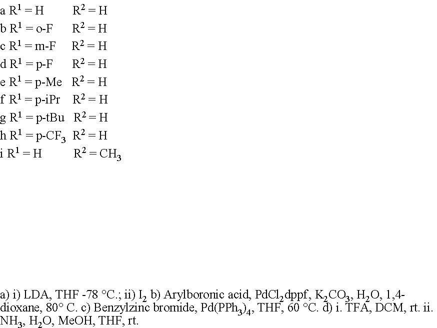

When the pyrrolopyrimine do not contain NH or OH functionality the benzylic carbon can be deprotonated and treated with various electrophiles.
Compound 6a was made either as shown in Scheme 1 or via intermediate 3 followed by Negishi cross-coupling
Treatment of 6a with lithium bis(trimethylsilyl)amide (LiHMDS) gives a stabilized anion which was reacted with two alkylating agents and one fluorination agent.
Deprotection of the SEM group and TBDMS groups gave compounds 5i-k (Scheme 2).

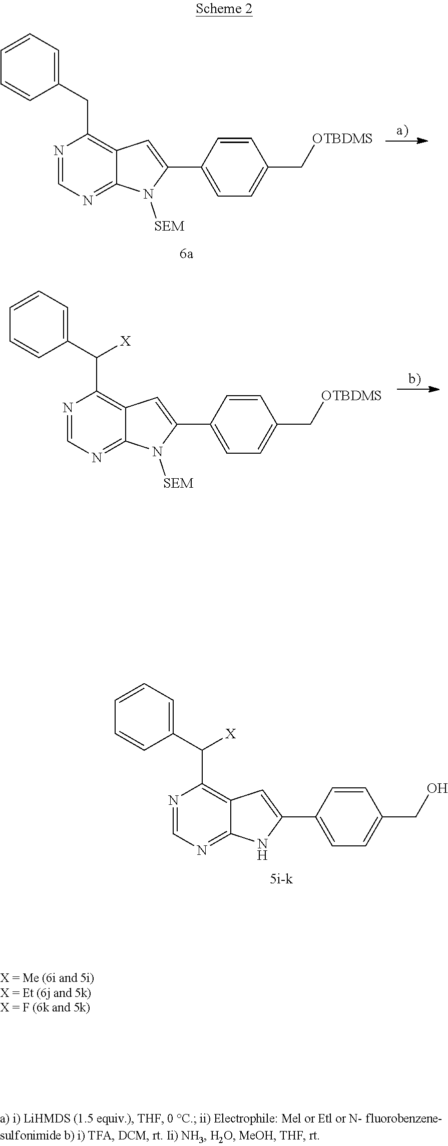

### Synthesis Towards Oxygen Linked Structures:

Two routes were used

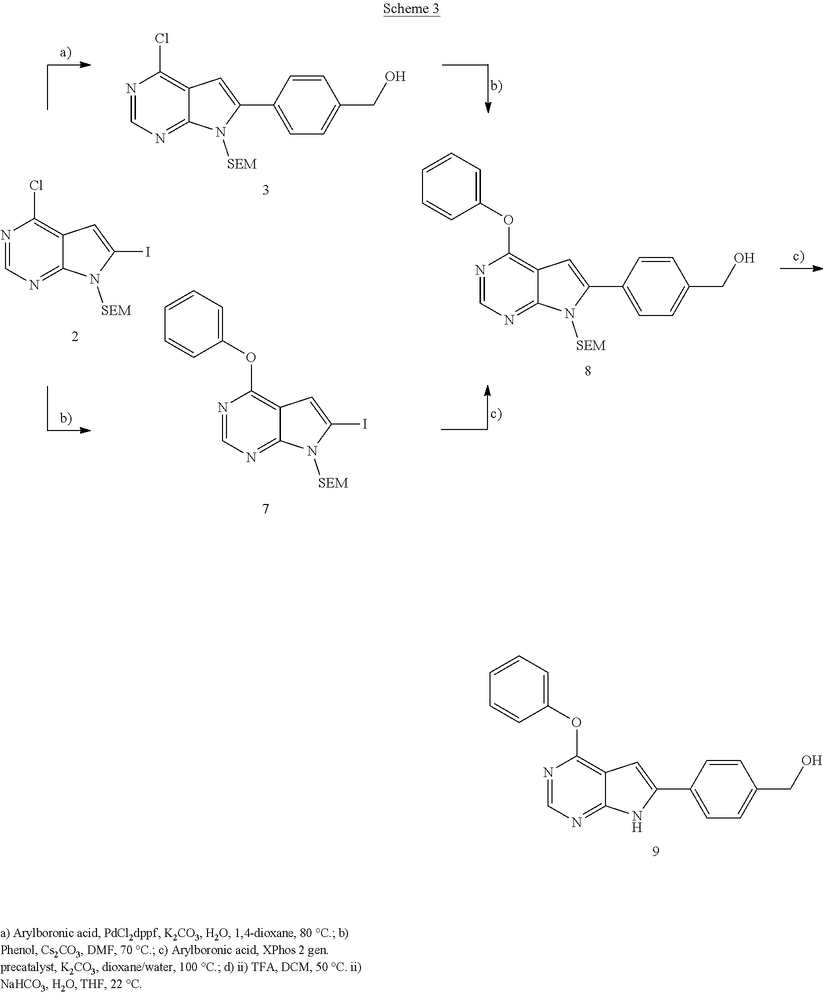 [SMILES: C[Si](C)(C)CCOCn1c(I)cc2c(Cl)ncnc21 ; C[Si](C)(C)CCOCn1c(-c2ccc(CO)cc2)cc2c(Cl)ncnc21 ; C[Si](C)(C)CCOCn1c(I)cc2c(Oc3ccccc3)ncnc21 ; C[Si](C)(C)CCOCn1c(-c2ccc(CO)cc2)cc2c(Oc3ccccc3)ncnc21 ; OCc1ccc(-c2cc3c(Oc4ccccc4)ncnc3[nH]2)cc1]

### Biological Data

TABLE 3a
Structures of compounds 5a-k, 9 and 10 with half-maximal inhibitory
concentrations against CSF1R enzyme and CSF1R in bone-marrow derived
macrophages from mice.

| | | | |
|---|---|---|---|
| | 5a-k and 9 | | 10 |
| 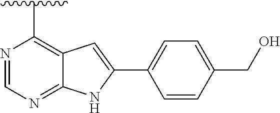 [SMILES: *c1ncnc2[nH]c(-c3ccc(CO)cc3)cc12] | | 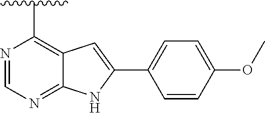 [SMILES: *c1ncnc2[nH]c(-c3ccc(OC)cc3)cc12] | |

| | | CSF1R | | BMDM |
|---|---|---|---|---|
| | | Z-lyte | BMDM | fold |
| | | IC₅₀ | IC₅₀ | relative to |
| Compound | R | (nM)a) | [nM] | PLX3397 |
| 5a (TIA03-170) | 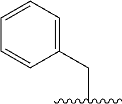 [SMILES: *Cc1ccccc1] | 1.3 | 130^{a)} | 1 |
| 5b (VT-44-03) | 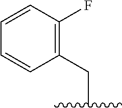 [SMILES: *Cc1ccccc1F] | 0.7 | 487 ^{b)} | 5 |
| 5c (VT-84-06) | 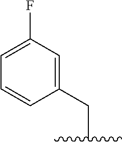 [SMILES: *Cc1cccc(F)c1] | 1.9 | >500 ^{b)} | >5 |
| 5d (VT-22-03) | 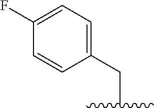 [SMILES: *Cc1ccc(F)cc1] | 3.1 | 320 ^{a)} | 4.1 |
| 5e (VT-64-03) | 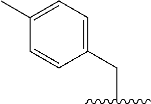 [SMILES: *Cc1ccc(C)cc1] | 1.0 | >300 ^{b)} | >3 |
| 5f(VT-82-03) | 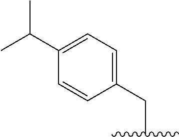 [SMILES: *Cc1ccc(C(C)C)cc1] | 0.6 | 71^{b)} | 0.8 |
| 5g (VT-28-02) | 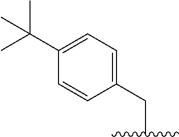 [SMILES: *Cc1ccc(C(C)(C)C)cc1] | 1.6 | 174 ^{a)} | 1.7 |
| 5h (VT-56-03) | 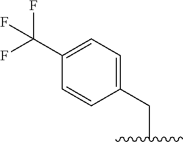 [SMILES: *Cc1ccc(C(F)(F)F)cc1] | 3.6 | >500 ^{b)} | >5 |
| 5i (VT-12-03; VT-10-05) | 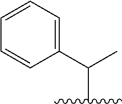 [SMILES: *C(C)c1ccccc1] | 3.8 | >500 ^{a)} | >5 |
| 5j (TIA05-134) | 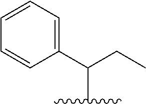 [SMILES: *C(CC)c1ccccc1] | 29 | >500 ^{a)} | >5 |
| 5k (TIA05-136) | 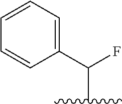 [SMILES: *C(F)c1ccccc1] | 2.7 | 100 ^{a)} | 1.3 |
| 9 (HIL120) | 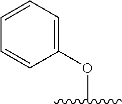 [SMILES: *Oc1ccccc1] | 1.5 | 107 | 1 |
| 10 (TIA03-164) | 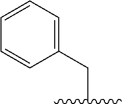 [SMILES: *Cc1ccccc1] | 11 | ND | ND |
| PLX5622 | Positive control | 19 | ND | ND |
| PLX3397 | Positive control | 7.2 | 70-150 | 1 |

^{a)} Enzymatic IC₅₀ values obtained by 10-point titrations in duplicates (20 data points) using Z-LYTE assay platform.
^{b)} Assayed in parallel with PLX3397. IC₅₀ based on 5 concentrations, three parallels and two replicates.
^{c)} Assayed in parallel with PLX3397. IC₅₀ based on 5 concentrations and three parallels

### PLX-3397 has the Following Structure:

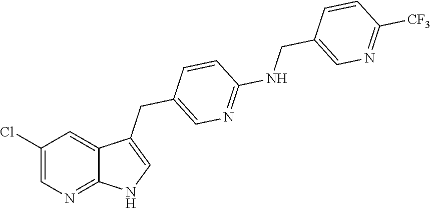 [SMILES: FC(F)(F)c1ccc(CNc2ccc(Cc3c[nH]c4ncc(Cl)cc34)cn2)cn1]

### PLX5622 has the Following Structure

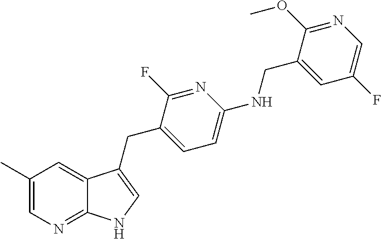 [SMILES: COc1ncc(F)cc1CNc1ccc(Cc2c[nH]c3ncc(C)cc23)c(F)n1]

### Biological Assays

TABLE 3b
Structures of additional pyrrolopyrimidine compounds with half-maximal
inhibitory concentrations against CSF1R enzyme and CSF1R in bone-marrow
derived macrophages from mice.

| | | CSF1R | | BMDM |
|---|---|---|---|---|
| | | Z-lyte | BMDM | fold |
| | | IC₅₀ | IC₅₀ | relative to |
| Compound | Structure | (nM)a) | [nM] | PLX3397 |
| 11 SH-01-81 | 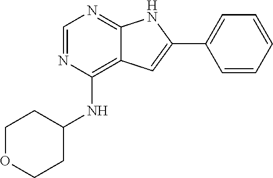 [SMILES: c1ccc(-c2cc3c(NC4CCOCC4)ncnc3[nH]2)cc1] | 60 | ND | ND |
| 12 SH-01-98 | 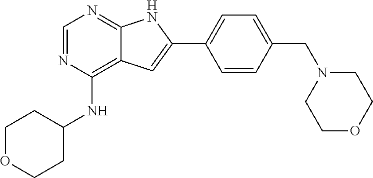 [SMILES: c1nc(NC2CCOCC2)c2cc(-c3ccc(CN4CCOCC4)cc3)[nH]c2n1] | 14 | ND | ND |
| 14 LR-2-144 | 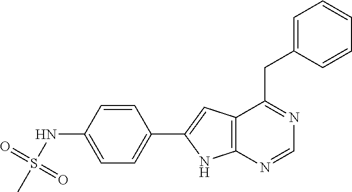 [SMILES: CS(=O)(=O)Nc1ccc(-c2cc3c(Cc4ccccc4)ncnc3[nH]2)cc1] | 2.1 | ND | ND |
| 15 LR-2-163 | 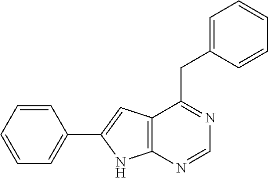 [SMILES: c1ccc(Cc2ncnc3[nH]c(-c4ccccc4)cc23)cc1] | 7.2 | ND | ND |
| 16 LR-2-177 | 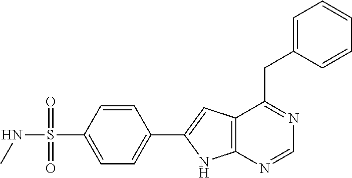 [SMILES: CNS(=O)(=O)c1ccc(-c2cc3c(Cc4ccccc4)ncnc3[nH]2)cc1] | 1.7 | ND | ND |
| 17 LR-2-179 | 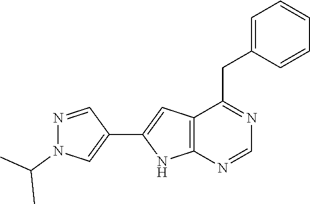 [SMILES: CC(C)n1cc(-c2cc3c(Cc4ccccc4)ncnc3[nH]2)cn1] | 2.0 | ND | ND |
| 18 LR-2-185 | 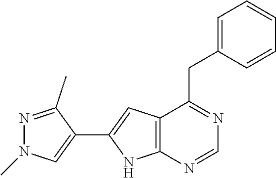 [SMILES: Cc1nn(C)cc1-c1cc2c(Cc3ccccc3)ncnc2[nH]1] | 0.4 | ND | ND |
| 19 LR-3-002 | 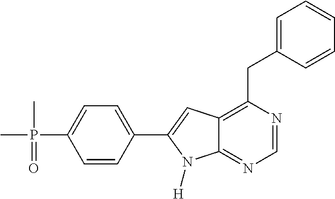 [SMILES: CP(C)(=O)c1ccc(-c2cc3c(Cc4ccccc4)ncnc3[nH]2)cc1] | 1.5 | ND | ND |

### CSF1R Enzymatic Inhibitory Assay

The compounds were supplied in a 10 mM DMSO solution, and enzymatic CSF1R inhibition potency was determined by Invitrogen (TermoFisher) using their Z′-LYTE® assay technology[2]. The assay is based on fluorescence resonance energy transfer (FRET). In the primary reaction, the kinase transfers the gamma-phosphate of ATP to a single tyrosine residue in a synthetic FRET-peptide. In the secondary reaction, a site-specific protease recognizes and cleaves non-phosphorylated FRET-peptides. Thus, phosphorylation of FRET-peptides suppresses cleavage by the development reagent. Cleavage disrupts FRET between the donor (i.e. coumarin) and acceptor (i.e., fluorescein) fluorophores on the FRET-peptide, whereas uncleaved, phosphorylated FRET-peptides maintain FRET. A ratiometric method, which calculates the ratio (the emission ratio) of donor emission to acceptor emission after excitation of the donor fluorophore at 400 nm, is used to quantitate inhibition. All compounds were first tested for their inhibitory activity at 500 nM in duplicates. The potency observed at 500 nM was used to set starting point of the IC50 titration curve, in which three levels were used 1000 or 10000 nM. The IC50 values reported are based on the average of at least 2 titration curves (minimum 20 data points), and were calculated from activity data with a four parameter logistic model using SigmaPlot (Windows Version 12.0 from Systat Software, Inc.) Unless stated otherwise the ATP concentration used was equal to Km (ca 10 mM).

### Effect of CSF1R Inhibitors on MAPK Signalling in Mouse Bone-Marrow Derived Macrophages

Bone-marrow derived macrophages were obtained by flushing the femur and tibia of sacrificed mice with HBSS (Hanks' balanced salt solution) using a syringe with a 25 G needle. The cells were centrifuged at 1500 rpm for 8 minutes, the resulting supernatant was decanted, and the cells resuspended in 5 mL RBC (red blood cell) lysis buffer. Lysis was stopped by adding 30 ml medium containing 10% FCS (Fetal Calf Serum). The cells were centrifuged at 1500 rpm for 8 minutes. The supernatant was decanted, and the cells resuspended in RPMI medium gentamycin, 2 mM glutamine, 10% FCS) with 10 ng/mL CSF-1 (colony stimulating factor 1). The cells were seeded in bacteria plates. After two days, fresh medium with 10 ng/mL CSF-1 was added and after another two days, 50% of the medium was replaced with fresh medium containing 10 ng/mL CSF-1 while the other 50% is centrifuged to get rid of dead cells before being transferred back to the cells. After incubating for one week, the differentiated cells were washed twice with PBS, added PBS EDTA (0.2 mM) and incubated for 10 minutes. Cells were detached by scraping and centrifuged at 1200 rpm for 7 minutes. The supernatant was decanted, and the cells resuspended in RPMI medium (10% FCS, 10 ng/ml CSF-1, x gentamycin, 2 mM L-glutamine). The cells were seeded out in 96-well glass bottom plates (Cellvis) at 50.000 cells in 100 μL per well and incubated at 37° C. overnight. The medium was removed, and the cells washed three times with PBS before being starved overnight in 0.1% FCS medium without CSF-1. CSF-1 inhibitors dissolved in DMSO, was added to the wells in appropriate concentrations and incubated for 30 minutes at 37° C. DMSO was added to control wells at the highest inhibitor concentration. CSF-1 (0.1 mg/mL) was added to all wells except the CSF-1 negative control to obtain an end concentration of CSF-1 of 10 ng/mL. After incubating for another 10 minutes at 37° C., the cells were fixed by adding PFA (paraformaldehyde) (16%) to obtain an end concentration of 4% for 10 minutes. The cells was washed twice with TBS, and permabilized by MeOH for 10 minutes on ice. The cells were washed twice with TBS and blocked in Odyssey blocking solution (Licor, #927-60001) diluted 1:1 in TBS-Tween (0.1%) for 1.5 hours under careful agitation. The blocking solution was removed and the wells added appropriately diluted primary antibody solution (P-MAPK (ERK1/2, Thr202/Tyr204) (CST, #4370, rabbit) 1:1200 and MAPK (ERK1/2), (Biolegend, #686902, rat) 1:300 in in Odyssey blocking buffer:TBS-Tween (1:1)). After incubating overnight with careful shaking at 4° C., the cells were washed with TBST 5 times for 5 minutes while agitating. Secondary antibody solution was added, and the wells were incubated for 1 hour in the dark Rdye 8000W goat anti-rabbit and IRdye 680RD goat anti-rat diluted 1:800 in Odyssey blocking buffer:TBS-Tween (1:1)). The antibody solution was removed, and the wells were washed 4×5 minutes with TBST and 2×5 minutes with TBS while carefully agitating. The TBS was removed and the plate was scanned on a Licor Odyssey scanner. Using Image Studio software, the intensity of fluorescence of each well is recorded after subtracting the background noise (wells not added primary antibody). The results were normalized by dividing the P-MAPK intensity with total MAPK intensity for all the wells. The average value of triplicate wells was calculated for every concentration of inhibitor used. The average values are then divided by the CSF-1 positive control value. PLX3397 was included as a reference on all plates. Due to some inter-assay variation the activity of the inhibitors is reported as fold change relative to PLX3397 (Fold change: IC50.inhibitor/IC50 PLX3397)

### Synthesis and Spectroscopic Data

### Intermediates

### 4-Chloro-6-iodo-7-((2-(trimethylsilyl)-ethoxy)methyl)-7H-pyrrolo[2,3-d]pyrimidine (2)

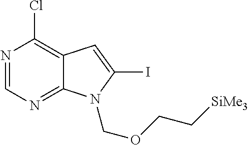 [SMILES: C[Si](C)(C)CCOCn1c(I)cc2c(Cl)ncnc21]

Under a N₂ atmosphere, 4-chloro-7-((2-(trimethylsilyl)-ethoxy)methyl)-7H-pyrrolo[2,3-d]pyrimidine (5.00 g, 17.6 mmol) was dissolved in dry THF (70 mL) and cooled down to −78° C. Then, LDA (2 M in THF/n-hexane/ethylbenzene, 13.3 mL, 26.6 mmol) was added dropwise over 30 min. This was followed by dropwise addition of I₂ (5.08 g, 20.1 mmol) dissolved in THF (12 mL). After another 30 min, the reaction mixture was quenched with saturated NH₄Cl solution (0.5 mL) and stirred until ambient temperature was reached. The mixture was concentrated and diluted with 10% Na₂S₂O₃ solution (20 mL), CH₂Cl₂ (25 mL) and water (30 mL). After phase separation, the water phase was extracted with more CH₂Cl₂ (4×20 mL). The combined organic phase was dried over Na₂SO₄ and the solvent was removed under reduced pressure. The crude product was purified by silica-gel flash column chromatography (n-pentane/EtOAc—9:1, R_{f}=0.44) giving 6.60 g (16.1 mmol, 92%) of 4-chloro-6-iodo-7-((2-(trimethylsilyl)-ethoxy)methyl)-7H-pyrrolo[2,3-d]pyrimidine as a grey powder, mp. 99-101° C.; ¹H NMR (600 MHz, DMSO-d₆) δ 8.62 (s, 1H), 7.11 (s, 1H), 5.61 (s, 2H), 3.53, (t, J=7.9 Hz, 2H), 0.82 (t, J=7.9 Hz, 2H), −1.11 (s, 9H); ¹³C NMR (150 MHz, DMSO-d₆) δ 152.5, 150.8, 149.0, 118.6, 109.8, 91.5, 73.5, 66.0, 17.1, −1.4 (3C).

### General Procedure A: Chemoselective Suzuki Cross-Coupling

4-Chloro-6-iodo-7-((2-(trimethylsilyl)-ethoxy)methyl)-7H-pyrrolo[2,3-d]pyrimidine (1.0 equiv.), boronic acid (1.0-1.2 equiv.), palladium catalyst (PdCl₂dppf or Pd₂dba₃) (2-5 mol %) and potassium carbonate (3.0 equiv.) is charged in an appropriate reaction vessel. The atmosphere is evacuated and back-filled with N₂ three times before adding degassed 1,4-dioxane (6 mL/mmol starting material) and degassed water (3 mL/mmol starting material). The reaction vessel is lowered into an oil-bath set at 60-80° C. and stirred vigorously. Upon reaction completion, the reaction vessel is raised from the oil-bath and allowed to cool for 5 min before the reaction mixture is transferred to a round-bottomed flask and the volatiles are removed by rotary evaporation. The residue is added water (20 mL/mmol starting material) and extracted with CH₂Cl₂ (3×20 mL/mmol starting material). The combined organic layers are washed with brine (20 mL/mmol), dried over anhydrous Na₂SO₄ and filtered. The organic solvent is removed under reduced pressure and the crude product is purified by silica-gel column chromatography.

### (4-(4-Chloro-7-((2-(trimethylsilyl)ethoxy)methyl)-7H-pyrrolo[2,3-d]pyrimidin-6-yl)phenyl)methanol (3)

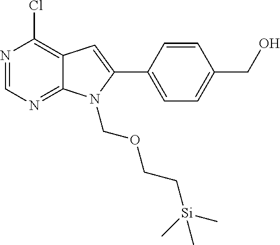 [SMILES: C[Si](C)(C)CCOCn1c(-c2ccc(CO)cc2)cc2c(Cl)ncnc21]

¹H NMR (400 MHz, CDCl₃) δ 8.67 (s, 1H), 7.79 (d, J=8.3, 2H), 7.52 (d, J=8.3 Hz, 2H), 6.71 (s, 1H), 5.62 (s, 2H), 4.80 (d, J=5.9 Hz, 2H), 3.76-3.72 (m, 2H), 1.84-1.82 (m, 1H), 0.99-0.95 (m, 2H), −0.02 (s, 9H); ¹³C NMR (100 MHz, CDCl₃) δ 153.5, 151.5, 150.9, 143.4, 142.2, 129.8 (2C), 129.7, 127.3 (2C), 117.6, 99.5, 71.1, 64.9, 18.0, −1.4 (3C); HRMS (ASAP+, m/z): found 390.1405, calcd for C₁₉H₂₅N₃O₂SiCl, [M+H]⁺, 390.1405.

### 4-Chloro-6-(4-methoxyphenyl)-7-((2-(trimethylsilyl)ethoxy)methyl)-7H-pyrrolo[2,3-d]pyrimidine

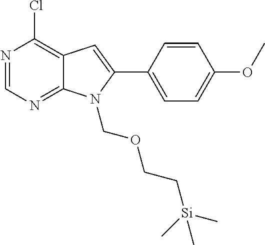 [SMILES: COc1ccc(-c2cc3c(Cl)ncnc3n2COCC[Si](C)(C)C)cc1]

¹H NMR (400 MHz, DMSO-d₆) δ: 8.69 (s, 1H), 7.78-7.76 (m, 2H), 7.12-7.10 (m, 2H), 6.80 (s, 1H), 5.61 (s, 2H), 3.84 (s, 3H), 3.63-3.59 (m, 2H), 0.87-0.83 (m, 2H), −0.10 (s, 9H); ¹³C NMR (100 MHz, DMSO-d₆) δ: 160.22, 152.97, 150.48, 149.85, 143.46, 130.61 (2C), 122.27, 116.87, 114.44 (2C), 98.05, 70.94, 66.19, 55.36, 17.26, −1.46 (3C); HRMS (ASAP+, m/z): found 390.1402, calcd for C₁₉H₂₅N₃O₂SiCl [M+H]⁺ 390.1405.

### 6-(4-(((Tert-butyldimethylsilyl)oxy)methyl)phenyl)-4-chloro-7-((2-(trimethylsilyl)ethoxy)methyl)-7H-pyrrolo[2,3-d]pyrimidine

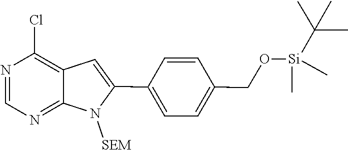 [SMILES: CC(C)(C)[Si](C)(C)OCc1ccc(-c2cc3c(Cl)ncnc3n2COCC[Si](C)(C)C)cc1]

Compound 3 (460 mg, 1.180 mmol) and imidazole (121 mg, 1.77 mmol) were dissolved in THF (1.5 mL), and a solution of TBDMS-CI (213 mg, 1.41 mmol) in THF (1.0 mL) was added dropwise to the mixture over 5 min. The reaction mixture was stirred at rt for 1 h before removing the volatiles in vacuo. The crude reaction mixture was partitioned between CH₂Cl₂ (10 mL) and water (10 mL), the layers were separated and the aqueous phase was extracted with CH₂Cl₂ (2×10 mL). The combined organic phases were washed with water (10 mL) and brine (10 mL), dried over anhydrous Na₂SO₄, filtered and concentrated in vacuo. The crude product was purified by silica-gel column chromatography (n-pentane/EtOAc—93:7, R_{f}=0.26) giving 526 mg (1.044 mmol, 88%) of a colorless solid, mp. 69.2-70.8° C. ¹H NMR (400 MHz, CDCl₃) δ 8.67 (s, 1H), 7.78-7.71 (m, 2H), 7.50-7.43 (m, 2H), 6.69 (s, 1H), 5.62 (s, 2H), 4.83 (s, 2H), 3.78-3.69 (m, 2H), 1.00-0.95 (m, 9H), 0.97 (s, 2H), 0.14 (s, 6H), −0.03 (s, 9H); ¹³C NMR (101 MHz, CDCl₃) δ 153.4, 151.4, 150.8, 143.8, 143.0, 129.4 (2C), 129.1, 126.4 (2C), 117.7, 99.2, 71.1, 67.1, 64.6, 26.0 (3C), 18.5, 18.0, −1.4 (3C), −5.2 (2C); HRMS (ES+, m/z): found 504.2265 calcd for C₂₅H₃₉ClN₃O₂Si₂ [M+H]⁺ 504.2264.

### General Procedure B: Negishi Cross-Coupling

Preparation of benzylzinc bromides: Zn (6 mmol) suspended in anhydrous THF (3.5 mL) was activated by adding 1,2-dibromoethane (25 μL) and stirring at reflux for 10 min. Trimethylsilyl chloride (10 μL) was added and the mixture was stirred for another 5 min. at reflux before being cooled down to rt. Benzyl bromide (5 mmol) dissolved in dry THF (1.5 mL) was added dropwise to the mixture over 1 h. The reaction mixture was stirred for 3 h and subsequently stored refrigerated (1-4° C.) overnight under an N₂-atmosphere. The concentration of the resulting benzylzinc bromide solution was determined by titration into a solution of I₂ (50.8 mg, 0.2 mmol) dissolved in LiCl in THF (1.5 mL, 0.5 M) at 0° C.

Negishi cross-coupling: The pyrrolopyrimidine 3 (1.03 mmol, 1 equiv.) and Pd(PPh₃)₄ (57.6 mg, 0.05 mmol, 1 equiv.) were dissolved in dry THF (6 mL/mmol aryl chloride) and the benzylzinc bromide solution (0.5-1.0 M in THF, 3.09 mmol, 3 equiv.) under an N₂-atmosphere. The reaction mixture was stirred at 60° C. for 1-3 h before it was concentrated in vacuo. The residue was added water (25 mL) or aqueous ammonia (25 mL, 5-10%) and extracted with CH₂Cl₂ (3×25 mL) or EtOAc (3×25 mL).

In some experiments quenching was done using water containing 6% by vol. of ammonia. The combined organic phases were washed with brine (50 mL), dried over Na₂SO₄, filtered and concentrated in vacuo. The crude product was purified by silica-gel column chromatography as specified for each compound.

### (4-(4-Benzyl-7-((2-(trimethylsilyl)ethoxy)methyl)-7H-pyrrolo[2,3-d]pyrimidin-6-yl)phenyl)methanol (4a)

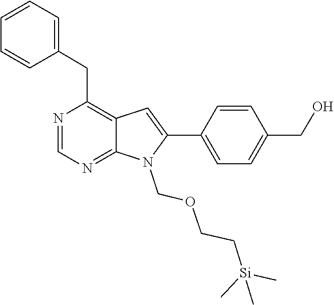 [SMILES: C[Si](C)(C)CCOCn1c(-c2ccc(CO)cc2)cc2c(Cc3ccccc3)ncnc21]

Compound 3 (390 mg, 1.01 mmol) was treated as described in General Procedure B with benzylzinc bromide (3.08 mmol in 2.99 mL THF). Purification by silica-gel column chromatography (n-pentane/EtOAc—1:1, R_{f}=0.26) gave 396 mg (0.889 mmol, 88%) of an off-white solid, mp. 63.1-64.6° C.; ¹H NMR (600 MHz, CDCl₃) δ 8.84 (s, 1H), 7.72 (d, J=8.3 Hz, 2H), 7.48 (d, J=8.3 Hz, 2H), 7.36 (d, J=7.3 Hz, 2H), 7.29 (d, J=7.3 Hz, 2H), 7.23-7.19 (m, 1H), 6.51 (s, 1H), 5.59 (s, 2H), 4.77 (d, J=3.8, 2H), 4.39 (s, 2H), 3.75-3.71 (m, 2H), 2.12 (s, 1H), 0.97-0.93 (m, 2H), −0.04 (s, 9H); ¹³C NMR (100 MHz, CDCl₃) δ 160.5, 153.3, 151.7, 142.2, 141.8, 138.1, 130.4, 129.6 (2C), 129.1 (2C), 128.6 (2C), 127.2 (2C), 126.7, 117.5, 99.5, 70.7, 66.9, 64.8, 42.3, 18.0, −1.4 (3C); HRMS (ASAP+, m/z): found 446.2264, calcd for C₂₆H₃₂N₃O₂Si [M+H]⁺ 446.2264.

### (4-(4-(2-Fluorobenzyl)-7-((2-(trimethylsilyl)ethoxy)methyl)-7H-pyrrolo[2,3-d]pyrimidin-6-yl)phenyl)methanol (4b)

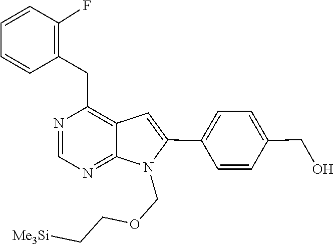 [SMILES: C[Si](C)(C)CCOCn1c(-c2ccc(CO)cc2)cc2c(Cc3ccccc3F)ncnc21]

Compound 3 (199 mg, 0.510 mmol) was treated as described in General Procedure B with 2-fluorobenzylzinc bromide (1.71 mL, 1.57 mmol, 0.92 M in THF). Quenching was done with water (20 mL cont. 6% NH₃) followed by extraction with EtOAc (3×20 mL). Purification by silica-gel column chromatography (CH₂Cl₂/EtOAc—5:2, R_{f}=0.20) gave 202 mg (0.435 mmol, 85%) of an off-white solid, mp. 97.5-98.0° C.; ¹H NMR (600 MHz, CDCl₃) δ 8.82 (s, 1H), 7.72 (d, J=8.2 Hz, 2H), 7.48 (d, J=8.2 Hz, 2H), 7.34 (td, J=7.6 Hz, 1.5 Hz, 2H), 7.23-7.19 (m, 1H), 7.07-7.03 (m, 2H), 6.54 (s, 1H), 5.58 (s, 2H), 4.77 (s, 1H), 4.41 (s, 2H), 3.74-3.72 (m, 2H), 2.72 (s, 1H), 0.97-0.94 (m, 2H), −0.04 (s, 9H); ¹³C NMR (150 MHz, CDCl₃) δ 160.8 (d, J=246.5 Hz, 1C), 159.3, 153.2, 151.5, 142.3, 142.0, 131.4 (d, J=4.4 Hz, 1C), 130.3, 129.5 (2C), 128.6 (d, J=8.5 Hz), 127.2 (2C), 125.0 (d, J=15.6 Hz), 124.2 (d, J=3.4 Hz), 117.5, 115.4 (d, J=22.3 Hz), 99.4, 70.7, 66.8, 64.7, 34.7 (d, J=3.2 Hz), 18.0, −1.4 (3C); ¹⁹F-NMR (376 MHz, CDCl₃, C₆F₆): −120.2; HRMS (ASAP+, m/z): found 464.2173, calcd for C₂₆H₃₁N₃O₂FSi [M+H]⁺ 464.2170.

### (4-(4-(3-Fluorobenzyl)-7-((2-(trimethylsilyl)ethoxy)methyl)-7H-pyrrolo[2,3-d]pyrimidin-6-yl)phenyl)methanol (4c)

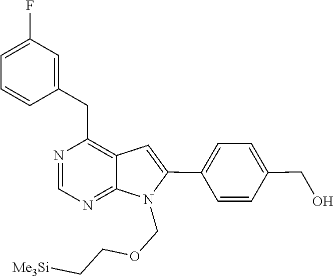 [SMILES: C[Si](C)(C)CCOCn1c(-c2ccc(CO)cc2)cc2c(Cc3cccc(F)c3)ncnc21]

Compound 3 (197 mg, 0.505 mmol) was treated as described in General Procedure B with 3-fluorobenzylzinc bromide (1.68 mL, 1.03 mmol, 0.61 M in THF). Quenching was done with water (20 mL cont. 6% NH₃) followed by extraction with EtOAc (3×20 mL). Purification by silica-gel column chromatography (CH₂Cl₂/EtOAc—4:1, R_{f}=0.22) gave 91 mg (0.096 mmol, 39%) of an off-white solid, mp. 118.5-119.0° C.; ¹H NMR (600 MHz, CDCl₃) δ 8.85 (s, 1H), 7.74 (d, J=8.2 Hz, 2H), 7.49 (d, J=8.2 Hz, 2H), 7.27-7.23 (m, 1H), 7.13 (d, J=7.6 Hz, 1H), 7.04 (d, J=9.8 Hz, 1H), 6.91 (td, J=8.5 and 2.4 Hz, 1H), 6.52 (s, 1H), 5.60 (s, 2H), 4.79 (d, J=5.8 Hz, 2H), 4.38 (s, 2H), 3.75-3.73 (m, 2H), 1.88 (t, J=5.8 Hz, 1H), 0.97-0.95 (m, 2H), −0.04 (s, 9H); ¹³C NMR (150 MHz, CDCl3) δ 162.9 (d, J=245.7 Hz), 159.6, 153.3, 151.7, 142.4, 141.8, 140.5 (d, J=7.6 Hz), 130.3, 130.0 (d, J=8.7 Hz), 129.6 (2C), 127.2 (2C), 124.8 (d, J=3.0 Hz), 117.5, 116.0 (d, J=21.2 Hz), 113.6 (d, J=21.2 Hz), 99.3, 70.7, 66.9, 64.9, 41.8, 18.0, −1.4 (3C); ¹⁹F-NMR (376 MHz, CDCl₃, C₆F₆): −116.2 HRMS (ASAP+, m/z): found 464.2178, calcd for C₂₆H₃₁N₃O₂FSi [M+H]+ 464.2170.

### (4-(4-(4-Fluorobenzyl)-7-((2-(trimethylsilyl)ethoxy)methyl)-7H-pyrrolo[2,3-d]pyrimidin-6-yl)phenyl)methanol (4d)

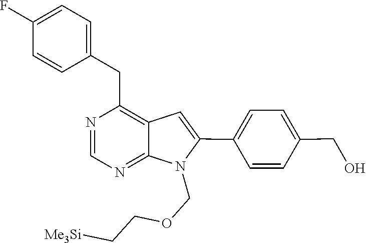 [SMILES: C[Si](C)(C)CCOCn1c(-c2ccc(CO)cc2)cc2c(Cc3ccc(F)cc3)ncnc21]

Compound 3 (298 mg, 0.741) was treated as described in General Procedure B with 4-fluorobenzylzinc bromide (3.00 mL, 2.31 mmol, 0.77 M in THF). Purification by silica-gel column chromatography (CH₂Cl₂/EtOAc—5:3, R_{f}=0.26) gave 325 mg (0.701 mmol, 82%) of a white solid, mp. 129.5-131.5° C.; ¹H NMR (600 MHz, CDCl₃) δ 8.81 (s, 1H), 7.72 (d, J=8.0 Hz, 2H), 7.49 (d, J=8.0 Hz, 2H), 7.31-7.29 (m, 2H), 6.98-6.95 (m, 2H), 6.51 (s, 2H), 5.59 (s, 2H) 4.78 (s, 2H), 4.34 (s, 2H), 3.75-3.72 (m, 2H), 0.97-0.94 (m, 2H), −0.04 (s, 9H); ¹³C NMR (150 MHz, CDCl₃) δ 161.7 (d, J=244.5 Hz), 160.1, 153.2, 151.5, 142.5, 142.2, 133.7 (d, J=3.1 Hz), 130.5 (d, J=7.8 Hz, 2C), 130.1, 129.5 (2C), 127.2 (2C), 117.4, 115.4 (d, J=21.6 Hz, 2C), 99.3, 70.7, 66.8, 64.5, 41.2, 17.9, −1.5 (3C); ¹⁹F-NMR (376 MHz, CDCl₃, C₆F₆): −119.4; HRMS (ASAP+, m/z): found 464.2172, calcd for C₂₆H₃₁N₃O₂FSi [M+H]⁺ 464.2170.

### (4-(4-(4-Methylbenzyl)-7-((2-(trimethylsilyl)ethoxy)methyl)-7H-pyrrolo[2,3-d]pyrimidin-6-yl)phenyl)methanol (4e)

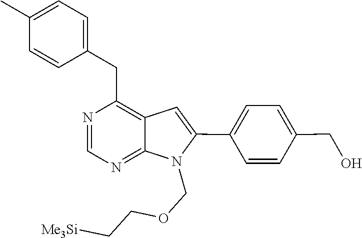 [SMILES: Cc1ccc(Cc2ncnc3c2cc(-c2ccc(CO)cc2)n3COCC[Si](C)(C)C)cc1]

Compound 3 (200 mg, 0.513 mmol) was treated as described in General Procedure B with p-methylbenzylzinc bromide (2.26 mL, 1.54 mmol, 0.68 M in THF). Quenching was done with water (20 mL cont. 6% NH₃) followed by extraction with EtOAc (3×20 mL). Purification by silica-gel column chromatography (CH₂Cl₂/EtOAc—5:2, R_{f}=0.24) gave 210 mg (0.456 mmol, 89%) of an off-white solid, mp. 85.5-86.0° C.; ¹H NMR (600 MHz, CDCl₃) δ 8.84 (s, 1H), 7.73 (d, J=8.2 Hz, 2H), 7.48 (d, J=8.2 Hz, 2H), 7.26-7.24 (m, 2H), 7.10 (d, J=7.7 Hz, 2H), 6.54 (s, 1H), 5.59 (s, 2H), 4.78 (d, J=5.3 Hz, 2H), 4.34 (s, 2H), 3.75-3.72 (m, 2H), 2.30 (s, 3H), 2.00-1.98 (m, 1H), 0.97-0.94 (m, 2H), −0.04 (s, 9H); ¹³C NMR (150 MHz, CDCl₃) δ 160.8, 153.3, 151.7, 142.0, 141.7, 136.2, 135.1, 130.5, 129.6 (2C), 129.3 (2C), 129.0 (2C), 127.2 (2C), 117.4, 99.6, 70.7, 66.8, 64.9, 41.9, 21.0, 18.0, −1.4 (3C); HRMS (ASAP+, m/z): found 460.2425, calcd for C₂₇H₃₄N₃O₂Si [M+H]+ 460.2420.

### (4-(4-(4-Isopropylbenzyl)-7-((2-(trimethylsilyl)ethoxy)methyl)-7H-pyrrolo[2,3-d]pyrimidin-6-yl)phenyl)methanol (4f)

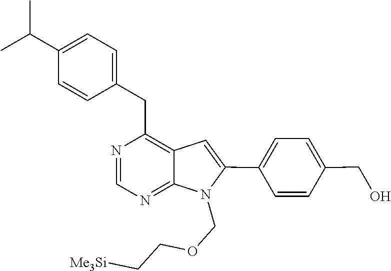 [SMILES: CC(C)c1ccc(Cc2ncnc3c2cc(-c2ccc(CO)cc2)n3COCC[Si](C)(C)C)cc1]

Compound 3 (200 mg, 0.513 mmol) was treated as described in General Procedure B with 4-isopropylbenzylzinc bromide (1.63 mL, 1.03 mmol, 0.63 M in THF). Quenching was done with water (20 mL cont. 6% NH₃) followed by extraction with EtOAc (3×20 mL). Purification by silica-gel column chromatography (n-pentane/CH₂Cl₂/1,4-dioxane—13:4:3, R_{f}=0.26) gave 82 mg (0.167 mmol, 39%) of yellow oil; ¹H NMR (600 MHz, CDCl₃) δ 8.84 (s, 1H), 7.73 (d, J=8.2 Hz, 2H), 7.48 (d, J=8.2 Hz, 2H), 7.29 (d, J=8.1 Hz, 2H), 7.16 (d, J=8.1 Hz, 2H), 6.51 (s, 1H), 5.59 (s, 2H), 4.78 (s, 2H), 4.35 (s, 2H), 3.75-3.72 (m, 2H), 2.86 (sept., J=7.0 Hz, 1H), 1.96 (s, 1H), 1.21 (d, J=7.1 Hz, 6H), 0.97-0.94 (m, 2H), −0.04 (s, 9H); ¹³C NMR (150 MHz, CDCl₃) δ 160.8, 153.3, 151.7, 147.2, 142.0, 141.7, 135.4, 130.5, 129.6 (2C), 129.0 (2C), 127.2 (2C), 126.7 (2C), 117.5, 99.6, 70.7, 66.9, 64.9, 41.9, 33.7, 24.0 (2C), 18.0, −1.4 (3C); HRMS (ASAP+, m/z): found 488.2734, calcd for C₂₉H₃₈N₃O₂Si [M+H]+ 488.2733.

### (4-(4-(4-(Tert-butyl)benzyl)-7-((2-(trimethylsilyl)ethoxy)methyl)-7H-pyrrolo[2,3-d]pyrimidin-6-yl)phenyl)methanol (4g)

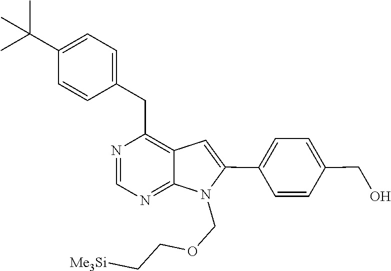 [SMILES: CC(C)(C)c1ccc(Cc2ncnc3c2cc(-c2ccc(CO)cc2)n3COCC[Si](C)(C)C)cc1]

Compound 3 (300 mg, 0.769 mmol) was treated as described in General Procedure B with tert-butylbenzylzinc bromide solution (3.34 mL, 2.31 mmol, 0.69 M in THF). The reaction time was 2.33 h. Purification by silica-gel column chromatography (CH₂Cl₂/EtOAc—5:3, R_{f}=0.28) gave 338 mg (0.673 mmol, 88%) of a white solid, mp. 52.7-54.2° C.; ¹H NMR (600 MHz, CDCl₃) δ 8.84 (s, 1H), 7.73 (d, J=7.8 Hz, 2H), 7.48 (d, J=7.8 Hz, 2H), 7.32-7.29 (m, 4H), 6.52 (s, 1H), 5.59 (s, 2H), 4.78 (d, J=4.9 Hz, 2H), 4.35 (s, 2H), 3.75-3.72 (m, 2H), 1.91 (m, 1H), 1.28 (s, 9H), 0.97-0.94 (m, 2H), −0.04 (s, 9H); ¹³C NMR (150 MHz, CDCl₃) δ 160.7, 153.3, 151.7, 149.5, 142.0, 141.7, 135.1, 130.5, 129.5 (2C), 128.8 (2C), 127.2 (2C), 125.5 (2C), 117.5, 99.6, 70.7, 66.8, 64.9, 41.7, 34.4, 31.3 (3C), 18.0, −1.4 (3C); HRMS (ASAP+, m/z): found 502.2892, calcd for C₃₀H₄₀N₃O₂Si [M+H]⁺ 502.2890.

### (4-(4-(4-(Trifluoromethyl)benzyl)-7-((2-(trimethylsilyl)ethoxy)methyl)-7H-pyrrolo[2,3-d]pyrimidin-6-yl)phenyl)methanol (4h)

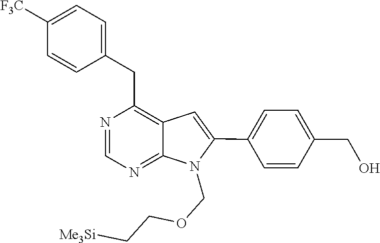 [SMILES: C[Si](C)(C)CCOCn1c(-c2ccc(CO)cc2)cc2c(Cc3ccc(C(F)(F)F)cc3)ncnc21]

Compound 3 (199 mg, 0.509 mmol) was treated as described in General Procedure B with 4-(trifluoromethyl)benzylzinc bromide (2.75 mL, 1.54 mmol, 0.56 M in THF). Quenching was done with water (20 mL cont. 6% NH₃) followed by extraction with EtOAc (3×20 mL). Purification two times with silica-gel column chromatography (CH₂Cl₂/EtOAc—5:5, R_{f}=0.26, then n-pentane/1,4-dioxane—92.5:7.5, R_{f}=0.21) gave 101 mg (0.197 mmol, 39%) of an colorless oil; ¹H NMR (600 MHz, CDCl₃) δ 8.86 (s, 1H), 7.74 (d, J=8.0 Hz, 2H), 7.55 (d, J=8.2 Hz, 2H), 7.50-7.46 (m, 4H), 6.55 (s, 1H), 5.60 (s, 2H), 4.79 (s, 2H), 4.44 (s, 2H), 3.75-3.73 (m, 2H), 2.13 (s, 1H), 0.97-0.94 (m, 2H), −0.04 (s, 9H); ¹³C NMR (150 MHz, CDCl₃) δ 159.1, 153.3, 151.6, 142.7, 142.1, 142.0, 130.2, 129.6 (2C), 129.4 (2C), 129.1 (q, J=32.2 Hz), 127.3 (2C), 125.6 (q, J=3.4 Hz, 2C), 124.1 (q, J=271.6 Hz), 117.5, 99.1, 70.8, 67.0, 64.8, 41.7, 18.0, −1.4 (3C); ¹⁹F-NMR (376 MHz, CDCl₃, C₆F₆): −65.6; HRMS (ASAP+, m/z): found 514.2138, calcd for C₂₇H₃₁N₃O₂F₃Si [M+H]⁺ 514.2128.

### (4-(4-(1-phenylethyl)-7-((2-(trimethylsilyl)ethoxy)methyl)-7H-pyrrolo[2,3-d]pyrimidin-6-yl)phenyl)methanol (4i)

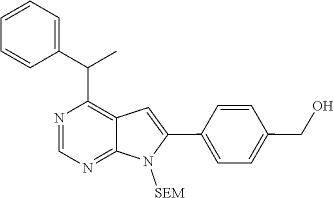 [SMILES: CC(c1ccccc1)c1ncnc2c1cc(-c1ccc(CO)cc1)n2COCC[Si](C)(C)C]

Compound 3 (400 mg, 1.03 mmol) was treated as described in General Procedure B with α-methylbenzylzinc bromide (6.18 mL, 3.09 mmol, 0.5 M in THF). The reaction time was 2.33 h. Purification by silica-gel column chromatography (CH₂Cl₂/EtOAc—5:1, R_{f}=0.19) gave 439 mg (0.896 mmol, 93%) of a pale yellow solid, mp. 88-89° C.; ¹H NMR (600 MHz, CDCl3) δ 8.90 (s, 1H), 7.70 (d, J=8.1 Hz, 2H), 7.47 (d, J=8.1 Hz, 2H), 7.41 (d, J=7.5 Hz, 2H), 7.29 (t, J=7.5 Hz, 2H), 7.20-7.18 (m, 1H), 6.46 (s, 1H), 5.57 (s, 2H), 4.77 (d, J=5.5 Hz, 2H), 4.62 (q, J=7.1 Hz, 1H), 3.75-3.71 (m, 2H), 1.93 (t, J=5.8 Hz, 1H), 1.84 (d, J=7.1 Hz, 3H), 0.97-0.93 (m, 2H), −0.04 (s, 9H); ¹³C NMR (150 MHz, CDCl₃) δ 164.3, 153.3, 151.6, 143.9, 141.8, 141.6, 130.5, 129.6 (2C), 128.5 (2C), 127.8 (2C), 127.2 (2C), 126.6, 116.8, 99.6, 70.6, 66.8, 64.9, 45.0, 19.8, 18.0, −1.4 (3C); HRMS (ASAP+, m/z): found 460.2419, calcd for C₂₇H₃₄N₃O₂Si [M+H]⁺ 460.2420.

### 4-Benzyl-6-(4-(((tert-butyldimethylsilyl)oxy)methyl)phenyl)-7-((2-(trimethylsilyl)ethoxy)methyl)-7H-pyrrolo[2,3-d]pyrimidine (6a)

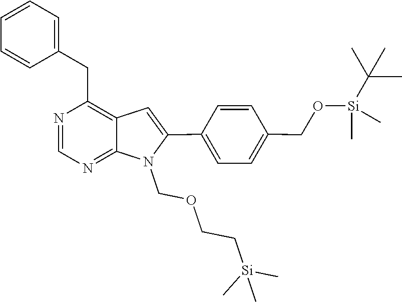 [SMILES: CC(C)(C)[Si](C)(C)OCc1ccc(-c2cc3c(Cc4ccccc4)ncnc3n2COCC[Si](C)(C)C)cc1]

The corresponding 4-chloropyrrolopyrimidine (379 mg, 0.751 mmol) was treated as described in General Procedure B with benzylzinc bromide (2.20 mmol in 2.25 mL THF) and Pd(PPh₃)₄ (21.6 mg, 0.0187 mmol). The reaction time was 1 h. The crude reaction mixture was extracted from an aqueous ammonia solution (5% by vol.) using EtOAc. The organic phase was washed using aqueous ammonia (10% by vol.) followed by brine. Purification by silica-gel column chromatography (n-pentane/EtOAc—5:1, R_{f}=0.19) gave 414 mg (0.739 mmol, 98%) of a colorless solid; ¹H NMR (400 MHz, CDCl₃) δ 8.85 (s, 1H), 7.71-7.67 (m, 2H), 7.46-7.41 (m, 2H), 7.38-7.34 (m, 2H), 7.31-7.26 (m, 2H), 7.24-7.19 (m, 1H), 6.50 (s, 1H), 5.59 (s, 2H), 4.81 (s, 2H), 4.39 (s, 2H), 3.76-3.69 (m, 2H), 0.97 (s, 9H), 0.13 (s, 6H), −0.04 (s, 9H); HRMS (ES+, m/z): found 560.3133, calcd for C₃₂H₄₆N₃O₂Si₂ [M+H]⁺ 560.3129.

### 6-(4-(((Tert-butyldimethylsilyl)oxy)methyl)phenyl)-4-(1-phenylethyl)-7-((2-(trimethylsilyl)ethoxy)methyl)-7H-pyrrolo[2,3-d]pyrimidine (6i)

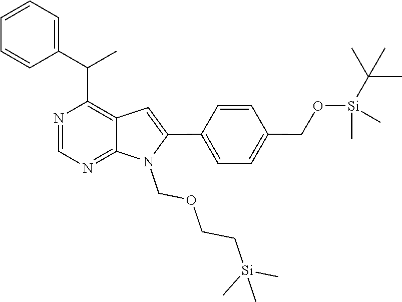 [SMILES: CC(c1ccccc1)c1ncnc2c1cc(-c1ccc(CO[Si](C)(C)C(C)(C)C)cc1)n2COCC[Si](C)(C)C]

Compound 6a (30 mg, 0.053 mmol) was dissolved in THF (1.0 mL) and cooled on an ice-bath. To the solution, lithium hexamethyldisilazide (65 μL, 0.065 mmol, 1.0 M in THF) was added dropwise over 2 min. After stirring for 10 min, methyl iodide (53 μL, 0.106 mmol, 2 M in TBME) was added. The mixture was stirred for a further 10 min before quenching the reaction by adding one drop of brine solution (5 M NaCl_{(aq)}). The volatiles were removed in vacuo, and the residue partitioned between EtOAc (10 mL) and water (10 mL). The aqueous phase was extracted using EtOAc (2×5 mL) and the combined organic phases were washed with water (3×5 mL) and brine (10 mL), dried over anhydrous Na₂SO₄, filtered and concentrated in vacuo. The crude product was purified by silica-gel column chromatography (CH₂Cl₂/n-pentane/TBME—75:25:5) giving 67 mg (0.116 mmol, 81%) of an off-white solid. ¹H NMR (600 MHz, CDCl₃) δ 8.90 (s, 1H), 7.65-7.62 (m, 2H), 7.44-7.39 (m, 4H), 7.30-7.27 (m, 2H), 7.21-7.18 (m, 1H), 6.45 (s, 1H), 5.59 (s, 2H), 4.80 (s, 2H), 4.62 (q, J=7.1 Hz, 1H), 3.75-3.68 (m, 2H), 1.83 (d, J=7.2 Hz, 3H), 0.97 (s, 11H), 0.13 (s, 6H), −0.05 (s, 9H);

### 6-(4-(((Tert-butyldimethylsilyl)oxy)methyl)phenyl)-4-(1-phenylpropyl)-7-((2-(trimethylsilyl)ethoxy)methyl)-7H-pyrrolo[2,3-d]pyrimidine (6j)

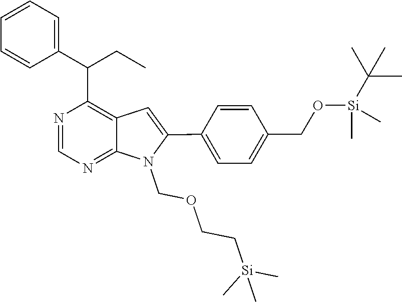 [SMILES: CCC(c1ccccc1)c1ncnc2c1cc(-c1ccc(CO[Si](C)(C)C(C)(C)C)cc1)n2COCC[Si](C)(C)C]

Compound 6a (60 mg, 0.108 mmol) was dissolved in THF (2.0 mL) and cooled on an ice-bath. To the solution, lithium hexamethyldisilazide (130 μL, 0.130 mmol, 1.0 M in THF) was added dropwise over 2 min. After stirring for 10 min, ethyl iodide (20 μL, 0.249 mmol) was added. The mixture was stirred for a further 25 min before quenching the reaction by adding two drops of brine solution (5 M NaCl_{(aq)}). The volatiles were removed in vacuo, and the residue partitioned between EtOAc (10 mL) and water (10 mL). The aqueous phase was extracted using EtOAc (2×5 mL) and the combined organic phases were washed with water (3×5 mL) and brine (10 mL), dried over anhydrous Na₂SO₄, filtered and concentrated in vacuo. The crude product was purified by silica-gel column chromatography (CH₂Cl₂/n-pentane/TBME—75:25:5, R_{f}=0.22) giving 54 mg (0.092 mmol, 85%) of an off-white solid. ¹H NMR (600 MHz, CDCl₃) δ 8.90 (s, 1H), 7.72-7.67 (m, 2H), 7.47-7.41 (m, 4H), 7.29-7.26 (m, 2H), 7.21-7.15 (m, 1H), 6.60 (s, 1H), 5.57 (s, 2H), 4.81 (s, 2H), 4.30 (t, J=7.6 Hz, 1H), 3.75-3.69 (m, 2H), 2.53-2.43 (m, 1H), 2.28-2.19 (m, 1H), 0.97 (s, 9H), 0.94 (s, 5H), 0.13 (s, 6H), −0.05 (s, 9H); ¹³C NMR (151 MHz, CDCl₃) δ 163.4, 153.2, 151.6, 142.7, 142.4, 142.2, 129.8, 129.3 (2C), 128.5 (2C), 128.2 (2C), 126.6, 126.3 (2C), 117.6, 99.2, 70.6, 66.8, 64.6, 52.7, 27.4, 26.0 (3C), 18.4, 18.0, 12.7, −1.4 (3C), −5.2 (2C); HRMS (ASAP+, m/z): found 588.3433, calcd for C₃₄H₅₀N₃O₂Si₂ [M+H]⁺ 588.3442.

### 6-(4-(((Tert-butyldimethylsilyl)oxy)methyl)phenyl)-4-(fluoro(phenyl)methyl)-7-((2-(trimethylsilyl)ethoxy)methyl)-7H-pyrrolo[2,3-d]pyrimidine (6k)

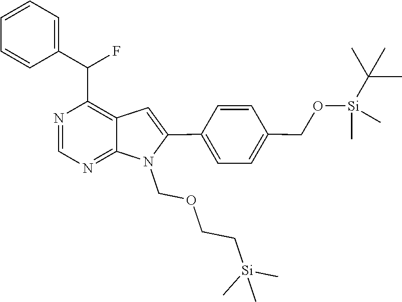 [SMILES: CC(C)(C)[Si](C)(C)OCc1ccc(-c2cc3c(C(F)c4ccccc4)ncnc3n2COCC[Si](C)(C)C)cc1]

Compound 6a (81 mg, 0.144 mmol) was dissolved in THF (1.5 mL) and cooled on an ice-bath. To the solution, lithium hexamethyldisilazide (175 μL, 1.0 M in THF) was added dropwise over 3 min. After stirring for 10 min, N-Fluorobenzenesulfonimide (68 mg, 0.216 mmol) dissolved in THF (0.5 mL) was added. The mixture was stirred fora further 10 min before quenching the reaction by adding one drop of brine solution (5 M NaCl_{(aq)}). The volatiles were removed in vacuo, and the residue partitioned between EtOAc (10 mL) and water (10 mL). The aqueous phase was extracted using EtOAc (2×5 mL) and the combined organic phases were washed with water (3×5 mL) and brine (10 mL), dried over anhydrous Na₂SO₄, filtered and concentrated in vacuo. The crude product was purified by silica-gel column chromatography (CH₂Cl₂/n-pentane/TBME—15:5:1) giving 67 mg (0.116 mmol, 81%) of an off-white solid, mp. 96-98° C. TLC (silica, CH₂Cl₂/n-pentane/TBME—75:25:10): R_{f}=0.48; ¹H NMR (600 MHz, CDCl₃) δ 8.84 (d, J=1.1 Hz, 1H), 7.77-7.73 (m, 2H), 7.56-7.51 (m, 2H), 7.48-7.43 (m, 2H), 7.40-7.32 (m, 3H), 6.88 (d, J=3.2 Hz, 1H), 6.73 (d, ²J_{HF}=46.7 Hz, 1H), 5.67-5.59 (m, 2H), 4.82 (s, 2H), 3.77-3.71 (m, 2H), 0.97 (s, 9H), 0.98-0.95 (m, 2H), 0.14 (s, 6H), −0.04 (s, 9H); ¹³C NMR (151 MHz, CDCl₃) δ 158.2 (d, J_{CF}=29.2 Hz, 1C), 154.3, 151.0 (d, J_{CF}=2.2 Hz), 143.5 (d, J_{CF}=2.4 Hz, 1C), 142.7, 137.9 (d, J_{CF}=21.0 Hz, 1C), 129.5, 129.4 (2C), 129.0 (d, J_{CF}=2.3 Hz, 1C), 128.7 (2C), 126.7 (d, J=6.3 Hz, 2C), 126.3 (2C), 115.1, 100.2 (d, J=6.9 Hz, 1C), 95.4 (d, J=174.8 Hz, 1C), 70.7, 66.9, 64.6, 26.0 (3C), 18.4, 18.0, −1.4 (3C), —5.2 (2C); ¹⁹F NMR (376 MHz, CDCl₃) δ −178.2 (d, J=46.7 Hz); HRMS (ASAP+, m/z): found 578.3029, calcd for C₃₂H₄₅N₃O₂FSi₂ [M+H]⁺ 578.3034.

### Synthesis of 6-iodo-4-phenoxy-7-((2-(trimethylsilyl)ethoxy)-methyl)-7H-pyrrolo[2,3-d]pyrimidine (7)

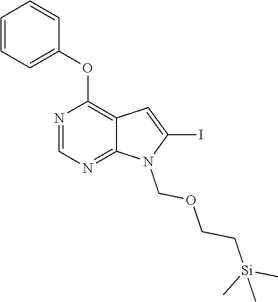 [SMILES: C[Si](C)(C)CCOCn1c(I)cc2c(Oc3ccccc3)ncnc21]

In a closed reaction vessel under an N₂ atmosphere 4-chloro-6-iodo-7-((2-(trimethylsilyl)ethoxy)methyl)-7Hpyrrolo[2,3-d]pyrimidine (1.51 g, 3.69 mmol), Cs₂CO₃ (2.99 g, 9.18 mmol) and phenol (414 mg, 4.39 mmol) were dissolved in dry DMF (20 mL) and heated to 70° C. After 24 h, the reaction was cooled down to 22° C. and diluted with EtOAc (40 mL). The organic phase was washed with aq. saturated NaHCO₃ solution (20 mL), water (2×20 mL) and with brine (30 mL). The organic fraction was dried over Na₂SO₄ and the solvent evaporated under reduced pressure. The residue was absorbed onto Celite and purified using silica-gel flash chromatography (n-pentane/EtOAc—10:1, R_{f}=0.26). This gave 1.54 g (3.29 mmol, 85%) of 6-iodo-4-phenoxy-7-((2-(trimethylsilyl)ethoxy)methyl)-7H-pyrrolo[2,3-d]pyrimidine as a colourless solid, mp. 100-102.5° C.; ¹H NMR (600 MHz, CDCl₃) δ: 8.39 (s, 1H), 7.47-7.44 (m, 2H), 7.31-7.28 (m, 1H), 7.24-7.22 (m, 2H), 6.85 (s, 1H), 5.67 (s, 2H), 3.60 (t, J=8.2 Hz, 2H), 0.94 (t, J=8.3 Hz, 2H), −0.04 (s, 9H); ¹³C NMR (150 Hz, CDCl₃) δ: 160.9, 154.9, 152.8, 151.5, 129.9 (2C), 125.9, 121.9 (2C), 110.5, 107.9, 82.0, 73.6, 66.7, 17.9, −1.3 (3C); HRMS (ASAP+, m/z): found 468.0611, calcd for C₁₈H₂₃IN₃O₂Si [M+H]+ 468.0604.

### (4-(4-Phenoxy-7-((2-(trimethylsilyl)ethoxy)methyl)-7H-pyrrolo[2,3-d]pyrimidin-6-yl)phenyl)methanol (8)

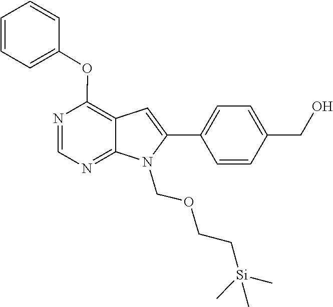 [SMILES: C[Si](C)(C)CCOCn1c(-c2ccc(CO)cc2)cc2c(Oc3ccccc3)ncnc21]

6-Iodo-4-phenoxy-7-((2-(trimethylsilyl)ethoxy)methyl)-7Hpyrrolo[2,3-d]pyrimidine (1.00 g, 2.14 mmol), (4-(hydroxymethyl)-phenyl)boronic acid (394 mg, 2.59 mmol), K2CO3 (1.04 g, 7.49 mmol) and XPhos 2nd generation precatalyst (86 mg, 0.11 mmol) were added in a Schlenk tube under nitrogen atmosphere and diluted in degassed water and 1,4-dioxane (12 mL, v/v, 1/1) and slowly heated to 100° C. According to TLC, full conversion was reached after 2 h. The reaction mixture was cooled to 22° after 3 h and diluted with water (50 mL) and EtOAc (90 mL). After phase separation, the water phase was extracted with more EtOAc (4×50 mL). The combined organic phase was washed with brine (100 mL) and dried over Na2SO4. The crude product was purified first using silica-gel flash chromatography (DCM/EtOAc, 10:1, R_{f}=0.17) and then silica-gel flash chromatography (n-pentane/EtOAc, 1:5, Rf=0.70) giving 695 mg (1.55 mmol, 73%) of (4-(4-phenoxy-7-((2-(trimethylsilyl)ethoxy)methyl)-7Hpyrrolo[2,3-d]pyrimidin-6-yl)phenyl)methanol as a highly viscous yellow-orange oil. ¹H NMR (600 MHz, DMSO-d₆) δ: 8.42 (s, 1H), 7.73 (d, J=8.3 Hz, 2H), 7.49-7.46 (m, 4H), 7.32-7.28 (m, 3H), 6.72 (s, 1H), 5.61 (s, 2H), 5.30 (t, J=5.7 Hz, 1H), 4.57 (d, J=5.7 Hz, 2H), 3.63 (t, J=8.0 Hz, 2H), 0.86 (t, J=8.0 Hz, 2H), −0.08 (s, 9H); ¹³C NMR (150 Hz, DMSO-d₆) δ: 161.4, 154.7, 152.6, 150.6, 143.5, 140.7, 129.7 (2C), 129.0, 128.7 (2C), 126.8 (2C), 125.5, 121.9 (2C), 105.0, 98.1, 70.8, 66.0, 62.5, 17.3, −1.4 (3C); HRMS (ES+, m/z): found 448.2059, calcd for C₂₅H₃₀N₃O₃Si [M+H]⁺ 448.2056.

### Inhibitor Structures

### General Procedure C: Deprotection of the SEM Group

The SEM-protected pyrrolopyrimidine (0.2-0.6 mmol) was stirred in trifluoroacetic acid (0.5 mL) and CH₂Cl₂ (3 mL) at 50° C. for 3 h. The reaction mixture was concentrated in vacuo before it was stirred in MeOH (10 mL) and aqueous ammonia (20 mL, 25%) for 2-3 h. In some cases, THF (1 mL) was added to increase solubility. The reaction mixture was again concentrated in vacuo, and the crude product was purified by silica-gel column chromatography as specified for each compound.

### (4-(4-Benzyl-7H-pyrrolo[2,3-d]pyrimidin-6-yl)phenyl)methanol (5a)

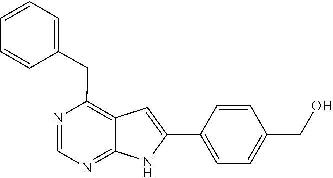 [SMILES: OCc1ccc(-c2cc3c(Cc4ccccc4)ncnc3[nH]2)cc1]

The SEM intermediate 4a (38 mg, 0.084 mmol) was treated as described in General Procedure C. Purification by silica-gel column chromatography (CH₂Cl₂/MeOH—92.5:7.5, R_{f}=0.22) gave 24 mg (0.075 mmol, 89%) of a colorless powder, mp.>250° C. (decomp.). HPLC purity: 99%, t_{R}=7.8 min^{†}; ¹H NMR (400 MHz, DMSO-d₆) δ 12.51 (s, 1H) 8.64 (s, 1H), 7.96-7.87 (m, 2H), 7.46-7.37 (m, 4H), 7.33-7.24 (m, 2H), 7.24-7.15 (m, 2H), 5.26 (t, J=5.8 Hz, 1H), 4.54 (d, J=5.6 Hz, 2H), 4.32 (s, 2H); ¹³C NMR (100 MHz, DMSO-d₆) δ 160.2, 153.1, 151.5, 143.6, 139.1, 139.0, 129.7, 129.5 (2C), 128.9 (2C), 127.4 (2C), 126.8, 125.8 (2C), 117.5, 96.2, 63.0, 41.6; HRMS (ASAP+, m/z): found 316.1454, calcd for C₂₀H₁₈N₃O [M+H]⁺ 316.1450.

### (4-(4-(2-Fluorobenzyl)-7H-pyrrolo[2,3-d]pyrimidin-6-yl)phenyl)methanol (5b)

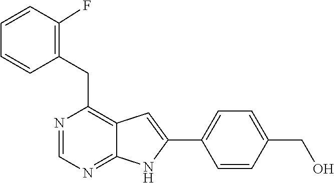 [SMILES: OCc1ccc(-c2cc3c(Cc4ccccc4F)ncnc3[nH]2)cc1]

The SEM intermediate 4a (130 mg, 0.280 mmol) was treated as described in General Procedure C, but with addition of THF (1 mL) in the last hydrolysis step. Purification by silica-gel column chromatography (CH₂Cl₂/MeOH—95:5, R_{f}=0.11) gave 83 mg (0.248 mmol, 88%) of a white solid, mp.>227° C. (decomp.); HPLC purity: 99%; ¹H NMR (600 MHz, DMSO-d₆) δ 12.54 (s, 1H), 8.61 (s, 1H), 7.89 (d, J=8.2 Hz, 2H), 7.43 (d, J=8.2 Hz, 2H), 7.40 (td, J=7.7 and 1.6 Hz, 1H), 7.31-7.27 (m, 1H), 7.18-7.13 (m, 2H), 7.04 (s, 1H), 5.26 (t, J=5.7 Hz, 1H), 4.55 (d, J=5.6 Hz, 2H), 4.38 (s, 2H); ¹³C NMR (150 MHz, DMSO-d₆) δ 160.5 (d, J=244.9 Hz), 158.5, 152.6, 151.0, 143.1, 138.6, 131.8 (d, J=4.5 Hz), 129.2, 128.6 (d, J=8.0 Hz), 127.0 (2C), 125.4 (2C), 125.3 (d, J=16.6 Hz), 124.4 (d, J=3.3 Hz, 1C), 117.9, 115.2 (d, J=21.7 Hz), 95.4, 62.5, 34.0 (d, J=1.2 Hz); ¹⁹F-NMR (376 MHz, DMSO-d₆, C₆F₆): δ −119.2; HRMS (ASAP+, m/z): found 334.1357, calcd for C₂₀H₁₇N₃OF [M+H]+ 334.1356.

### (4-(4-(3-Fluorobenzyl)-7H-pyrrolo[2,3-d]pyrimidin-6-yl)phenyl)methanol (5c)

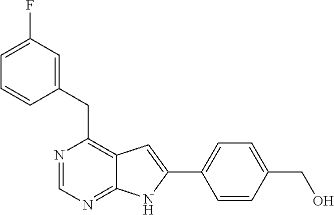 [SMILES: OCc1ccc(-c2cc3c(Cc4cccc(F)c4)ncnc3[nH]2)cc1]

The SEM intermediate 4c (76 mg, 0.164 mmol) was treated as described in General Procedure C, but with addition of THF (1 mL) in the last hydrolysis step. Purification by silica-gel column chromatography (CH₂Cl₂/MeOH—92.5:7.5, R_{f}=0.18) gave 55 mg (0.164 mmol, 51%) of a white solid, mp.>276° C. (decomp.). HPLC purity: 99%; ¹H NMR (600 MHz, DMSO-d₆) δ 12.54 (s, 1H), 8.65 (s, 1H), 7.91 (d, J=8.2 Hz, 2H), 7.43 (d, J=8.2 Hz, 2H), 7.34-7.31 (m, 1H), 7.25-7.22 (m, 3H), 7.04-7.01 (m, 1H), 5.26 (t, J=5.7 Hz, 1H), 4.55 (d, J=5.8 Hz, 2H), 4.35 (s, 2H); ¹³C NMR (150 MHz, DMSO-d₆) δ 162.1 (d, J=243.4 Hz), 159.0, 152.7, 151.0, 143.1, 141.4 (d, J=7.7 Hz), 138.7, 130.2 (d, J=8.3 Hz), 129.2, 126.9 (2C), 125.4 (2C), 125.2 (d, J=2.7 Hz), 118.0, 115.7 (d, J=21.2 Hz), 113.1 (d, J=20.8 Hz), 95.7, 62.5, 40.5; ¹⁹F-NMR (376 MHz, DMSO-d₆, C₆F₆): −115.8; HRMS (ASAP+, m/z): found 334.1362, calcd for C₂₀H₁₇N₃OF [M+H]⁺ 334.1356.

### (4-(4-(4-Fluorobenzyl)-7H-pyrrolo[2,3-d]pyrimidin-6-yl)phenyl)methanol (5d)

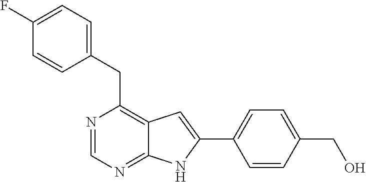 [SMILES: OCc1ccc(-c2cc3c(Cc4ccc(F)cc4)ncnc3[nH]2)cc1]

The SEM intermediate 4d (232 mg, 0.500 mmol) was treated as described in General Procedure C. Purification by silica-gel column chromatography (CH₂Cl₂/MeOH 95:5, R_{f}=0.34) gave 120 mg (0.360 mmol, 72%) as a white solid, mp.>264° C. (decomp.); HPLC purity: 98%; ¹H NMR (600 MHz, DMSO-d₆) δ 12.52 (s, 1H), 8.64 (s, 1H), 7.90 (d, J=8.2 Hz, 2H), 7.45-7.42 (m, 4H), 7.19 (s, 1H), 7.12-7.09 (m, 2H), 5.25 (t, J=5.7 Hz, 1H), 4.54 (d, J=5.7 Hz, 2H), 4.31 (s, 2H); ¹³C NMR (150 MHz, DMSO-d₆) δ 160.9 (d, J=241.2 Hz), 159.5, 152.7, 151.0, 143.1, 138.6, 134.8 (d, J=2.6 Hz), 130.8 (d, J=7.6 Hz, 2C), 129.2, 126.9 (2C), 125.3 (2C), 117.9, 115.1 (d, J=20.8 Hz, 2C), 95.7, 62.5, 40.0; ¹⁹F-NMR (376 MHz, DMSO-d₆, C₆F₆): −119.2; HRMS (ASAP+, m/z): found 334.1356, calcd for C₂₀H₁₇N₃OF [M+H]+ 334.1356.

### (4-(4-(4-Methylbenzyl)-7H-pyrrolo[2,3-d]pyrimidin-6-yl)phenyl)methanol (5e)

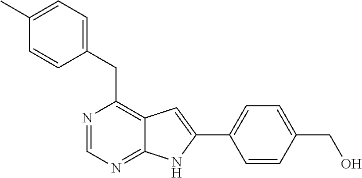 [SMILES: Cc1ccc(Cc2ncnc3[nH]c(-c4ccc(CO)cc4)cc23)cc1]

The SEM intermediate 4e (144 mg, 0.313 mmol) was treated as described in General Procedure C, but with addition of THF (1 mL) in the last hydrolysis step. Purification by silica-gel column chromatography (CH₂Cl₂/MeOH 92.5:7.5, R_{f}=0.26) gave 80 mg (0.242 mmol, 77%) as a white solid, mp.>256° C. (decomp.); HPLC purity: 97%; ¹H NMR (600 MHz, DMSO-d₆) δ 12.49 (s, 1H), 8.63 (s, 1H), 7.90 (d, J=7.9 Hz, 2H), 7.42 (d, J=8.0 Hz, 2H), 7.28 (d, J=7.7 Hz, 2H), 7.15 (s, 1H), 7.08 (d, J=7.7 Hz, 2H), 5.25 (t, J=5.7 Hz, 1H), 4.54 (d, J=5.7 Hz, 2H), 4.26 (s, 2H), 2.23 (s, 3H); ¹³C NMR (150 MHz, DMSO-d₆) δ 159.9, 152.6, 151.0, 143.0, 138.4, 135.6, 135.3, 129.3, 128.91 (2C), 128.87 (2C), 126.9 (2C), 125.3 (2C), 117.9, 95.7, 62.5, 40.7, 20.6; HRMS (ASAP+, m/z): found 330.1609, calcd for C₂₁H₂₀N₃O [M+H]⁺ 330.1606.

### (4-(4-(4-Isopropylbenzyl)-7H-pyrrolo[2,3-d]pyrimidin-6-yl)phenyl)methanol (5f)

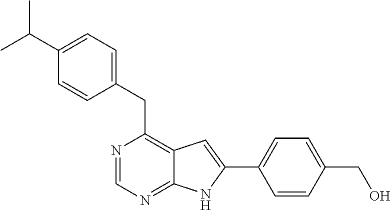 [SMILES: CC(C)c1ccc(Cc2ncnc3[nH]c(-c4ccc(CO)cc4)cc23)cc1]

The SEM intermediate 4f (68 mg, 0.139 mmol) was treated as described in General Procedure C, but with THF (10 mL) replacing MeOH in the last hydrolysis step. Purification by silica-gel column chromatography (CH₂Cl₂/MeOH 92.5:7.5, R_{f}=0.15) gave 40 mg (0.112 mmol, 81%) as a white solid, mp.>249° C. (decomp.); HPLC purity: 99%; ¹H NMR (600 MHz, DMSO-d₆) δ 12.50 (s, 1H), 8.63 (s, 1H), 7.90 (d, J=8.2 Hz, 2H), 7.42 (d, J=8.3 Hz, 2H), 7.32 (d, J=8.1 Hz, 2H), 7.15-7.14 (m, 3H), 5.26 (t, J=5.8 Hz, 1H), 4.54 (d, J=5.8 Hz, 2H), 4.27 (s, 2H), 2.81 (sept., J=6.9 Hz, 1H), 1.14 (d, J=6.9 Hz, 6H); ¹³C NMR (150 MHz, DMSO-d₆) δ 159.9, 152.6, 151.0, 146.3, 143.0, 138.4, 135.9, 129.3, 128.9 (2C), 126.9 (2C), 126.3 (2C), 125.3 (2C), 117.9, 95.8, 62.5, 40.7, 33.0, 23.9 (2C); HRMS (ASAP+, m/z): found 358.1924, calcd for C₂₃H₂₄N₃O [M+H]⁺ 358.1919.

### (4-(4-(4-(tert-Butyl)benzyl)-7H-pyrrolo[2,3-d]pyrimidin-6-yl)phenyl)methanol (5g)

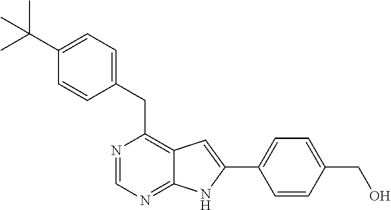 [SMILES: CC(C)(C)c1ccc(Cc2ncnc3[nH]c(-c4ccc(CO)cc4)cc23)cc1]

The SEM intermediate 4g (302 mg, 0.602 mmol) was treated as described in General Procedure C. Purification by silica-gel column chromatography (CH₂Cl₂/MeOH—95:5, R_{f}=0.26) gave 199 mg (0.536 mmol, 89%) as a white solid, mp.>263° C. (decomp.); HPLC purity: >98%; ¹H NMR (600 MHz, DMSO-d₆) δ 12.49 (s, 1H), 8.63 (s, 1H), 7.90 (d, J=8.1 Hz, 2H), 7.42 (d, J=8.1 Hz, 2H), 7.33-7.32 (m, 2H), 7.30-7.28 (m, 2H), 7.14 (s, 1H), 5.25 (t, J=5.7 Hz, 1H), 4.54 (d, J=5.8 Hz, 2H), 4.27 (s, 2H), 1.22 (s, 9H). ¹³C NMR (150 MHz, DMSO-d₆) δ 159.9, 152.6, 151.0, 148.6, 143.0, 138.4, 135.5, 129.2, 128.6 (2C), 126.9 (2C), 125.3 (2C), 125.1 (2C), 117.8, 95.7, 62.5, 40.6, 34.0, 31.1 (3C); HRMS (ASAP+, m/z): found 372.2076, calcd for C₂₄H₂₆N₃O [M+H]⁺ 372.2076.

### (4-(4-(4-(Trifluoromethyl)benzyl)-7H-pyrrolo[2,3-d]pyrimidin-6-yl)phenyl)methanol (4h)

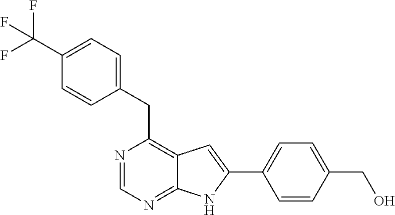 [SMILES: OCc1ccc(-c2cc3c(Cc4ccc(C(F)(F)F)cc4)ncnc3[nH]2)cc1]

The SEM intermediate 4h (75 mg, 0.150 mmol) was treated as described in General Procedure C, but with addition of THF (1 mL) in the last hydrolysis step. Purification by silica-gel column chromatography (CH₂Cl₂/MeOH 92.5:7.5, R_{f}=0.20) gave 47 mg (0.124 mmol, 85%) as a white solid, mp.>273° C. (decomp.); HPLC purity: 96%; ¹H NMR (600 MHz, DMSO-d₆) δ 12.57 (s, 1H), 8.65 (s, 1H), 7.91 (d, J=8.2 Hz, 2H), 7.67-7.61 (m, 4H), 7.43 (d, J=8.3 Hz, 2H), 7.23 (s, 1H), 5.27 (t, J=5.7 Hz, 1H), 4.55 (d, J=5.7 Hz, 2H), 4.44 (s, 2H); ¹³C NMR (150 MHz, DMSO-d₆) δ 158.7, 152.7, 151.1, 143.5, 143.2, 138.8, 129.9 (2C), 129.2, 127.0 (q, J=32.4 Hz), 126.9 (2C), 125.4 (2C), 125.2 (q, J=3.4 Hz, 2C), 124.6 (q, J=271.0 Hz), 118.1, 95.6, 62.5, 40.5; ¹⁹F-NMR (400 MHz, DMSO-d₆, C₆F₆): −63.2; HRMS (ASAP+, m/z): found 384.1327, calcd for C₂₁H₁₇N₃OF₃ [M+H]⁺ 384.1324.

### (4-(4-(1-Phenylethyl)-7H-pyrrolo[2,3-d]pyrimidin-6-yl)phenyl)methanol (5i)

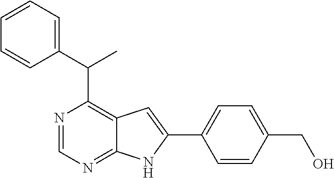 [SMILES: CC(c1ccccc1)c1ncnc2[nH]c(-c3ccc(CO)cc3)cc12]

The SEM intermediate 4i (195 mg, 0.424 mmol) was treated as described in General Procedure C. Purification by silica-gel column chromatography (CH₂Cl₂/MeOH—95:5, Rf=0.28) gave 115 mg (0.349 mmol, 82%) as a white solid, mp.>229° C. (decomp.); HPLC purity: >99%; ¹H NMR (600 MHz, DMSO-d₆) δ 12.48 (s, 1H), 8.70 (s, 1H), 7.88 (d, J=8.1 Hz, 2H), 7.47 (d, J=7.5 Hz, 2H), 7.41 (d, J=8.1 Hz, 2H), 7.27 (t, J=7.7 Hz, 2H), 7.18-7.15 (m, 1H), 7.12 (s, 1H), 5.24 (t, J=5.8 Hz, 1H), 4.69 (q, J=7.1 Hz, 1H), 4.54 (d, J=5.8 Hz, 2H), 1.72 (d, J=7.1 Hz, 3H); ¹³C NMR (150 MHz, DMSO-d₆) δ 163.1, 152.6, 151.0, 144.2, 143.0, 138.4, 129.2, 128.3 (2C), 127.6 (2C), 126.9 (2C), 126.3, 125.3 (2C), 117.3, 95.6, 62.5, 43.5, 19.7; HRMS (ASAP+, m/z): found 330.1606, calcd for C₂₁H₂₀N₃O [M+H]+ 330.1606.

### (4-(4-(1-Phenylpropyl)-7H-pyrrolo[2,3-d]pyrimidin-6-yl)phenyl)methanol (5j)

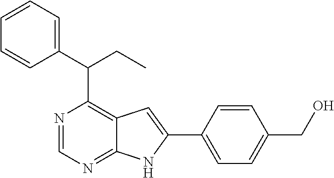 [SMILES: CCC(c1ccccc1)c1ncnc2[nH]c(-c3ccc(CO)cc3)cc12]

The SEM/TBDMS intermediate 6j (46 mg, 0.078 mmol) was treated as described in General Procedure C. Purification by silica-gel column chromatography (CH₂Cl₂/MeOH—93.5:6.5, R_{f}=0.23) gave 25 mg (0.073 mmol, 94%) of a colorless solid, mp.>250° C. (decomp.). ¹H NMR (600 MHz, DMSO-d₆) δ 12.47 (s, 1H), 8.71 (s, 1H), 7.92-7.88 (m, 2H), 7.54-7.49 (m, 2H), 7.43-7.40 (m, 2H), 7.28-7.25 (m, 2H), 7.23 (s, 1H), 7.18-7.12 (m, 1H), 5.27 (t, J=5.7 Hz, 1H), 4.54 (d, J=5.7 Hz, 2H), 4.41 (t, J=7.6 Hz, 1H), 2.42-2.33 (m, 1H), 2.16-2.06 (m, 1H), 0.85 (t, J=7.3 Hz, 3H); ¹³C NMR (151 MHz, DMSO-d₆) δ 162.4, 152.6, 151.1, 143.1, 143.0, 138.5, 129.3, 128.3 (2C), 128.1 (2C), 127.0 (2C), 126.4, 125.4 (2C), 118.1, 95.7, 62.6, 51.3, 26.9, 12.6; HRMS (ES+, m/z): found 344.1768, calcd for C₂₂H₂₂N₃O [M+H]⁺ 344.1763.

### (4-(4-(Fluoro(phenyl)methyl)-7H-pyrrolo[2,3-d]pyrimidin-6-yl)phenyl)methanol (5k)

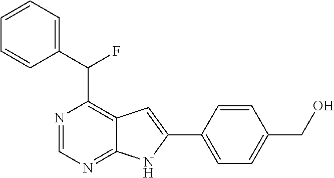 [SMILES: OCc1ccc(-c2cc3c(C(F)c4ccccc4)ncnc3[nH]2)cc1]

The SEM/TBDMS intermediate 6k (59 mg, 0.102 mmol) was treated as described in General Procedure C. Purification by silica-gel column chromatography (CH₂Cl₂/MeOH—93.5:6.5, R_{f}=0.19) gave 31 mg (0.092 mmol, 90%) of a colorless solid, mp.>250° C. (decomp.). ¹H NMR (600 MHz, DMSO-d₆) δ 12.73 (s, 1H), 8.70 (d, J=0.9 Hz, 1H), 7.98-7.93 (m, 2H), 7.58-7.55 (m, 2H), 7.46-7.42 (m, 2H), 7.42-7.38 (m, 2H), 7.38-7.34 (m, 1H), 7.20 (d, J=2.7 Hz, 1H), 6.88 (d, J=46.1 Hz, 1H), 5.29 (t, J=5.7 Hz, 1H), 4.55 (d, J=5.7 Hz, 2H); ¹³C NMR (151 MHz, DMSO) δ 157.1 (d, J=28.1 Hz), 153.7, 150.6, 143.5, 140.0 (d, J=2.1 Hz), 138.0 (d, J=21.0 Hz), 129.0, 128.9 (d, J=2.2 Hz), 128.7 (2C), 127.0 (2C), 126.8 (d, J=6.0 Hz, 2C), 125.7 (2C), 115.5, 95.9 (d, J=5.4 Hz), 94.0 (d, J=172.5 Hz), 62.6; HRMS (ES+, m/z): found 334.1357, calcd for C₂₀H₁₇N₃OF [M+H]+ 334.1356.

### (4-(4-Phenoxy-7H-pyrrolo[2,3-d]pyrimidin-6-yl)phenyl)methanol (9)

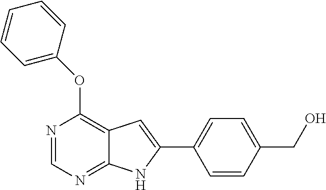 [SMILES: OCc1ccc(-c2cc3c(Oc4ccccc4)ncnc3[nH]2)cc1]

(4-(4-Phenoxy-7-((2-(trimethylsilyl)ethoxy)methyl)-7Hpyrrolo[2,3-d]pyrimidin-6-yl)phenyl)-methanol (80 mg, 1.8 mmol) was dissolved in DCM (10 mL), and then TFA (2 mL) was added slowly. The mixture was heated at 50° C. for 2 h, then cooled to 22° C., concentrated and co-evaporated with DCM/MeOH (1/1 v/v, 3×20 mL) to remove formaldehyde. Then, THF (11 mL) and sat. NaHCO₃ solution (11 mL) were added and the mixture was stirred for 90 min at 22° C. The mixture was diluted with water (20 mL) and EtOAc (30 mL), the phases were separated, and the water phase was extracted with more EtOAc (3×30 mL). The organic phase was washed with brine (80 mL) and dried over Na₂SO₄. The crude product was absorbed onto Celite 545 and purified using silica-gel flash chromatography (DCM/MeOH, 20:1, R_{f}=0.27). This gave 48 mg (0.15 mmol, 83%) of a colourless crystalline solid, mp. 268-272° C.; ¹H NMR (600 MHz, DMSO-d₆) δ: 12.67 (s, 1H), 8.30 (s, 1H), 7.90 (d, J=8.2 Hz, 2H), 7.49-7.46 (m, 2H), 7.41 (d, J=8.5 Hz, 2H), 7.30-7.27 (m, 3H), 6.97 (d, J=1.9 Hz, 1H), 5.26 (t, J=5.7 Hz, 1H), 4.54 (d, J=5.8 Hz, 2H); ¹³C NMR (150 Hz, DMSO-d₆) δ: 161.2, 154.7, 152.7, 150.3, 142.9, 137.7, 129.7 (2C), 129.3, 127.0 (2C), 125.3, 125.2 (2C), 122.0 (2C), 106.1, 94.6, 62.6; HRMS (ES+, m/z): found 318.1246 calcd for C₁₉H₁₆N₃O₂ [M+H]+ 318.1243.

### 4-Benzyl-6-(4-methoxyphenyl)-7H-pyrrolo[2,3-d]pyrimidine (10)

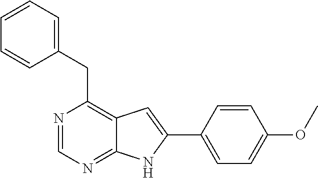 [SMILES: COc1ccc(-c2cc3c(Cc4ccccc4)ncnc3[nH]2)cc1]

The corresponding SEM protected intermediate (62 mg, 0.139 mmol), TFA (0.5 mL) and DCM (2.0 mL), oil-bath (50° C.), 1.5 h. Removed volatiles, added MeOH (6.0 mL) and aqueous ammonia (12.0 mL, 25%), rt, 1.5 h. Removed volatiles, purification by silica-gel column chromatography (DCM/MeOH—96:4, R_{f}=0.15) gave 42 mg (0.135 mmol, 97%) of a white solid. ¹H NMR (400 MHz, DMSO-d₆) δ 12.72 (s, 1H), 8.69 (s, 1H), 8.14-8.08 (m, 2H), 8.08-8.02 (m, 2H), 7.44-7.38 (m, 3H), 7.31-7.26 (m, 2H), 7.22-7.16 (m, 1H), 4.34 (s, 2H), 3.88 (s, 3H); ¹³C NMR (101 MHz, DMSO-d₆) δ 165.8, 160.8, 152.9, 151.8, 138.5, 137.1, 135.3, 129.8 (2C), 129.04 (2C), 128.4 (2C), 126.3, 125.6 (2C), 117.8, 98.4, 52.2, 41.1; HRMS (ASAP+, m/z): found 316.1454, calcd for C₂₀H₁₈N₃O [M+H]⁺ 316.1450.

### 6-Phenyl-N-(tetrahydro-2H-pyran-4-yl)-7H-pyrrolo[2,3-d]pyrimidin-4-amine (11)

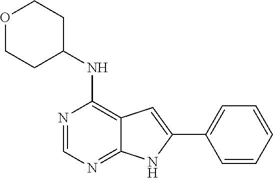 [SMILES: c1ccc(-c2cc3c(NC4CCOCC4)ncnc3[nH]2)cc1]

The reaction was run as described in general procedure C using the corresponding SEM-intermediate (89 mg, 0.208 mmol) for 3 h in the first step and 22 h with THF/water/NaHCO₃ in the basic step. The crude product was purified with three rounds of silica-gel column chromatography (CH₂Cl₂/MeOH, 9:1, R_{f}=0.35. This gave 27 mg (0.091 mmol, 44%) of a white powder, mp.>270° C. (decomp.); HPLC purity (method A): 97%; ¹H NMR (600 MHz, DMSO-d₆) δ 12.03 (s, 1H), 8.12 (s, 1H), 7.78 (d, J=6.9 Hz, 2H), 7.44 (t, J=7.8 Hz, 2H), 7.32 (d, J=7.7 Hz, 1H), 7.29 (t, J=7.4 Hz, 1H), 7.00 (d, J=2.2 Hz, 1H), 4.33-4.24 (m, 1H), 3.94-3.88 (m, 2H), 3.44 (td, J=11.7, 2.1 Hz, 2H), 1.94-1.88 (m, 2H), 1.57 (qd, J=12.2, 4.4 Hz, 2H); ¹³C NMR (150 MHz, DMSO-d₆) δ 155.0, 151.8, 151.4, 133.2, 131.7, 128.8 (2C), 127.1, 124.4 (2C), 103.8, 95.8, 66.2 (2C), 46.1, 32.9 (2C); IR (neat, cm⁻¹): 3132 (w), 2952 (w), 2839 (w), 1599 (s), 1471 (m), 1360 (m), 1141 (m), 904 (m), 754 (m); HRMS (ASAP+, m/z): found 295.1563, calcd for C₁₇H₁₉N₄O, [M+H]⁺, 295.1559.

### 6-(4-(Morpholinomethyl)phenyl)-N-(tetrahydro-2H-pyran-4-yl)-7H-pyrrolo[2,3-d]pyrimidin-4-amine (12)

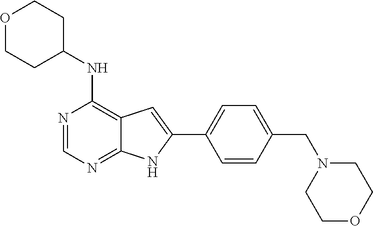 [SMILES: c1nc(NC2CCOCC2)c2cc(-c3ccc(CN4CCOCC4)cc3)[nH]c2n1]

The SEM-deprotection was performed as described in General procedure C starting with the corresponding SEM intermediate (103 mg, 0.197 mmol) and reacting for 4 h in the first step and 18 h in the second step. The crude product was purified by silica-gel column chromatography (CH₂Cl₂/MeOH, 9:1, R_{f}=0.26). This gave 69 mg, (0.176 mmol, 89%) as a white powder, mp.>277° C., purity >99%. ¹H NMR (600 MHz, DMSO-d₆) δ 11.99 (s, 1H), 8.11 (s, 1H), 7.73 (d, J=8.3 Hz, 2H), 7.36 (d, J=8.3 Hz, 2H), 7.31 (d, J=7.7 Hz, 1H), 6.98 (s, 1H), 4.35-4.21 (m, 1H), 3.94-3.88 (m, 2H), 3.60-3.56 (m, 4H), 3.47 (s, 2H), 3.44 (td, J=11.7, 2.1 Hz, 2H), 2.41-2.32 (m, 4H), 1.97-1.81 (m, 2H), 1.57 (qd, J=12.1, 4.4 Hz, 2H); ¹³C NMR (150 MHz, DMSO-d₆) δ 155.0, 151.8, 151.5, 136.9, 133.3, 130.6, 129.5 (2C), 124.4 (2C), 100.3, 95.7, 66.2 (4C), 62.1, 53.2 (2C), 46.1, 32.9 (2C); HRMS (ASAP+, m/z): found 394.2248, calcd for C₂₂H₂₈N₅O₂, [M+H]⁺, 394.2243.

### N-(4-(4-((2,3-Dihydro-1H-inden-1-yl)(methyl)amino)-7H-pyrrolo[2,3-d]pyrimidin-6-yl)phenyl)methanesulfonamide (13)

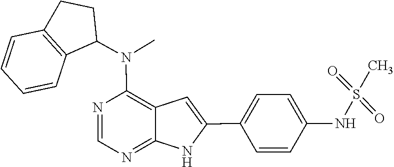 [SMILES: CN(c1ncnc2[nH]c(-c3ccc(NS(C)(=O)=O)cc3)cc12)C1CCc2ccccc21]

### N-(4-(4-Benzyl-7H-pyrrolo[2,3-d]pyrimidin-6-yl)phenyl)methanesulfonamide (14)

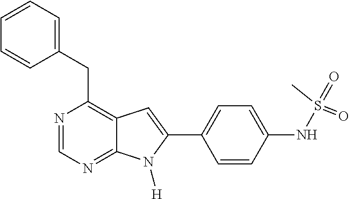 [SMILES: CS(=O)(=O)Nc1ccc(-c2cc3c(Cc4ccccc4)ncnc3[nH]2)cc1]

The compound was prepared as described in general procedure C, starting with the corresponding SEM-intermediate (75 mg, 0.147 mmol). The reaction times were 1.5 h and 16 h. Purification by silica-gel chromatography (MeOH/CH₂Cl₂, 8:92, R_{f}=0.26) gave 34.5 mg (0.091 mmol, 62%) as a white solid, mp>274.6° C. (decomp.). ¹H NMR (600 MHz, DMSO) δ 12.50 (s, 1H), 9.97 (s, 1H), 8.63 (s, 1H), 7.90 (d, J=8.6 Hz, 2H), 7.39 (d, J=7.8 Hz, 2H), 7.31-7.25 (m, 4H), 7.19 (t, J=7.3 Hz, 1H), 7.11 (s, 1H), 4.31 (s, 2H), 3.05 (s, 3H); ¹³C NMR (151 MHz, DMSO) δ 159.6, 152.7, 151.0, 138.8, 138.6, 138.1, 129.0 (2C), 128.4 (2C), 126.6 (2C), 126.3, 126.2, 119.6 (2C), 118.0, 95.4, 41.1, 39.1; IR (neat, cm⁻¹): 3237, 3113, 2852, 1579, 1500, 1329, 1299, 1150, 977, 761, 699, 503; HRMS (ES+, m/z): 379.1235 (calcd. C₂₀H₁₉N₄O₂S, 379.1229 [M+H]⁺)

### 4-Benzyl-6-phenyl-7H-pyrrolo[2,3-d]pyrimidine (15)

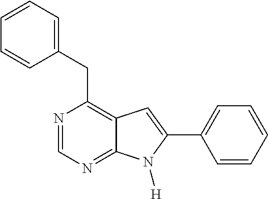 [SMILES: c1ccc(Cc2ncnc3[nH]c(-c4ccccc4)cc23)cc1]

The compound was prepared as described in general procedure C, starting with the corresponding SEM-intermediate (83 mg, 0.199 mmol). The reaction times were 1.5 h and 2 h. THF was removed in vacuo, and the precipitation was isolated by filtration, and thoroughly washed with water and n-pentane. After drying under reduced pressure this gave 45 mg (0.157 mmol, 79%) as a beige solid, mp>274.8° C. (decomp.). ¹H NMR (600 MHz, DMSO-d₆) δ 12.56 (s, 1H), 8.65 (s, 1H), 7.95 (d, J=7.7 Hz, 2H), 7.52-7.46 (m, 2H), 7.43-7.36 (m, 3H), 7.28 (td, J=7.6, 1.6 Hz, 2H), 7.22-7.16 (m, 2H), 4.33 (s, 2H); ¹³C NMR (151 MHz, DMSO-d₆) δ 159.9, 152.7, 151.1, 138.6, 138.4, 130.9, 129.1 (2C), 129.0 (2C), 128.5, 128.4 (2C), 126.3, 125.5 (2C), 117.9, 96.2, 41.1; IR (neat, cm⁻¹): 3108, 2986, 1580, 1491, 1346, 1260, 1178, 10777, 897, 755, 698, 562; HRMS (ES+, m/z): 286.1344 (calcd. C₁₉H₁₆N₃, 286.1344 [M+H]⁺)

### 4-(4-Benzyl-7H-pyrrolo[2,3-d]pyrimidin-6-yl)-N-methylbenzenesulfonamide (16)

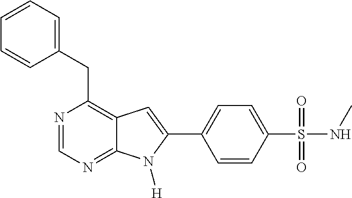 [SMILES: CNS(=O)(=O)c1ccc(-c2cc3c(Cc4ccccc4)ncnc3[nH]2)cc1]

The compound was prepared as described in general procedure C, starting with the corresponding SEM-intermediate (103 mg, 0.202 mmol). The reaction times were 2.5 h and 2.5 h. After filtration, the crude product was purified by silica-gel chromatography (gradient MeOH/CH₂Cl₂, 6:94 to 8:92, R_{f}=0.32) to obtain 16 mg (43.1 μmol, 46%) as a white solid, mp>263.6° C. (decomp.). ¹H NMR (600 MHz, DMSO-d₆) δ 12.74 (s, 1H), 8.70 (s, 1H), 8.16 (d, J=8.4 Hz, 2H), 7.86 (d, J=8.4 Hz, 2H), 7.51 (q, J=5.3 Hz, 1H), 7.44-7.39 (m, 3H), 7.31-7.24 (m, 2H), 7.22-7.17 (m, 1H), 4.34 (s, 2H), 2.45 (d, J=5.3 Hz, 3H); ¹³C NMR (151 MHz, DMSO-d₆) δ 160.8, 152.9, 151.8, 138.6, 138.5, 136.7, 134.6, 129.0 (2C), 128.4 (2C), 127.4 (2C), 126.4, 126.1 (2C), 117.8, 98.4, 41.1, 28.7; IR (neat, cm⁻¹): 3321, 3272, 2982, 1580, 1434, 1323, 1156, 1091, 819, 713, 561; HRMS (ES+, m/z): 379.1230 (calcd. C₂₀H₁₉N₄O₂S, 379.1229 [M+H]⁺)

### 4-Benzyl-6-(1-isopropyl-1H-pyrazol-4-yl)-7H-pyrrolo[2,3-d]pyrimidine (17)

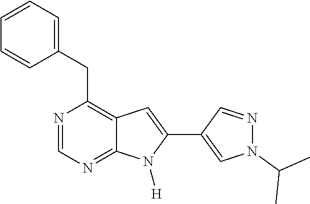 [SMILES: CC(C)n1cc(-c2cc3c(Cc4ccccc4)ncnc3[nH]2)cn1]

The compound was prepared as described in general procedure C, starting with the corresponding SEM-intermediate (69 mg, 0.155 mmol). The reaction times were 4.5 h and 2 h. THF was removed in vacuo, and the precipitation was isolated by filtration, and thoroughly washed with water and n-pentane. Drying under reduced pressure gave 30 mg (94.8 μmol, 61%) of an orange solid, mp. 213.3-218.2° C. (decomp.). ¹H NMR (600 MHz, DMSO-d₆) δ 12.28 (s, 1H), 8.57 (s, 1H), 8.31 (s, 1H), 7.99 (s, 1H), 7.37-7.34 (m, 2H), 7.29-7.26 (m, 2H), 7.20-7.17 (m, 1H), 6.79 (ap d, J=2.0 Hz, 1H), 4.53 (h, J=6.6 Hz, 1H), 4.26 (s, 2H), 1.45 (d, J=6.6 Hz, 6H); ¹³C NMR (151 MHz, DMSO-d₆) δ 158.3, 152.1, 150.3, 138.7, 136.3, 132.6, 128.9 (2C), 128.4 (2C), 126.3, 125.5, 118.0, 113.3, 93.4, 53.3, 41.1, 22.6 (2C); IR (neat, cm⁻¹): 3112, 2974, 1618, 1577, 1446, 1342, 1258, 982, 726, 563; HRMS (ES+, m/z): 318.1724 (calcd. C₁₉H₂₀N₅, 318.1719 [M+H]⁺)

### 4-Benzyl-6-(1,3-dimethyl-1H-pyrazol-4-yl)-7H-pyrrolo[2,3-d]pyrimidine (18)

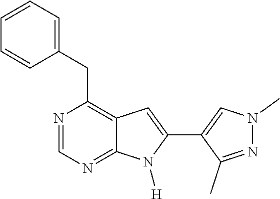 [SMILES: Cc1nn(C)cc1-c1cc2c(Cc3ccccc3)ncnc2[nH]1]

The compound was prepared as described in general section C, starting with the corresponding SEM-intermediate (81 mg, 0.187 mmol). The reaction times were 6 h and 17 h. Purification by silica-gel chromatography (gradient MeOH/CH₂Cl₂, 6:94 to 8:92, R_{f}=0.23) gave 48 mg (0.137 mmol, 73%) of a white solid, mp=195.9-198.3° C. (decomp.). ¹H NMR (600 MHz, DMSO-d₆) δ 12.14 (s, 1H), 8.58 (s, 1H), 8.06 (s, 1H), 7.40-7.36 (m, 2H), 7.31-7.25 (m, 2H), 7.22-7.16 (m, 1H), 6.54 (s, 1H), 4.31 (s, 2H), 3.81 (s, 3H), 2.35 (s, 3H); ¹³C NMR (151 MHz, DMSO-d₆) δ 158.6, 152.1, 150.3, 145.1, 138.8, 132.5, 129.7, 129.1 (2C), 128.3 (2C), 126.3, 118.0, 111.3, 94.4, 41.1, 38.5, 13.5; IR (neat, cm⁻¹): 3136, 3058, 2985, 1618, 1572, 1460, 1358, 1262, 1160, 897, 770, 699; HRMS (ES+, m/z): 304.1567 (calcd. C₁₈H₁₈N₅, 304.1562 [M+H]⁺)

### (4-(4-Benzyl-7H-pyrrolo[2,3-d]pyrimidin-6-yl)phenyl)dimethylphosphine oxide (19)

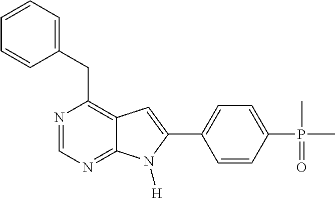 [SMILES: CP(C)(=O)c1ccc(-c2cc3c(Cc4ccccc4)ncnc3[nH]2)cc1]

The compound was prepared as described in general procedure C, starting the corresponding SEM-intermediate (52 mg, 0.105 mmol). The reaction times were 3 h and 2 h. THF was removed in vacuo, and the precipitation was isolated by filtration, and thoroughly washed with water and n-pentane. The product was dried under reduced pressure to obtain 23 mg (63.1 μmol, 60%) of a beige solid, mp>248.4° C. (decomp.). ¹H NMR (600 MHz, DMSO-d₆) δ 12.69 (s, 1H), 8.68 (s, 1H), 8.09 (dd, J=8.4, 2.1 Hz, 2H), 7.87 (dd, J=11.1, 8.2 Hz, 2H), 7.44-7.39 (m, 2H), 7.36 (s, 1H), 7.31-7.25 (m, 2H), 7.22-7.16 (m, 1H), 4.34 (s, 2H), 1.69 (d, J=13.3 Hz, 6H); ¹³C NMR (151 MHz, DMSO-d₆) δ 160.5, 152.8, 151.6, 138.5, 137.4, 135.9 (d, J=95.6 Hz), 133.4 (d, J=2.1 Hz), 130.5 (d, J=9.7 Hz, 2C), 129.1 (2C), 128.4 (2C), 126.3, 125.3 (d, J=11.4 Hz, 2C), 117.8, 97.6, 41.1, 17.7 (d, J=70.6 Hz, 2C); ³¹P NMR (243 MHz, DMSO-d₆) δ 33.4; IR (neat, cm⁻¹): 3352, 3114, 2982, 1579, 1433, 1166, 1115, 933, 700, 562; HRMS (ES+, m/z): 362.1422 (calcd. C₂₁H₂₁N₃OP, 362.1422 [M+H]⁺)

### Embodiments

A1. compound of formula (I)

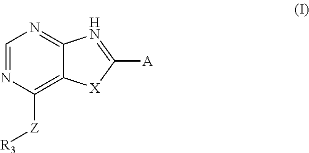

wherein:
   X is N or CH, preferably CH;
   Z is NR², 0, S, or CHR², wherein R² is hydrogen, halogen (preferably F), methyl or ethyl, or deuterated or partially deuterated methyl or ethyl;
   A is a 6-membered hydrocarbyl or heterocyclic ring which may be optionally substituted with at least one R¹ group;
   each R¹ is independently selected from halogen, hydroxyl, —OCF₃, —CF₃, —CF₂H, —OCF₂H, CH(CF₃)OH, —O—C₁₋₆-alkyl, —C₁₋₆alkyl-OH, —[C₁₋₆alkyl]ₘ-COOH, —[C₁₋₆alkyl]ₘ-COOC₁₋₆alkyl, —[C₁₋₆alkyl]ₘ-OCOC₁₋₆alkyl, —OCOR, —CO—[C₁₋₆alkyl]-COOH, —CO—[C₁₋₆alkyl]-COOR, C(CH₃)₂—CH₂OH, C(CH₃)₂OH, —NH₂, NHR, —NR₂, —NO₂, —SO₂NH₂, pyridyl (preferably ortho-pyridyl), cyclopropyl or cyclobutyl substituted with an —OH group, C₁-C₁₀ aliphatic hydrocarbyl group comprising at least one heteroatom selected from 0, or N, a —O—C₁-C₁₀ aliphatic hydrocarbyl group containing at least one heteroatom selected from O or N, or a —[CH₂]ₘ-heterocycle wherein said heterocycle is optionally substituted with ═O;
   each m is 0 or 1;
   R³ is a 5- or 6-membered hydrocarbyl or heterocyclic ring either of which may be optionally substituted with one or more groups independently selected from C₁-C₄ alkyl, hydroxyl, halogen, —CF₃, —CF₂H, —NR₂, —NHCOR, —CO₂H, —CO₂R, or —OR;
   each R is C₁-C₆ alkyl;
or a pharmaceutically acceptable salt or solvate thereof;
   with the provisos that
   when Z is O or NH, then A is not phenyl substituted with a meta-NH₂ group;
      and when Z is NH and A is phenyl, then R³ is not phenyl substituted with both —OH and —OMe.

A2. A compound as described in embodiment A1, wherein X is CH.

A3. A compound as described in any preceding embodiment, wherein Z is O or CHR², preferably CHR².

A4. A compound as described in any preceding embodiment, wherein R³ is phenyl optionally substituted with one or more groups independently selected from C₁-C₄ alkyl, hydroxyl, halogen, —CF₃, —CF₂H, —NR₂, —NHCOR, —CO₂H, —CO₂R, or —OR, preferably wherein R³ is phenyl optionally substituted with C₁-C₄ alkyl, halogen (preferably F) or —CF₃.

A5. A compound as described in any preceding embodiment, wherein R² is hydrogen, F or methyl, or deuterated or partially deuterated methyl.

A6. A compound as described in embodiment A1 of formula (II):

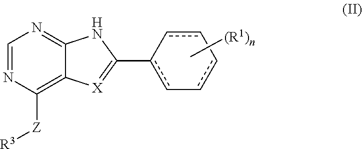

wherein:
   X, Z, R¹, R³, are as hereinbefore defined;
   each [symbol] independently represents a single or double bond, preferably
   each [symbol] represents a double bond;
   n is an integer between 0 and 3;
or a pharmaceutically acceptable salt or solvate thereof.

A7. A compound as described in any preceding embodiment, wherein R¹ is not —NH₂.

A8. A compound as described in any preceding embodiment, wherein each R¹ is selected from —O—C₁₋₆-alkyl and —C₁₋₆alkyl-OH.

A9. A compound as described in any preceding embodiment wherein n is an integer between 1 and 3, preferably n is 1

A10. A compound as described in any preceding embodiment wherein A is a 6-membered hydrocarbyl or heterocyclic ring which is substituted with at least one R¹ group.

A11. The compound of any preceding embodiment wherein A is substituted with 1 or 2 R¹ groups, preferably one R¹ group.

A12. A compound of formula (I)

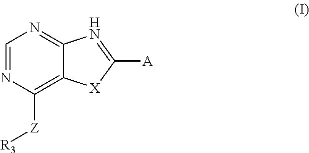

wherein:
   X is N or CH;
   Z is NR², O, S, or CHR², wherein R² is hydrogen, halogen (preferably F), methyl or ethyl, or deuterated or partially deuterated methyl or ethyl;
   A is a 6-membered hydrocarbyl or heterocyclic ring which may be optionally substituted with at least one R¹ group;
   each R¹ is independently selected from halogen, hydroxyl, —OCF₃, —CF₃, —CF₂H, —OCF₂H, CH(CF₃)OH, —O—C₁₋₆-alkyl, —C₁₋₆alkyl-OH, —[C₁₋₆alkyl]ₘ—COOH, —[C₁₋₆alkyl]ₘ-COOC₁₋₆alkyl, —[C₁₋₆alkyl]ₘ-OCOC₁₋₆alkyl, —OCOR, —CO—[C₁₋₆alkyl]-COOH, —CO—[C₁₋₆alkyl]-COOR, C(CH₃)₂—CH₂OH, C(CH₃)₂OH, —NH₂, NHR, —NR₂, —NO₂, —SO₂NH₂, pyridyl (preferably ortho-pyridyl), cyclopropyl or cyclobutyl substituted with an —OH group, C₁-C₁₀ aliphatic hydrocarbyl group comprising at least one heteroatom selected from O, or N, a —O—C₁-C₁₀ aliphatic hydrocarbyl group containing at least one heteroatom selected from O or N, or a —[CH₂]ₘ-heterocycle wherein said heterocycle is optionally substituted with ═O;
   each m is 0 or 1;
   R³ is a 5- or 6-membered hydrocarbyl or heterocyclic ring either of which may be optionally substituted with one or more groups independently selected from C₁-C₄ alkyl, hydroxyl, halogen, —CF₃, —CF₂H, —NR₂, —NHCOR, —CO₂H, —CO₂R, or —OR;
   each R is C₁-C₆ alkyl;
or a pharmaceutically acceptable salt or solvate thereof;
   with the proviso that
a) when Z is NH, and A is phenyl substituted with a meta-NH₂ group, then R³ is not phenyl substituted with one of the following:
   meta-OH and para-OMe
   meta-OH and para-Me
   3,4,5, tri-methoxy (i.e. —OMe in both meta-positions and the para-position)
   pyridyl
   meta-(NH)(COMe)
   meta-COOH
   meta-OH
   para-OH
   para-Me, and
b) when Z is O, and A is phenyl substituted with a meta-NH₂ group, then R³ is not meta-OH.

A13. A compound as described in embodiment A12, wherein X, Z, R¹, R³, are as defined in any of embodiments 1-11.

A14. A pharmaceutical composition comprising a compound as described in any preceding embodiment and at least one excipient.

A15. A pharmaceutical composition as described in embodiment A14 comprising at least one other chemotherapy agent.

A16. A compound as described in any of embodiments A1 to A13 for use in the treatment or prevention of a bone disorder, neurological disease, inflammatory disorder, cancer, or eye disease.

A17. The compound for use as described in embodiment A16, wherein
said bone disorder is osteoporosis, osteopetrosis, or osteosarcoma,
said neurological disease is Charcot-Marie-Tooth disease, Alzheimer's disease, amyotrophic lateral sclerosis, traumatic brain injury, or Hereditary diffuse leukoencephalopathy with spheroids,
said inflammatory disorder is rheumatoid arthritis or osteoarthritis;
said cancer is multiple myeloma, ovarian cancer, glioblastoma, breast cancer, malignant peripheral nerve sheath tumor; and/or
said eye disease is macular degeneration.

A18. The compound for use as described in embodiment A16 or A17, wherein said compound is for use in the treatment of cancer and is administered in combination with other anti-cancer agents or in combination with radiotherapy.

A19. A compound as described in any of embodiments A1 to A13 for use as a medicament.

A20. A method of treating or preventing a bone disorder, neurological disease, inflammatory disorder, cancer, or eye disease, comprising administering a compound as described in any of embodiments A1 to A13 to a subject in need thereof, optionally in combination with other chemotherapy agents or radiotherapy when administered for treating or preventing cancer.

A21. The use of a compound as described in any of embodiments A1 to A13 in the manufacture of a medicament for the treatment or prevention of a bone disorder, neurological disease, inflammatory disorder, cancer or eye disease.

A22. A process for the preparation of a compound as described in any of embodiments A1 to A13, wherein said process comprises the steps of:
reacting a compound of formula:

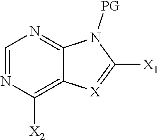

with
i) a compound of formula (RO)₂B-A or [A-BF₃]⁻ in the presence of a transition metal catalyst, wherein any OH groups in the R¹ substituent(s) of group A are optionally silane protected (e.g. with TBDMS), and
ii) a compound of formula —ZnBr—(CHR²)—R³ or —ZnBr—(CH₂)—R³ in the presence of a transition metal catalyst, or a compound of formula —OH—R³, —SH—R³, —(NHR²)—R³, or —(NH₂)—R³;
   in either order (i.e. i) then ii) or ii) then i));
then
iii) removing the protecting group PG;
   wherein
X₁ and X₂ are halogen;
PG is a protecting group, preferably selected from —SEM, THP, BOC, Cbz, Fmoc, SO₂Ph, Ts, MOM, CO₂H;
R is OH, OMe, or (RO)₂ is pinacol (i.e. —O—C(CH₃)₂—C(CH₃)₂—O—) or MIDA ester (i.e. —O—CO—CH₂—N(CH₃)—CH₂—CO—O—),
A, X, and R¹-R³ are as defined in any of embodiments A1 to A13.

A23. A process as described in embodiment A22, wherein if ZnBr—(CH₂)—R³ is used in step (ii), said process comprises an additional step of:
(ii′), reacting the resulting compound with a base followed by an alkylating agent (e.g. Mel or Etl) or fluorination agent (e.g. N-fluorobenzene-sulfonimide), wherein step (ii′) is after steps (i) and (ii).

1. A compound of formula (I) 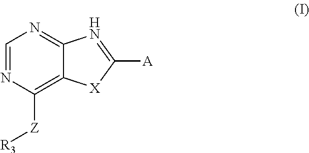
wherein:
X is CH or N;
Z is NR², O, S, or CHR², wherein R² is hydrogen, halogen, methyl or ethyl, or deuterated or partially deuterated methyl or ethyl;
A is a 5- or 6-membered hydrocarbyl or heterocyclic ring which may be optionally substituted with at least one R¹ group;
each R¹ is independently selected from halogen, hydroxyl, —OCF₃, —CF₃, —CF₂H, —OCF₂H, CH(CF₃)OH, —C₁₋₆-alkyl, —O—C₁₋₆-alkyl, —O—(C₁-C₆ alkyl)-CH₂F, —O—(C₁-C₆ alkyl)-CHF₂, —O—(C₁-C₆ alkyl)-CF₃, —C₁₋₆alkyl-OH, —[C₁₋₆alkyl]ₘ-COOH, —[C₁₋₆alkyl]ₘ-COOC₁₋₆alkyl, —[C₁₋₆alkyl]ₘ-OCOC₁₋₆alkyl, —OCOR, —CO—[C₁₋₆alkyl]-COOH, —CO—[C₁₋₆alkyl]-COOR, C(CH₃)₂—CH₂OH, —C(OH)₂(C₁₋₆alkyl-OH), —C(OH)(C₁₋₆alkyl-OH)₂, —C(C₁₋₆alkyl-OH)₃, C(CH₃)₂OH, —NH₂, NHR, —NR₂, —NO₂, —SO₂NH₂, —NHSO₂R, —NRSO₂R, —SO₂NHR, —SO₂NR₂, —P(O)R₂, pyridyl, cyclopropyl or cyclobutyl substituted with an —OH group, C₁-C₁₀ aliphatic hydrocarbyl group comprising at least one heteroatom selected from O, or N, a —O—C₁-C₁₀ aliphatic hydrocarbyl group containing at least one heteroatom selected from O or N, or a —[CH₂]ₘ-heterocycle wherein said heterocycle is optionally substituted with ═O or —OH;
each m is 0 or 1;
R³ is a 5- or 6-membered hydrocarbyl or heterocyclic ring either of which may be optionally substituted with one or more groups independently selected from C₁-C₄ alkyl, hydroxyl, halogen, —CF₃, —CF₂H, —NR₂, —NHCOR, —CO₂H, —CO₂R, or —OR; and
each R is C₁-C₆ alkyl;
or a pharmaceutically acceptable salt or solvate thereof;
with the provisos that
when Z is O or NH, then A is not phenyl substituted with a meta-NH₂ group;
and when Z is NH and A is phenyl, then R³ is not phenyl substituted with both —OH and —OMe.

2. The compound as claimed in claim 1, wherein X is CH.

3. compound as claimed in claim 1, wherein Z is O or CHR².

4. The compound as claimed in claim 1, wherein R³ is phenyl optionally substituted with one or more groups independently selected from C₁-C₄ alkyl, hydroxyl, halogen, —CF₃, —CF₂H, —NR₂, —NHCOR, —CO₂H, —CO₂R, or —OR.

5. The compound as claimed in claim 1, wherein R² is hydrogen, F or methyl, or deuterated or partially deuterated methyl.

6. The compound as claimed in claim 1, wherein the compound is of formula (II): 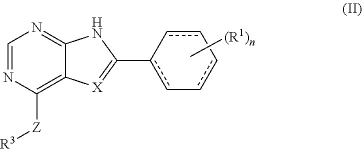
wherein:
each [symbol] independently represents a single or double bond; and
n is an integer between 0 and 3;
or a pharmaceutically acceptable salt or solvate thereof.

7. The compound as claimed in claim 6, wherein R¹ is not —NH₂.

8. The compound as claimed in claim 6, wherein each R¹ is selected from —O—C₁₋₆-alkyl and —C₁₋₆alkyl-OH.

9. The compound as claimed in claim 6, wherein n is an integer between 1 and 3.

10. The compound as claimed in claim 1, wherein A is a 6-membered hydrocarbyl or heterocyclic ring which is substituted with at least one R group.

11. (canceled)

12. The compound as claimed in claim 1, wherein:
a) when Z is NH, and A is phenyl substituted with a meta-NH₂ group, then R³ is not phenyl substituted with one of the following:
meta-OH and para-OMe
meta-OH and para-Me
3,4,5, tri-methoxy
pyridyl
meta-(NH)(COMe)
meta-COOH
meta-OH
para-OH
para-Me, and
b) when Z is O, and A is phenyl substituted with a meta-NH₂ group, then R³ is not meta-OH.

13. (canceled)

14. The compound as claimed in claim 1, wherein, when Z=NR₂, R³ is a 5- or 6-membered heterocyclic ring.

15. The compound as claimed in claim 1, wherein, when Z=NR₂, R³ is an O-based heterocyclic ring.

16. The compound as claimed in claim 1, wherein, when Z=O, R³ is unsubstituted phenyl and/or A is substituted with at least one R group.

17. (canceled)

18. The compound as claimed in claim 1, wherein R₃ is a 5- or 6-membered heterocyclic ring comprising one or two heteroatoms selected from O, N and/or S.

19. The compound as claimed in claim 1, wherein:
when Z is O, then A is not unsubstituted phenyl or unsubstituted pyridyl;
when Z is O, then A is not unsubstituted phenyl or unsubstituted pyridyl and R³ is not phenyl substituted with NH₂, NO₂ and/or F;
when X is N and Z is S, then R³ is not oxadiazole;
when X is N and R³ is a 5-membered heterocyclic ring, there are one or two heteroatoms in the 5-membered heterocyclic ring;
when X is N, Z is NH and A is unsubstituted phenyl, then R³ is not unsubstituted phenyl;
when X is N, Z is NH and R³ is unsubstituted phenyl, then A is not unsubstituted phenyl;
when X is N, R³ and A are not both unsubstituted phenyl;
when X is N and Z is O, R³ is not cyclopentyl;
when X is N, Z is O, A is not unsubstituted phenyl or unsubstituted pyridyl;
when X is N, Z is O, A is not unsubstituted phenyl or unsubstituted pyridyl;
when X is N and Z is O, R³ is not an unsubstituted 5-membered hydrocarbyl ring;
when X is N, Z is O or CHR², and in addition when Z is O, R³ is not an unsubstituted 5-membered hydrocarbyl ring;
when Z is S, then R³ is a 5- or 6-membered hydrocarbyl or heterocyclic ring either of which may be optionally substituted with one or more groups independently selected from C₁-C₄ alkyl, hydroxyl, halogen, —CF₃, —CF₂H, —NR₂, —CO₂H, —CO₂R, or —OR;
when X is N and Z is NH, R³ is a 5- or 6-membered hydrocarbyl ring or non-aromatic heterocyclic ring, any of which may be optionally substituted with one or more groups independently selected from C₁-C₄ alkyl, hydroxyl, halogen, —CF₃, —CF₂H, —NR₂, —NHCOR, —CO₂H, —CO₂R, or —OR;
when X is N and Z is NH, R¹ is not halogen;
when X is N and Z is NH, R¹ is independently selected from hydroxyl, —OCF₃, —CF₃, —CF₂H, —OCF₂H, CH(CF₃)OH, —C₁₋₆-alkyl, —O—C₁₋₆-alkyl, —O—(C₁-C₆ alkyl)-CH₂F, —O—(C₁-C₆ alkyl)-CHF₂, —O—(C₁-C₆ alkyl)-CF₃, —C₁₋₆alkyl-OH, —[C₁₋₆alkyl]ₘ—COOH, —[C₁₋₆alkyl]ₘ—COOC₁₋₆alkyl, —[C₁₋₆alkyl]ₘ-OCOC₁₋₆alkyl, —OCOR, —CO—[C₁₋₆alkyl]-COOH, —CO—[C₁₋₆alkyl]-COOR, C(CH₃)₂—CH₂OH, —C(OH)₂(C₁₋₆alkyl-OH), —C(OH)(C₁₋₆alkyl-OH)₂, —C(C₁₋₆alkyl-OH)₃C(CH₃)₂OH, —NH₂, NHR, —NR₂, —NO₂, —SO₂NH₂, —NHSO₂R, —NRSO₂R, —SO₂NHR, —SO₂NR₂, —POR₂, pyridyl, cyclopropyl or cyclobutyl substituted with an —OH group, C₁-C₁₀ aliphatic hydrocarbyl group comprising at least one heteroatom selected from O, or N, a —O—C₁-C₁₀ aliphatic hydrocarbyl group containing at least one heteroatom selected from O or N, or a —[CH₂]ₘ-heterocycle wherein said heterocycle is optionally substituted with ═O or —OH;
when X is N and Z is NH, R³ is not a 5- or 6-membered aromatic heterocyclic ring;
when X is N and Z is NH, A is not unsubstituted pyridyl;
when Z is NR², R³ is not cyclohexyl;
when X is CH and Z is NCH₃, R³ is not unsubstituted cyclohexyl;
when X is CH and Z is NCH₃, A is not unsubstituted phenyl;
X is CH and Z is NH, R¹ is not —OMe, hydroxyl or bromine;
when X is CH, Z is NR², O, S, or CHR², and R² is hydrogen, halogen, methyl or ethyl, or deuterated or partially deuterated methyl or ethyl, then NR₂ is not NH;
when X is CH and Z is O, R³ is not phenyl optionally substituted with one or more groups selected from OH and —OMe;
when X is CH and Z is O or S, R³ is a 5-membered hydrocarbyl or heterocyclic ring either of which may be optionally substituted with one or more groups independently selected from C₁-C₄ alkyl, hydroxyl, halogen, —CF₃, —CF₂H, —NR₂, —NHCOR, —CO₂H, —CO₂R, or —OR; or R³ is a 6-membered hydrocarbyl or heterocyclic ring either of which is substituted by one or more groups independently selected from C₁-C₄ alkyl, halogen, —CF₃, —CF₂H, —NR₂, —NHCOR, —CO₂H, —CO₂R or —CO₂H;
when X is CH and Z is NCH₃, A is a 5-membered hydrocarbyl or heterocyclic ring or 6-membered heterocyclic ring, each of which may be optionally substituted with at least one R¹ group or A is a 6-membered hydrocarbyl ring substituted with at least one R¹ group;
when X is CH, Z is O or CHR²;
when X is CH, Z is not NH and/or NMe;
when X is CH, R¹ is not NH₂ and there is at least one R¹ group;
when X is CH and Z is NCH₃, R³ is not cyclohexyl;
when X is CH and Z is NCH₃, A is not unsubstituted phenyl;
when A is phenyl there is at least one R¹ substituent;
when X is CH and Z is NH, R³ is not phenyl optionally substituted with one or more groups independently selected from C₁-C₄ alkyl, hydroxyl, halogen, —CF₃, —CF₂H, —NR₂, —NHCOR, —CO₂H, —CO₂R or —OR; and R³ is not substituted pyrazole;
when X is CH and Z is NH, R³ is not phenyl, pyrazole, pyridyl or pyridone optionally substituted with one or more groups independently selected from C₁-C₄ alkyl, hydroxyl, halogen, —CF₃, —CF₂H, —NR₂, —NHCOR, —CO₂H, —CO₂R, or —OR;
when X is CH and Z is NH, R¹ is not —CH₂OH, —CH₂NH₂, —COOC₁₋₆alkyl, —CH₂N—(C₁₋₆alkyl)₂, —CH₂NH—(C₁-C₁₀ aliphatic hydrocarbon containing at least one N and/or O), —CH₂NHCO-pyridyl, —CON—(C₁₋₆alkyl)₂, —CO-(heterocycle containing at least one N), —CH₂-(heterocycle containing at least one N and/or O), wherein said heterocycle is optionally substituted with one or more R groups;
or a combination thereof.

20.-52. (canceled)

53. The compound as claimed in claim 1, wherein the compound is selected from any one of the following compounds:

| | |
|---|---|
| | 5a-k and 9 |
| 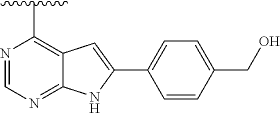 [SMILES: *c1ncnc2[nH]c(-c3ccc(CO)cc3)cc12] | |
| | 10 |
| 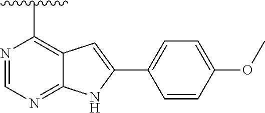 [SMILES: *c1ncnc2[nH]c(-c3ccc(OC)cc3)cc12] | |
| Compound | Compound | |
|---|---|---|
| 5a (TIA03-170) | SH-01-81 | 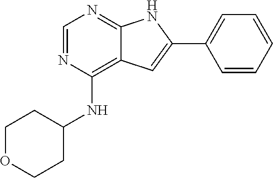 [SMILES: c1ccc(-c2cc3c(NC4CCOCC4)ncnc3[nH]2)cc1] |
| 5b (VT-44-03) | SH-01-98 | 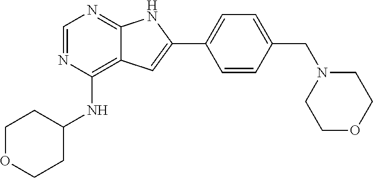 [SMILES: c1nc(NC2CCOCC2)c2cc(-c3ccc(CN4CCOCC4)cc3)[nH]c2n1] |
| 5c (VT-84-06) | LR-2-144 | 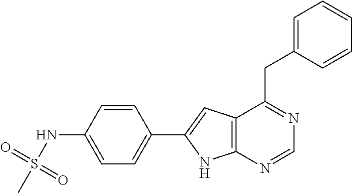 [SMILES: CS(=O)(=O)Nc1ccc(-c2cc3c(Cc4ccccc4)ncnc3[nH]2)cc1] |
| 5d (VT-22-03) | LR-2-163 | 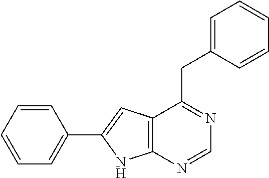 [SMILES: c1ccc(Cc2ncnc3[nH]c(-c4ccccc4)cc23)cc1] |
| 5e (VT-64-03) | LR-2-177 | 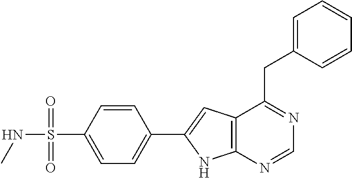 [SMILES: CNS(=O)(=O)c1ccc(-c2cc3c(Cc4ccccc4)ncnc3[nH]2)cc1] |
| 5f(VT-82-03) | LR-2-179 | 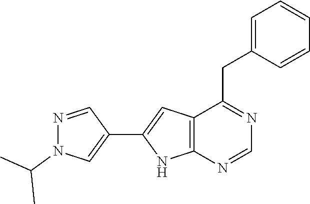 [SMILES: CC(C)n1cc(-c2cc3c(Cc4ccccc4)ncnc3[nH]2)cn1] |
| 5g (VT-28-02) | LR-2-185 | 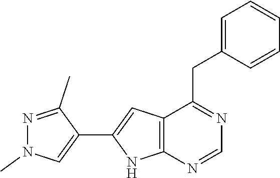 [SMILES: Cc1nn(C)cc1-c1cc2c(Cc3ccccc3)ncnc2[nH]1] |
| 5h (VT-56-03) | LR-3-002 | |
| | |
|---|---|
| 5i (VT-12-03; VT-10-05) | 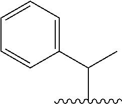 [SMILES: *C(C)c1ccccc1] |
| 5j (TIA05-134) | 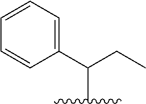 [SMILES: *C(CC)c1ccccc1] |
| 5k (TIA05-136) | 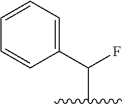 [SMILES: *C(F)c1ccccc1] |
| 9 (HIL120) | 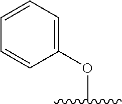 [SMILES: *Oc1ccccc1] |
| 10 (TIA03-164) | 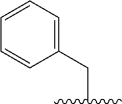 [SMILES: *Cc1ccccc1] |

54. A pharmaceutical composition comprising the compound as claimed in claim 1 and at least one excipient, and optionally further comprising at least one other chemotherapy agent.

55.-59. (canceled)

60. A method of treating or preventing a bone disorder, neurological disease, inflammatory disorder, cancer, or eye disease, comprising administering the compound as claimed in claim 1 to a subject in need thereof, optionally in combination with other chemotherapy agents or radiotherapy when administered for treating or preventing cancer.

61. (canceled)

62. A process for the preparation of the compound as claimed in claim 1, wherein said process comprises the steps of:
reacting a compound of formula: 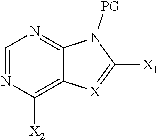
with
i) a compound of formula (RO)₂B-A or [A-BF₃]⁻ in the presence of a transition metal catalyst, wherein any OH groups in the R¹ substituent(s) of group A are optionally silane protected, and
ii) a compound of formula —ZnBr—(CHR²)—R³ or —ZnBr—(CH₂)—R³ in the presence of a transition metal catalyst, or a compound of formula —OH—R³, —SH—R³, —(NHR²)—R³, or —(NH₂)—R³;
in either order;
then
iii) removing the protecting group PG;
wherein
X₁ and X₂ are halogen;
PG is a protecting group; and
R is OH, OMe, or (RO)₂ is pinacol.

63. (canceled)
